(19) United States
(12) Patent Application Publication
(10) Pub. No.: US 2022/0135684 A1
(43) Pub. Date: May 5, 2022

(54) BISPECIFIC ANTIBODIES THAT BIND PD-L1 AND CD28

(71) Applicant: Xencor, Inc., Monrovia, CA (US)

(72) Inventors: John R. Desjarlais, Pasadena, CA (US); Gregory Moore, Azusa, CA (US); Michael Hedvat, Encino, CA (US); Juan Diaz, Anaheim Hills, CA (US); Veronica Gusti Zeng, Duarte, CA (US); Umesh S. Muchhal, Monrovia, CA (US)

(21) Appl. No.: 17/501,846

(22) Filed: Oct. 14, 2021

Related U.S. Application Data
(60) Provisional application No. 63/091,806, filed on Oct. 14, 2020.
(60) Provisional application No. 63/110,916, filed on Nov. 6, 2020.
(60) Provisional application No. 63/173,242, filed on Apr. 9, 2021.

(51) Int. Cl. C07K 16/28; A61K 39/00

(52) U.S. Cl. CPC C07K 16/2818; C07K 16/2827; C07K 2317/31; C07K 2317/622; A61K 2039/505; C07K 2317/92

(57) ABSTRACT

Provided herein are novel αPD-L1 antibodies and methods of using such antibodies for the treatment of cancers. In some embodiments, the antibodies are αPD-L1×αCD28 bispecific antibodies. Such antibodies enhance anti-tumor activity by providing a costimulatory signal for T-cell activation against tumor cells while advantageously also blocking inhibitory PD-L1:PD-1 pathway interactions.

### PRIORITY

This application claims the benefit of U.S. Provisional Patent Application Nos. 63/091,806, filed Oct. 14, 2020, 63/110,916, filed Nov. 6, 2020 and 63/173,242, filed Apr. 9, 2021, which are hereby incorporated by reference in their entireties.

### SEQUENCE LISTING INCORPORATION PARAGRAPH

The instant application contains a Sequence Listing which has been submitted electronically in ASCII format and is hereby incorporated by reference in its entirety. Said ASCII copy, created on Oct. 12, 2021, is named 067461-5275-WO_SL.txt and is 1,521,746 bytes in size.

### BACKGROUND

The natural immune response against tumor dispatches immune effector cells such as natural killer (NK) cells and T cells to attack and destroy tumor cells. Tumor infiltrating lymphocytes (TILs) often express multiple immune checkpoint receptors (e.g., PD-1, CTLA-4) and costimulatory receptors (e.g., ICOS, 4-1BB, OX40, GITR, and CD28). TILs lose their cytotoxic ability over time due to upregulation of inhibitory immune checkpoints. While checkpoint blockade has demonstrated increased clinical response rates relative to other treatment options, many patients still fail to achieve a response to checkpoint blockade. Engagement of costimulatory receptors on TILs could provide a positive signal capable of overcoming negative signals of immune checkpoints. Preclinical and clinical studies of agonistic costimulatory receptor antibodies have indeed demonstrated that agonism of costimulatory receptors can result in impressive anti-tumor responses, activating T cells to attack tumor cells.

It is also important for cancer therapy to enhance anti-tumor activity by specifically destroying tumor cells while minimizing peripheral toxicity. In this context, it is crucial that only T cells in the presence of the target tumor cells are provided a costimulatory signal. However, agonism of costimulatory receptors with monospecific full-length antibodies is likely nondiscriminatory with regards to TILs vs. peripheral T cells vs. autoantigen-reactive T cells that contribute to autoimmune toxicities. For instance, urelumab, a monospecific, nondiscriminatory, pan-4-1BB agonist antibody, exhibited significant liver toxicity in early phase clinical trials (Segal et al., 2016). Thus, there remains a need for novel immune response enhancing compositions for the treatment of cancers.

### SUMMARY

Provided herein are novel αPD-L1 antibodies. In some embodiments, the antibodies are αPD-L1×αCD28 bispecific antibodies. Such antibodies enhance anti-tumor activity by providing a costimulatory signal for T-cell activation against tumor cells while advantageously also blocking inhibitory PD-L1: PD1 pathway interactions (see FIG. 34). In some embodiments, such αPD-L1×αCD28 bispecific antibodies are useful for the treatment of cancers in conjunction with αCD3×αtumor target antigen (TTA) bispecific antibodies.

In a first aspect, provided herein is a heterodimeric antibody that comprises: a) a first monomer, b) a second monomer, and c) a light chain. The first monomer comprises: i) a scFv comprising a first variable heavy domain, an scFv linker and a first variable light domain; and ii) a first Fc domain, wherein the scFv is covalently attached to the N-terminus of the first Fc domain using a domain linker. The second monomer comprises, from N- to C-terminus, a VH1-CH1-hinge-CH2-CH3, wherein VH is a first variable heavy domain and CH2-CH3 is a second Fc domain. The light chain comprises, from N- to C-terminus, VL1-CL, wherein VL1 is a variable light domain and CL is a constant light domain. The VH1 and the VL1 form a first antigen binding domain (ABD). The scFv comprises a second VH domain (VH2), a scFv linker, and a second VL domain (VL2) and the VH2 and the VL2 form a second ABD. Further, one of the ABDs binds CD28 and the other binds PD-L1.

In some embodiments, the scFv comprises, from N- to C-terminus, VH2-scFv linker-VL2. In other embodiments, the scFv comprises, from N- to C-terminus, VL2-scFv linker-VH2.

In some embodiments, the first ABD binds PD-L1, wherein the amino acid sequence of the VH1 is selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and wherein the amino acid sequence of the VL1 is selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof.

In certain embodiments, the first ABD binds PD-L1, and the VH1 and VL1 are selected from the group consisting of: a VH1 having an amino acid sequence of SEQ ID NO:1183 and a VL1 having an amino acid sequence of SEQ ID NO:1187; a VH1 having an amino acid sequence of SEQ ID NO:1042 and a VL1 having an amino acid sequence of SEQ ID NO:1047; a VH1 having an amino acid sequence of SEQ ID NO:1159 and a VL1 having an amino acid sequence of SEQ ID NO:1163; a VH1 having an amino acid sequence of SEQ ID NO:1167 and a VL1 having an amino acid sequence of SEQ ID NO:1171; a VH1 having an amino acid sequence of SEQ ID NO:1175 and a VL1 having an amino acid sequence of SEQ ID NO:1179; a VH1 having an amino acid sequence of SEQ ID NO:1191 and a VL1 having an amino acid sequence of SEQ ID NO:1195; a VH1 having an amino acid sequence of SEQ ID NO:1199 and a VL1 having an amino acid sequence of SEQ ID NO:1203; a VH1 having an amino acid sequence of SEQ ID NO:1207 and a VL1 having an amino acid sequence of SEQ ID NO:1211; a VH1 having an amino acid sequence of SEQ ID NO:1215 and a VL1 having an amino acid sequence of SEQ ID NO:1219; a VH1 having an amino acid sequence of SEQ ID NO:1223 and a VL1 having an amino acid sequence of SEQ ID NO:1227; a VH1 having an amino acid sequence of SEQ ID NO:1231 and a VL1 having an amino acid sequence of SEQ ID NO:1235; a VH1 having an amino acid sequence of SEQ ID NO:1239 and a VL1 having an amino acid sequence of SEQ ID NO:1243; a VH1 having an amino acid sequence of SEQ ID NO:1247 and a VL1 having an amino acid sequence of SEQ ID NO:1251; a VH1 having an amino acid sequence of SEQ ID NO:1255 and a VL1 having an amino acid sequence of SEQ ID NO:1259; a VH1 having an amino acid sequence of SEQ ID NO:1263 and a VL1 having an amino acid sequence of SEQ ID NO:1267; a VH1 having an amino acid sequence of SEQ ID NO:1271 and a VL1 having an amino acid sequence of SEQ ID NO:1275; a VH1 having an amino acid sequence of SEQ ID NO:1279 and a VL1 having an amino acid sequence of SEQ ID NO:1283; a VH1 having an amino acid sequence of SEQ ID NO:1287 and a VL1 having an amino acid sequence of SEQ ID NO:1291; a VH1 having an amino acid sequence of SEQ ID NO:1295 and a VL1 having an amino acid sequence of SEQ ID NO:1299; and a VH1 having an amino acid sequence of SEQ ID NO:1303 and a VL1 having an amino acid sequence of SEQ ID NO:1307.

In exemplary embodiments, the second ABD binds to human CD28, wherein the amino acid sequence of the VH2 is selected from the group consisting of: SEQ ID NO:998, SEQ ID NO:984, SEQ ID NO:994 and SEQ ID NOs:561-629 or a variant thereof, and wherein the amino acid sequence of the VL2 is selected from the group consisting of: SEQ ID NO:1002, SEQ ID NO:984, and SEQ ID NOs:630-735 or a variant thereof.

In exemplary embodiments, the second ABD binds PD-L1, and the VH2 and VL2 are selected from the group consisting of: a VH2 having an amino acid sequence of SEQ ID NO:1030 and a VL2 having an amino acid sequence of SEQ ID NO:1034; a VH2 having an amino acid sequence of SEQ ID NO:1006 and a VL2 having an amino acid sequence of SEQ ID NO:1010; a VH2 having an amino acid sequence of SEQ ID NO:1014 and a VL2 having an amino acid sequence of SEQ ID NO:1018; and a VH2 having an amino acid sequence of SEQ ID NO:1022 and a VL2 having an amino acid sequence of SEQ ID NO:1026.

In some embodiments, the first Fc domain and second Fc domain are each variant Fc domains. In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In exemplary embodiments, the first and second Fc domains comprise heterodimerization skew variants S364K/E357Q: L368D/K370S.

In some embodiments, the first and second Fc domains each comprise one or more ablation variants. In some embodiments, the one or more ablation variants are E233P/L234V/L235A/G236del/S267K, wherein numbering is according to EU numbering.

In some embodiments, one of the first or second monomer further comprises one or more pI variants. In exemplary embodiments, the CH1-hinge-CH2-CH3 of the second monomer comprises pI variants N208D/Q295E/N384D/Q418E/N421D, wherein numbering is according to EU numbering.

In certain embodiments, the CH1-hinge-CH2-CH3 of the second monomer comprises amino acid variants L368D/K370S/N208D/Q295E/N384D/Q418E/N421D/E233P/L234V/L235A/G236del/S267K, the first Fc domain comprises amino acid variants S364K/E357Q/E233P/L234V/L235A/G236del/S267K, and the numbering is according to EU numbering.

In some embodiments, the first and second Fc domains each comprise amino acid variants 428L/434S.

In one aspect, provided herein is heterodimeric antibody that comprises: a) a first monomer, b) a second monomer, and c) a light chain. The first monomer comprises from N- to C-terminus, VH1-CH1-first domain linker-scFv-second domain linker-CH2-CH3, wherein VH1 is a first variable heavy domain, and CH2-CH3 is a first Fc domain. The second monomer comprises from N- to C-terminus, a VH1-CH1-hinge-CH2-CH3, wherein CH2-CH3 is a second Fc domain. The light chain comprises from N- to C-terminus, VL1-CL, wherein VL1 is a variable light domain and CL is a constant light domain. Each of the VH1 domains and the first VL1 domain together form a first antigen binding domain (ABD). The scFv comprises a second VH domain (VH2), a scFv linker, and a second VL domain (VL2), and the VH2 and the VL2 form a second ABD. Further, one of the first and second ABDs bind human CD28 and the other of the first and second ABDs binds PD-L1.

In exemplary embodiments, the first ABDs bind human CD28 and the second ABD binds PD-L1. In some embodiments, the first ABDs bind PD-L1 and the second ABD binds human CD28.

In some embodiments, the scFv comprises, from N- to C-terminus, VL2-scFv linker-VH2. In other embodiments, the scFv comprises, from N- to C-terminus, VH2-scFv linker-VL2.

In some embodiments, the first ABDs bind PD-L1, wherein the amino acid sequence of the VH1 is selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and wherein the amino acid sequence of the VL1 is selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof.

In some embodiments, the first ABDs bind PD-L1, and the VH1 and VL1 are selected from the group consisting of: a VH1 having an amino acid sequence of SEQ ID NO:1183 and a VL1 having an amino acid sequence of SEQ ID NO:1187; a VH1 having an amino acid sequence of SEQ ID NO:1042 and a VL1 having an amino acid sequence of SEQ ID NO:1047; a VH1 having an amino acid sequence of SEQ ID NO:1159 and a VL1 having an amino acid sequence of SEQ ID NO:1163; a VH1 having an amino acid sequence of SEQ ID NO:1167 and a VL1 having an amino acid sequence of SEQ ID NO:1171; a VH1 having an amino acid sequence of SEQ ID NO:1175 and a VL1 having an amino acid sequence of SEQ ID NO:1179; a VH1 having an amino acid sequence of SEQ ID NO:1191 and a VL1 having an amino acid sequence of SEQ ID NO:1195; a VH1 having an amino acid sequence of SEQ ID NO:1199 and a VL1 having an amino acid sequence of SEQ ID NO:1203; a VH1 having an amino acid sequence of SEQ ID NO:1207 and a VL1 having an amino acid sequence of SEQ ID NO:1211; a VH1 having an amino acid sequence of SEQ ID NO:1215 and a VL1 having an amino acid sequence of SEQ ID NO:1219; a VH1 having an amino acid sequence of SEQ ID NO:1223 and a VL1 having an amino acid sequence of SEQ ID NO:1227; a VH1 having an amino acid sequence of SEQ ID NO:1231 and a VL1 having an amino acid sequence of SEQ ID NO:1235; a VH1 having an amino acid sequence of SEQ ID NO:1239 and a VL1 having an amino acid sequence of SEQ ID NO:1243; a VH1 having an amino acid sequence of SEQ ID NO:1247 and a VL1 having an amino acid sequence of SEQ ID NO:1251; a VH1 having an amino acid sequence of SEQ ID NO:1255 and a VL1 having an amino acid sequence of SEQ ID NO:1259; a VH1 having an amino acid sequence of SEQ ID NO:1263 and a VL1 having an amino acid sequence of SEQ ID NO:1267; a VH1 having an amino acid sequence of SEQ ID NO:1271 and a VL1 having an amino acid sequence of SEQ ID NO:1275; a VH1 having an amino acid sequence of SEQ ID NO:1279 and a VL1 having an amino acid sequence of SEQ ID NO:1283; a VH1 having an amino acid sequence of SEQ ID NO:1287 and a VL1 having an amino acid sequence of SEQ ID NO:1291; a VH1 having an amino acid sequence of SEQ ID NO:1295 and a VL1 having an amino acid sequence of SEQ ID NO:1299; and a VH1 having an amino acid sequence of SEQ ID NO:1303 and a VL1 having an amino acid sequence of SEQ ID NO:1307.

In some embodiments, the second ABD binds to human CD28, wherein the amino acid sequence of the VH2 is selected from the group consisting of: SEQ ID NO:998, SEQ ID NO:984, SEQ ID NO:994 and SEQ ID NOs:561-629 or a variant thereof, and wherein the amino acid sequence of the VL2 is selected from the group consisting of: SEQ ID NO:1002, SEQ ID NO:984, and SEQ ID NOs:630-735 or a variant thereof.

In some embodiments, the second ABD binds PD-L1, and the VH2 and VL2 are selected from the group consisting of: a VH2 having an amino acid sequence of SEQ ID NO:1030 and a VL2 having an amino acid sequence of SEQ ID NO:1034; a VH2 having an amino acid sequence of SEQ ID NO:1006 and a VL2 having an amino acid sequence of SEQ ID NO:1010; a VH2 having an amino acid sequence of SEQ ID NO:1014 and a VL2 having an amino acid sequence of SEQ ID NO:1018; and a VH2 having an amino acid sequence of SEQ ID NO:1022 and a VL2 having an amino acid sequence of SEQ ID NO:1026.

In some embodiments, the first Fc domain and second Fc domain are each variant Fc domains. In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In exemplary embodiments, the first and second Fc domains comprise heterodimerization skew variants S364K/E357Q: L368D/K370S.

In some embodiments, the first and second Fc domains each comprise one or more ablation variants. In some embodiments, the one or more ablation variants are E233P/L234V/L235A/G236del/S267K, wherein numbering is according to EU numbering.

In some embodiments, one of the first or second monomer further comprises one or more pI variants. In exemplary embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267K, the first Fe domain comprises amino acid variants S364K/E357Q/E233P/L234V/L235A/G236del/S267K, and numbering is according to EU numbering.

In some embodiments, the first and second Fc domains each comprise amino acid variants 428L/434S.

In one aspect, provided herein is heterodimeric antibody that comprises: a) a first monomer, b) a second monomer, and c) a light chain. The first monomer comprises from N-terminus to C-terminus, VH1-CH1-hinge-CH2-CH3-domain linker-scFv, wherein VH1 is a first variable heavy domain, and CH2-CH3 is a first Fc domain. The second monomer comprises from N-terminus to C-terminus a VH1-CH1-hinge-CH2-CH3, wherein CH2-CH3 is a second Fc domain. The light chain comprises, from N-terminus to C-terminus, VL1-CL, wherein VL1 is a variable light domain and CL is a constant light domain. Each of the VH1 domain and the first VL1 domain together form a first antigen binding domain (ABD). The scFv comprises a second VH domain (VH2), a scFv linker, and a second VL domain (VL2), and the VH2 and the VL2 together form a second ABD. Further, one of the first and second ABDs bind human CD28 and the other of the first and second ABDs bind PD-L1.

In exemplary embodiments, the first ABDs bind human CD28 and the second ABD binds PD-L1. In some embodiments, the first ABDs bind PD-L1 and the second ABD binds human CD28.

In some embodiments, the scFv comprises, from N- to C-terminus, VL2-scFv linker-VH2. In exemplary embodiments, the scFv comprises, from N- to C-terminus, VH2-scFv linker-VL2.

In some embodiments, the first ABDs bind PD-L1, wherein the amino acid sequence of the VH1 is selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and wherein the amino acid sequence of the VL1 is selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof.

In some embodiments, the first ABDs bind PD-L1, and the VH1 and VL1 are selected from the group consisting of: a VH1 having an amino acid sequence of SEQ ID NO:1183 and a VL1 having an amino acid sequence of SEQ ID NO:1187; a VH1 having an amino acid sequence of SEQ ID NO:1042 and a VL1 having an amino acid sequence of SEQ ID NO:1047; a VH1 having an amino acid sequence of SEQ ID NO:1159 and a VL1 having an amino acid sequence of SEQ ID NO:1163; a VH1 having an amino acid sequence of SEQ ID NO:1167 and a VL1 having an amino acid sequence of SEQ ID NO:1171; a VH1 having an amino acid sequence of SEQ ID NO:1175 and a VL1 having an amino acid sequence of SEQ ID NO:1179; a VH1 having an amino acid sequence of SEQ ID NO:1191 and a VL1 having an amino acid sequence of SEQ ID NO:1195; a VH1 having an amino acid sequence of SEQ ID NO:1199 and a VL1 having an amino acid sequence of SEQ ID NO:1203; a VH1 having an amino acid sequence of SEQ ID NO:1207 and a VL1 having an amino acid sequence of SEQ ID NO:1211; a VH1 having an amino acid sequence of SEQ ID NO:1215 and a VL1 having an amino acid sequence of SEQ ID NO:1219; a VH1 having an amino acid sequence of SEQ ID NO:1223 and a VL1 having an amino acid sequence of SEQ ID NO:1227; a VH1 having an amino acid sequence of SEQ ID NO:1231 and a VL1 having an amino acid sequence of SEQ ID NO:1235; a VH1 having an amino acid sequence of SEQ ID NO:1239 and a VL1 having an amino acid sequence of SEQ ID NO:1243; a VH1 having an amino acid sequence of SEQ ID NO:1247 and a VL1 having an amino acid sequence of SEQ ID NO:1251; a VH1 having an amino acid sequence of SEQ ID NO:1255 and a VL1 having an amino acid sequence of SEQ ID NO:1259; a VH1 having an amino acid sequence of SEQ ID NO:1263 and a VL1 having an amino acid sequence of SEQ ID NO:1267; a VH1 having an amino acid sequence of SEQ ID NO:1271 and a VL1 having an amino acid sequence of SEQ ID NO:1275; a VH1 having an amino acid sequence of SEQ ID NO:1279 and a VL1 having an amino acid sequence of SEQ ID NO:1283; a VH1 having an amino acid sequence of SEQ ID NO:1287 and a VL1 having an amino acid sequence of SEQ ID NO:1291; a VH1 having an amino acid sequence of SEQ ID NO:1295 and a VL1 having an amino acid sequence of SEQ ID NO:1299; and a VH1 having an amino acid sequence of SEQ ID NO:1303 and a VL1 having an amino acid sequence of SEQ ID NO:1307.

In some embodiments, the second ABD binds to human CD28, wherein the amino acid sequence of the VH2 is selected from the group consisting of: SEQ ID NO:998, SEQ ID NO:984, SEQ ID NO:994 and SEQ ID NOs:561-629 or a variant thereof, and wherein the amino acid sequence of the VL2 is selected from the group consisting of: SEQ ID NO:1002, SEQ ID NO:984, and SEQ ID NOs:630-735 or a variant thereof.

In exemplary embodiments, the second ABD binds PD-L1, and the VH2 and VL2 are selected from the group consisting of: a VH1 having an amino acid sequence of SEQ ID NO:1030 and a VL2 having an amino acid sequence of SEQ ID NO:1034; a VH1 having an amino acid sequence of SEQ ID NO:1006 and a VL2 having an amino acid sequence of SEQ ID NO:1010; a VH2 having an amino acid sequence of SEQ ID NO:1014 and a VL2 having an amino acid sequence of SEQ ID NO:1018; and a VH2 having an amino acid sequence of SEQ ID NO:1022 and a VL2 having an amino acid sequence of SEQ ID NO:1026.

In some embodiments, the first Fc domain and second Fc domain are each variant Fc domains. In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In exemplary embodiments, the first and second Fc domains comprise heterodimerization skew variants S364K/E357Q: L368D/K370S.

In some embodiments, the first and second Fc domains each comprise one or more ablation variants. In some embodiments, the one or more ablation variants are E233P/L234V/L235A/G236del/S267K, wherein numbering is according to EU numbering.

In some embodiments, one of the first or second monomer further comprises one or more pI variants. In exemplary embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267K, the first Fe domain comprises amino acid variants S364K/E357Q/E233P/L234V/L235A/G236del/S267K, and numbering is according to EU numbering.

In some embodiments, the first and second Fc domains each comprise amino acid variants 428L/434S.

In one aspect, provided herein is composition comprising an anti-PD-L1 Antigen Binding Domain (ABD) comprising: a variable heavy domain (VH) with an amino acid sequence selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and variable light domain (VL) with an amino acid sequence selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof.

In another aspect, provided herein is a composition comprising an anti-PD-L1 Antigen Binding Domain (ABD) comprising a variable heavy domain (VH) and a variable light domain (VL) selected from the group consisting of: a VH having an amino acid sequence of SEQ ID NO:1183 and a VL having an amino acid sequence of SEQ ID NO:1187; a VH having an amino acid sequence of SEQ ID NO:1042 and a VL having an amino acid sequence of SEQ ID NO:1047; a VH having an amino acid sequence of SEQ ID NO:1159 and a VL having an amino acid sequence of SEQ ID NO:1163; a VH having an amino acid sequence of SEQ ID NO:1167 and a VL having an amino acid sequence of SEQ ID NO:1171; a VH having an amino acid sequence of SEQ ID NO:1175 and a VL having an amino acid sequence of SEQ ID NO:1179; a VH having an amino acid sequence of SEQ ID NO:1191 and a VL having an amino acid sequence of SEQ ID NO:1195; a VH having an amino acid sequence of SEQ ID NO:1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

In one aspect, provided herein is a composition comprising an anti-PD-L1 Antigen Binding Domain (ABD) comprising a variable heavy domain having an amino acid sequence of SEQ ID NO:1183 and a variable light domain having an amino acid sequence of SEQ ID NO: 1187.

In one aspect, provided herein is a composition comprising an anti-PD-L1 Antigen Binding Domain (ABD) comprising a variable heavy domain having an amino acid sequence of SEQ ID NO:1042 and a variable light domain having an amino acid sequence of SEQ ID NO: 1047.

In one aspect, provided herein is a composition comprising an anti-PD-L1 Antigen Binding Domain (ABD) comprising a variable heavy domain having an amino acid sequence of SEQ ID NO:1159 and a variable light domain having an amino acid sequence of SEQ ID NO: 1163.

In some embodiments, the composition is an antibody comprising: a) a heavy chain comprising, from N- to C-terminus: VH-CH1-hinge-CH2-CH3, wherein VH is the variable heavy domain and CH1-hinge-CH2-CH3 is heavy chain constant domain; and b) a light chain comprising from N- to C-terminus: VL-CL, wherein VL is the variable light domain and CL is a light chain constant domain.

Also provided herein are nucleic acid compositions encoding the compositions and antibodies provided herein, expression vectors that include such nucleic acids, and host cells that include the nucleic acids and expression vectors.

In another aspect, provided herein is a method of treating a cancer that includes administering to a patient in need thereof one of the subject antibodies provided herein. In some embodiments, the method further includes administering an anti-CD3×tumor target antigen bispecific antibody to the patient.

### BRIEF DESCRIPTION OF THE DRAWINGS

FIGS. 1A and 1B depict the sequences for human, mouse, and cynomolgus CD28. Such CD28 are useful for the development of cross-reactive CD28 antigen binding domains for ease of clinical development.

FIG. 2 depicts the sequences for human, mouse, and cynomolgus PD-L1. Such PD-L1 are useful for the development of cross-reactive CD28 antigen binding domains for ease of clinical development.

FIG. 3A-3F depict useful pairs of heterodimerization variant sets (including skew and pI variants). In FIG. 3F, there are variants for which there are no corresponding “monomer 2” variants. Such variants are pI variants that can be used alone on either monomer of a bispecific antibody (e.g., αPD-L1×αCD28 bsAb), or included, for example, on the non-scFv side of a format that utilizes an scFv as a component and an appropriate charged scFv linker can be used on the second monomer that utilizes an scFv as the CD28 binding domain. Suitable charged linkers are shown in FIG. 6.

FIG. 4 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 5 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 6 depicts a number of charged scFv linkers that find use in increasing or decreasing the pI of the subject heterodimeric bispecific antibodies that utilize one or more scFv as a component, as described herein (e.g., a PD-L1×αCD28 bsAbs). The (+H) positive linker finds particular use herein, particularly with anti-CD28 VL and VH sequences shown herein. A single prior art scFv linker with a single charge is referenced as “Whitlow,” from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIG. 7 depicts a number of exemplary domain linkers. In some embodiments, these linkers find use linking a single-chain Fv to an Fc chain. In some embodiments, these linkers may be combined in any orientation. For example, a GGGGS linker may be combined with a “lower half hinge” linker at the N-terminus or at the C-terminus.

FIG. 8 shows a particularly useful embodiment of the heterodimeric Fc domains (i.e. CH2-CH3 in this embodiment) of the anti-CD28×anti-PD-L1 bsAbs of the invention

FIG. 9 depicts various heterodimeric skew variant amino acid substitutions that can be used with the heterodimeric antibodies described herein.

FIGS. 10A-10C show the sequences of several useful heterodimeric αB7-H3×αCD28 bsAb backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 11 depicts illustrative sequences of heterodimeric B7H3×CD28 bsAb backbone for use in the 2+1 mAb-scFv format. The format depicted here is based on heterodimeric Fc backbone 1 as depicted in Figure X, except further including G446_on monomer 1 (−) and G446_/K447_on monomer 2 (+). It should be noted that any of the additional backbones depicted in Figure X may be adapted for use in the 2+1 mAb-scFv format with or without including K447_on one or both chains. It should be noted that these sequences may further include the M428L/N434S variants.

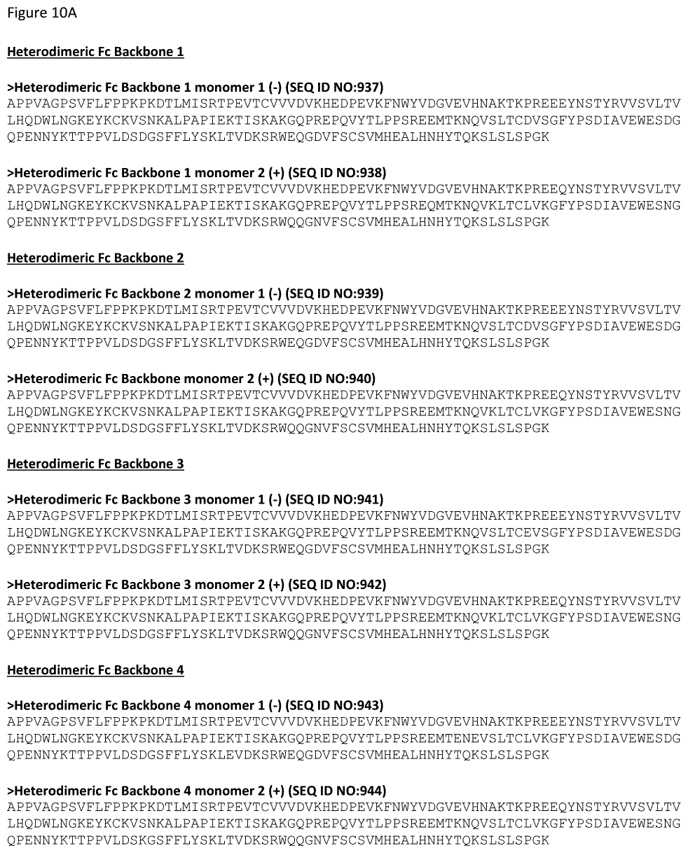

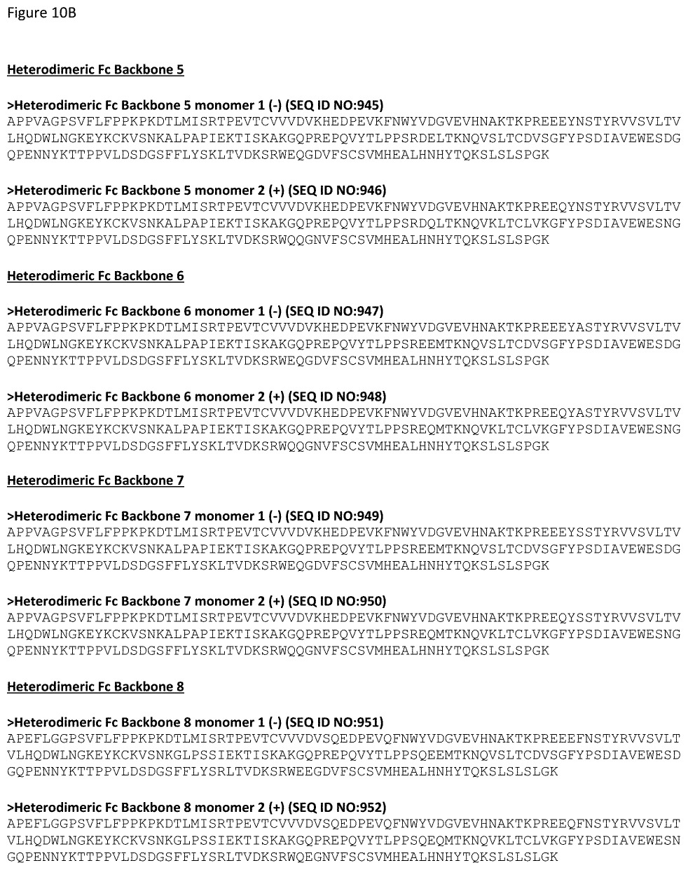

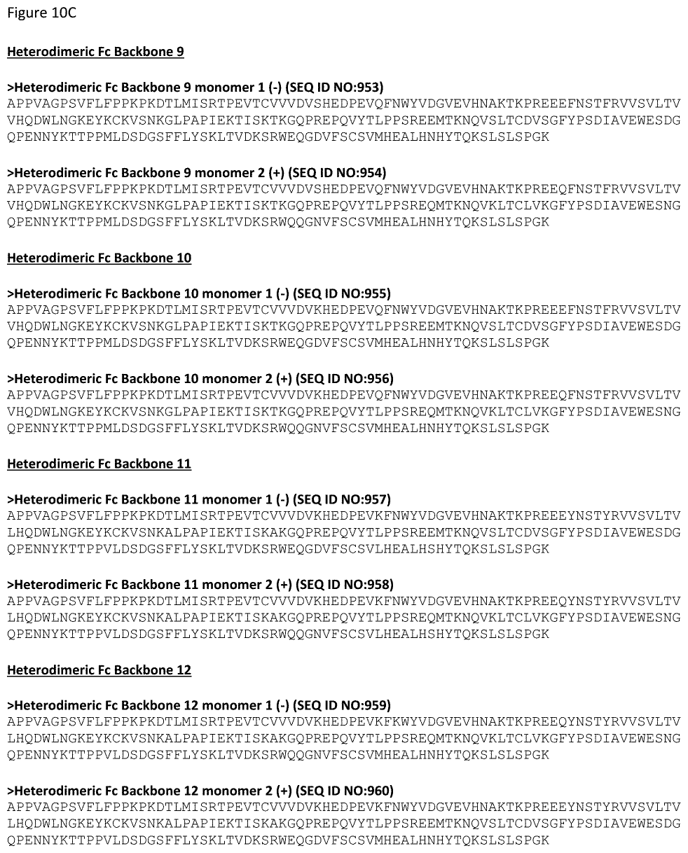

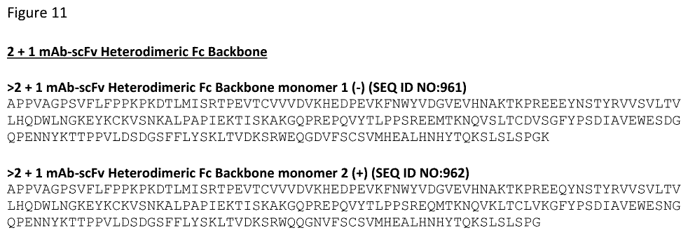

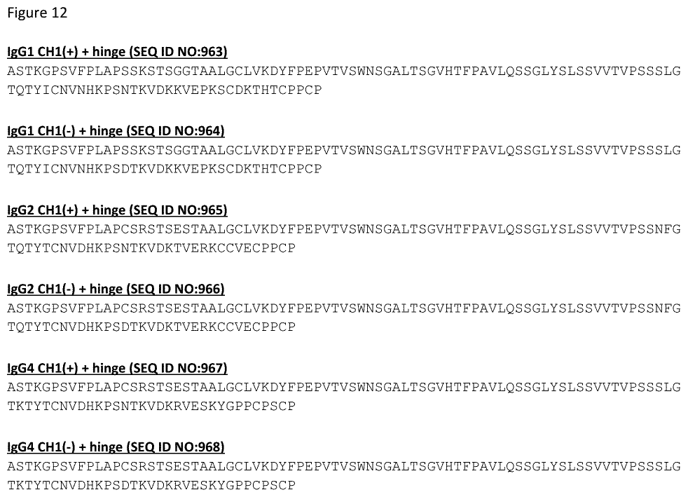

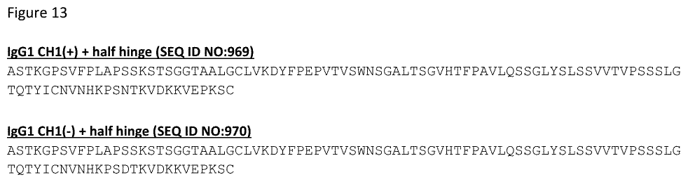

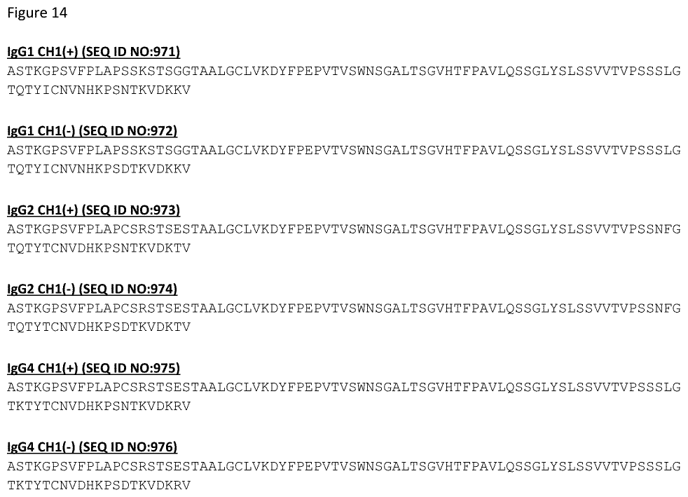

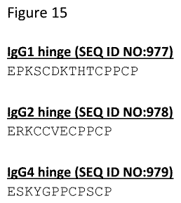

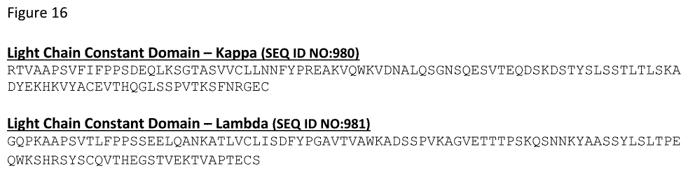

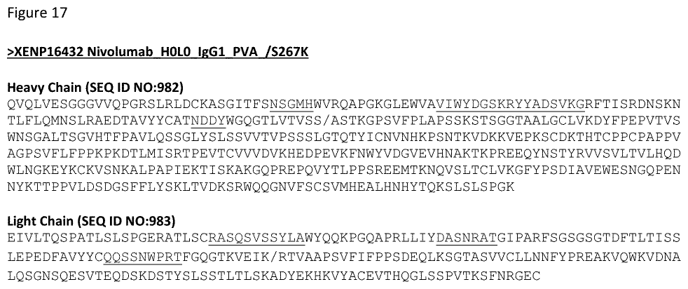

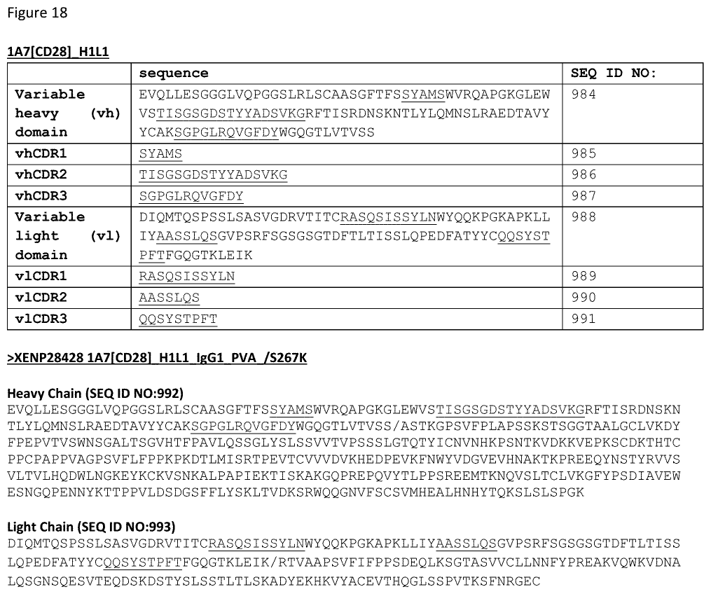

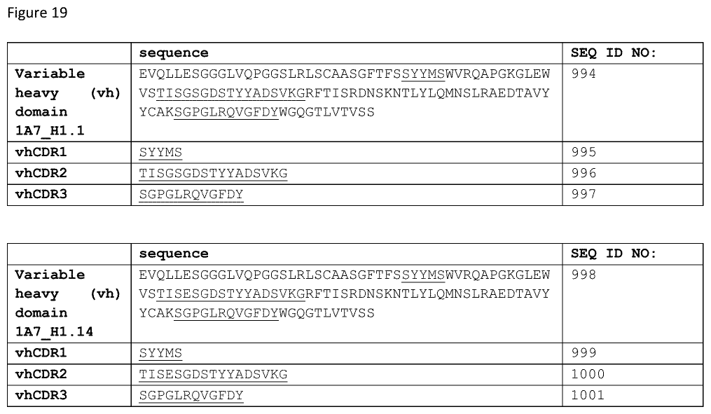

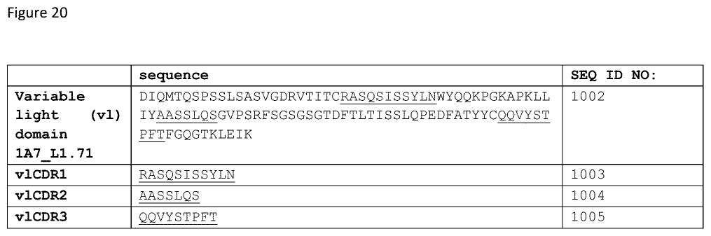

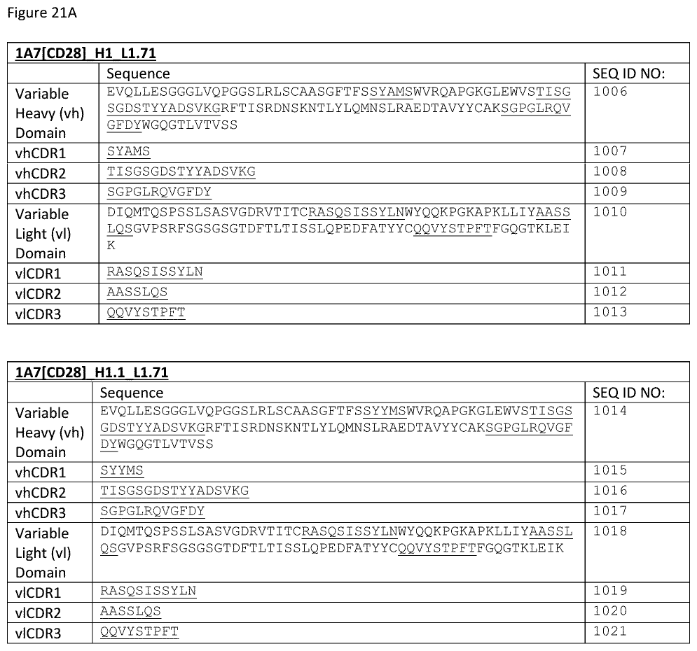

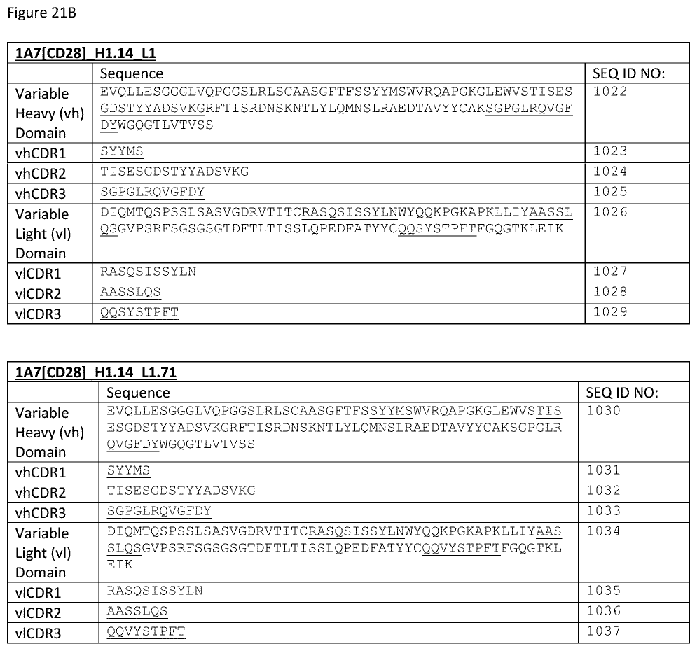

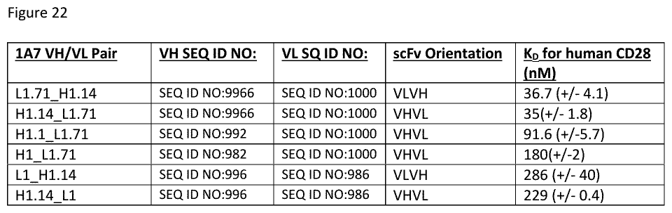

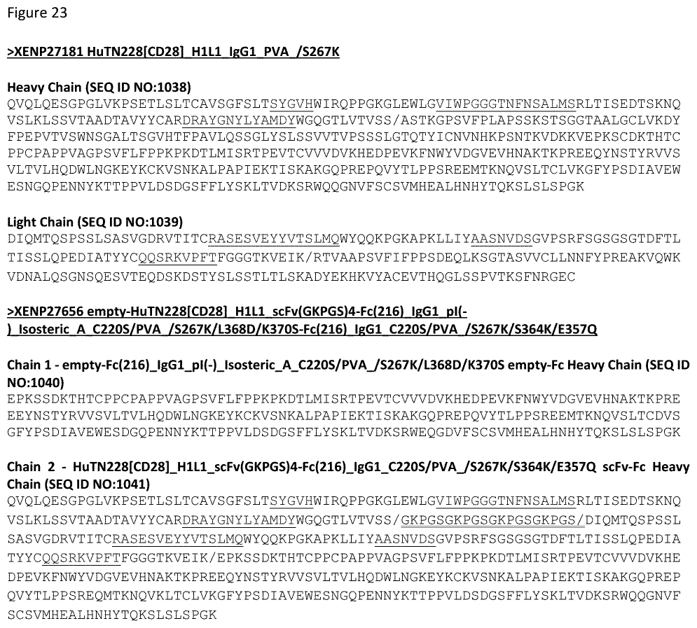

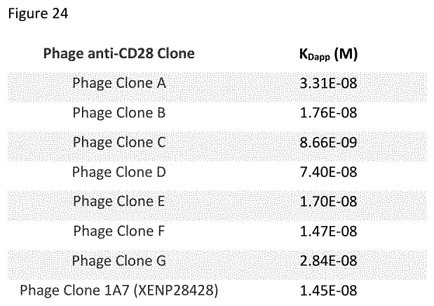

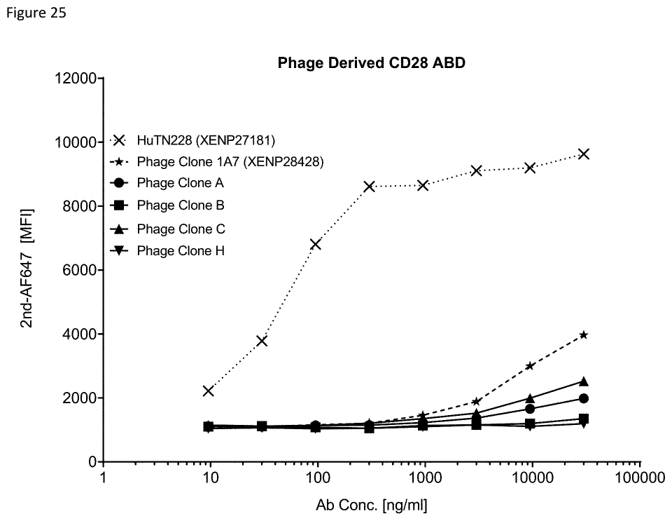

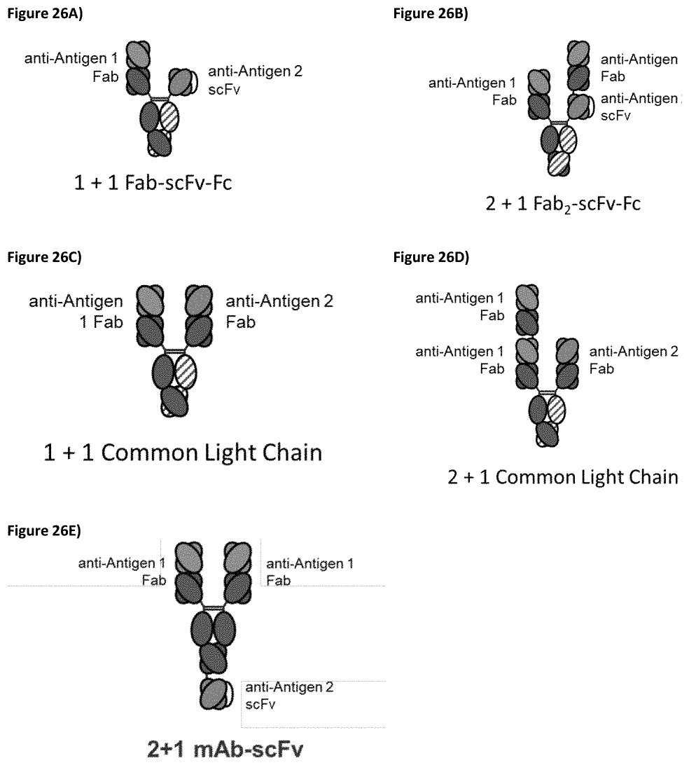

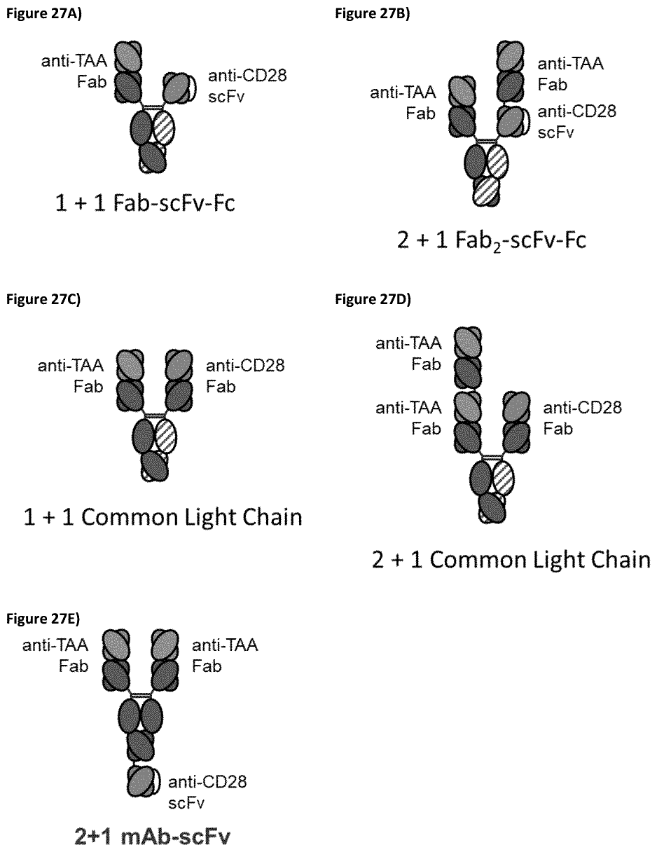

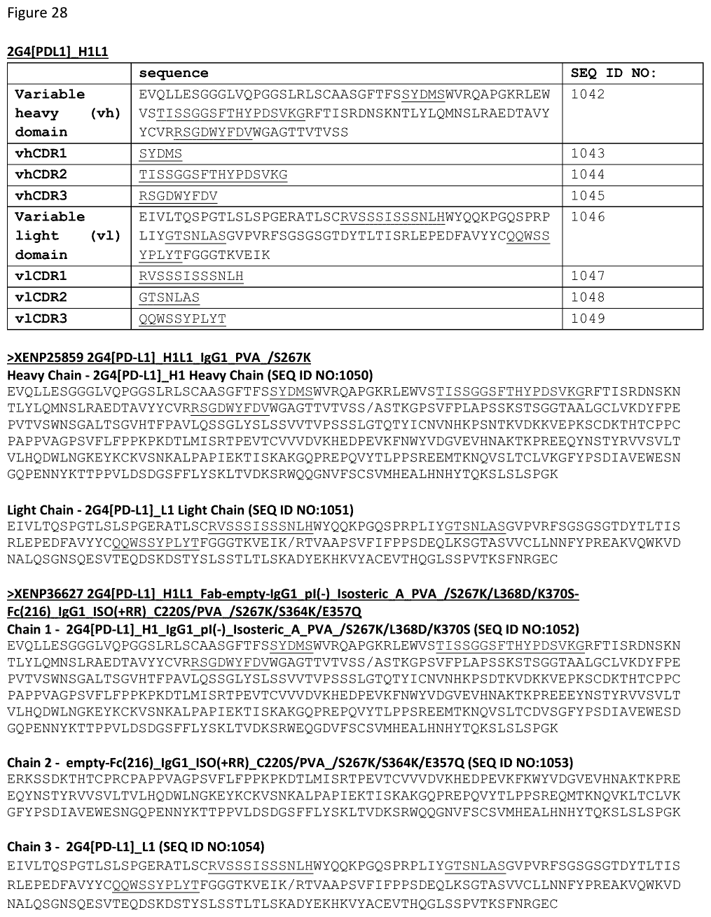

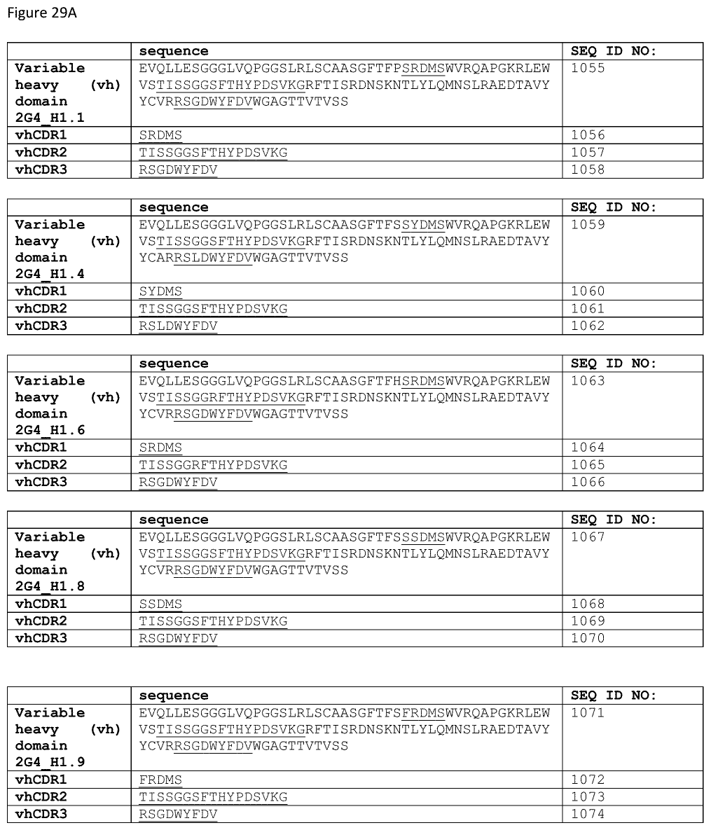

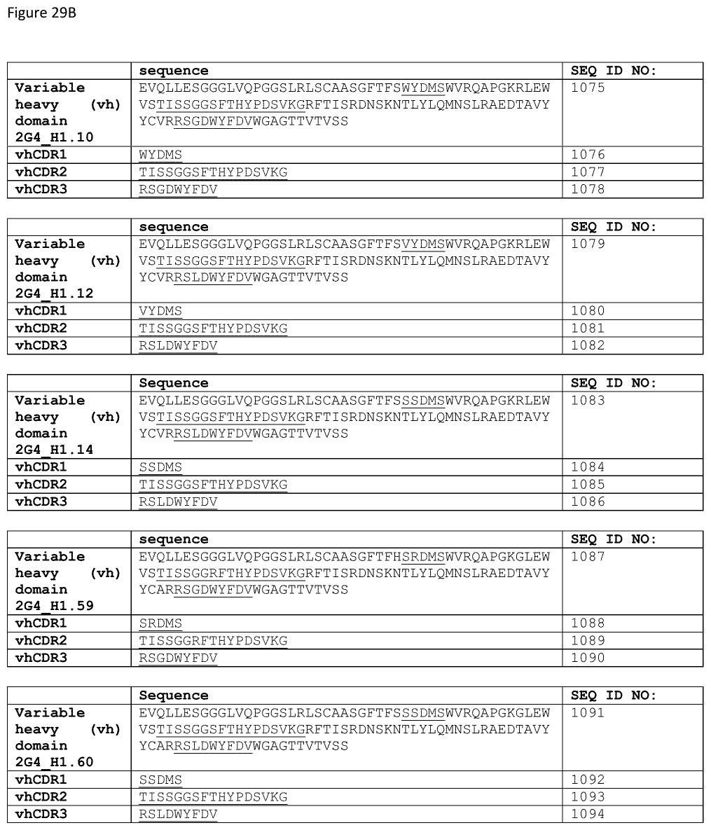

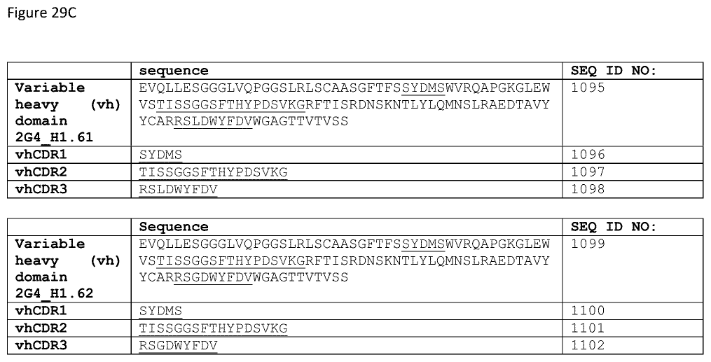

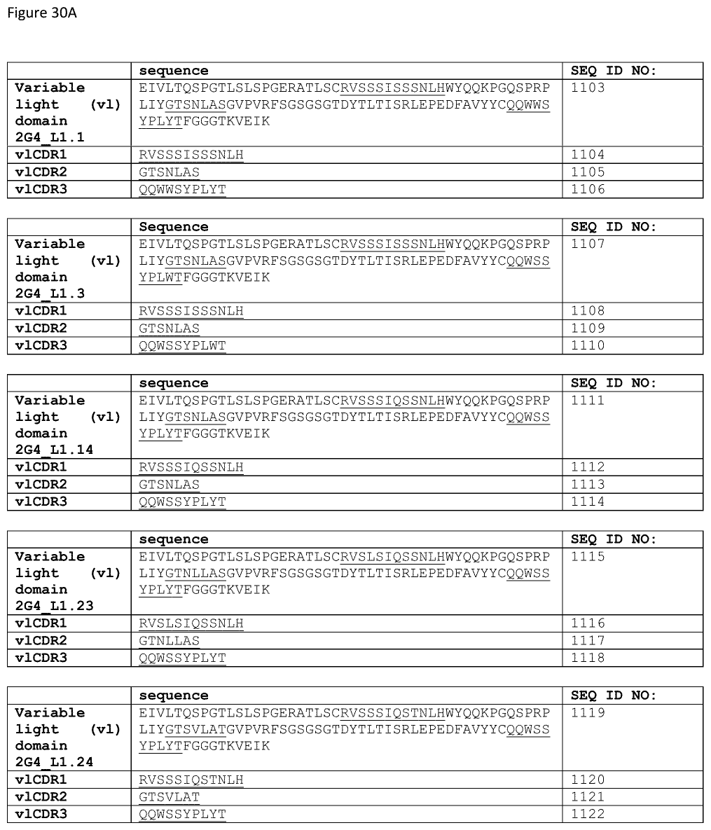

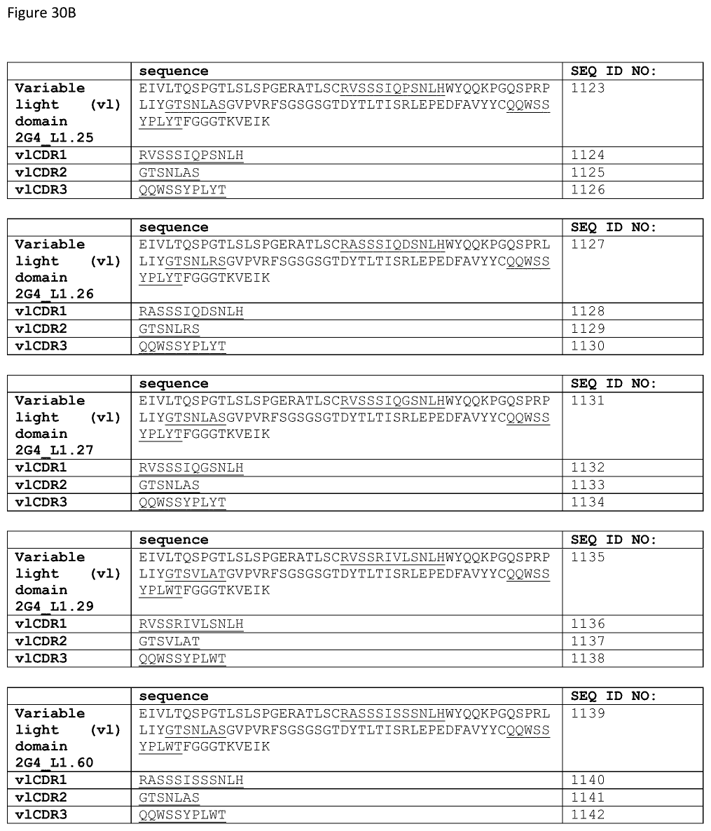

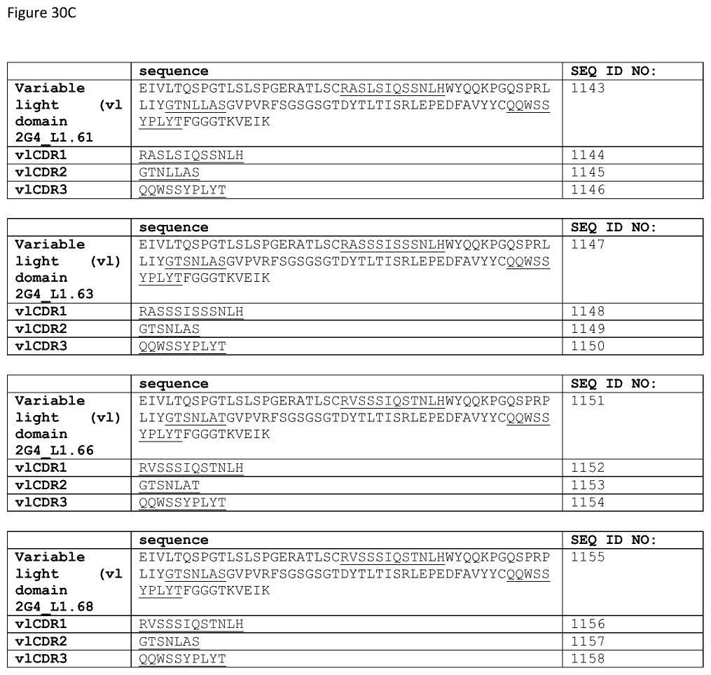

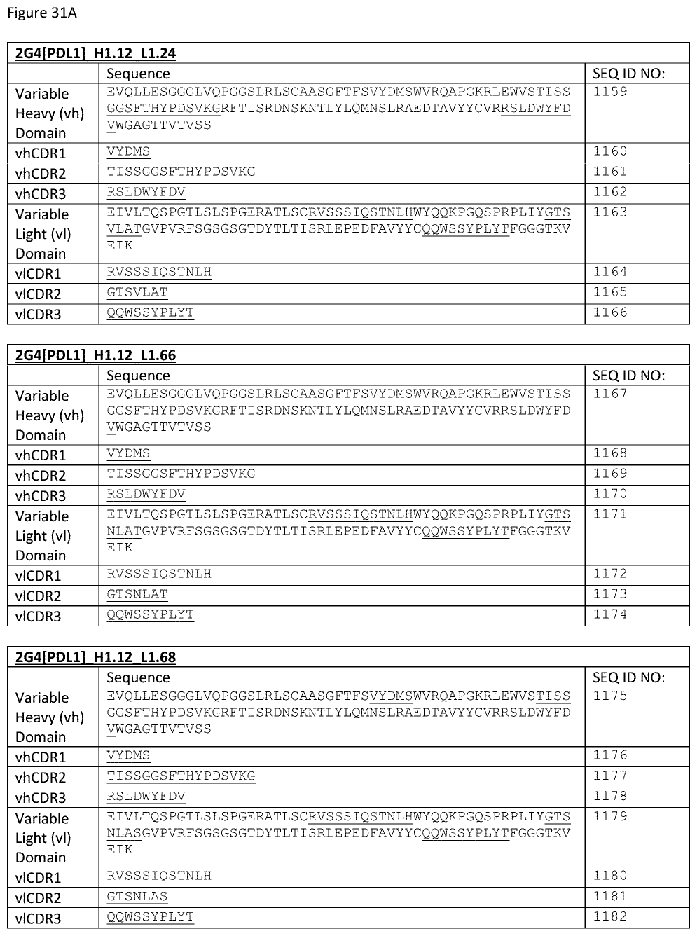

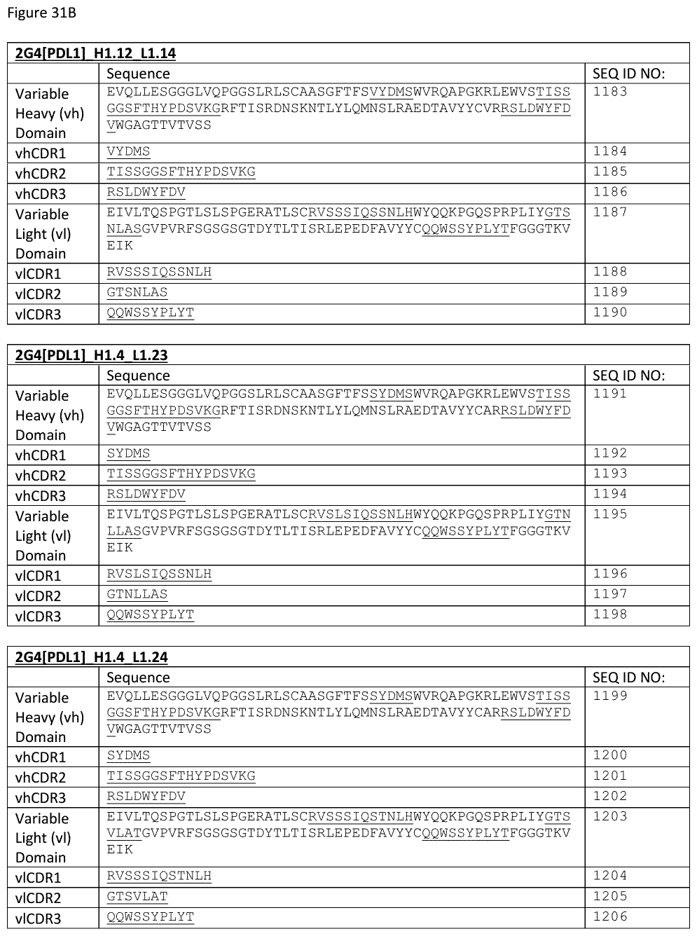

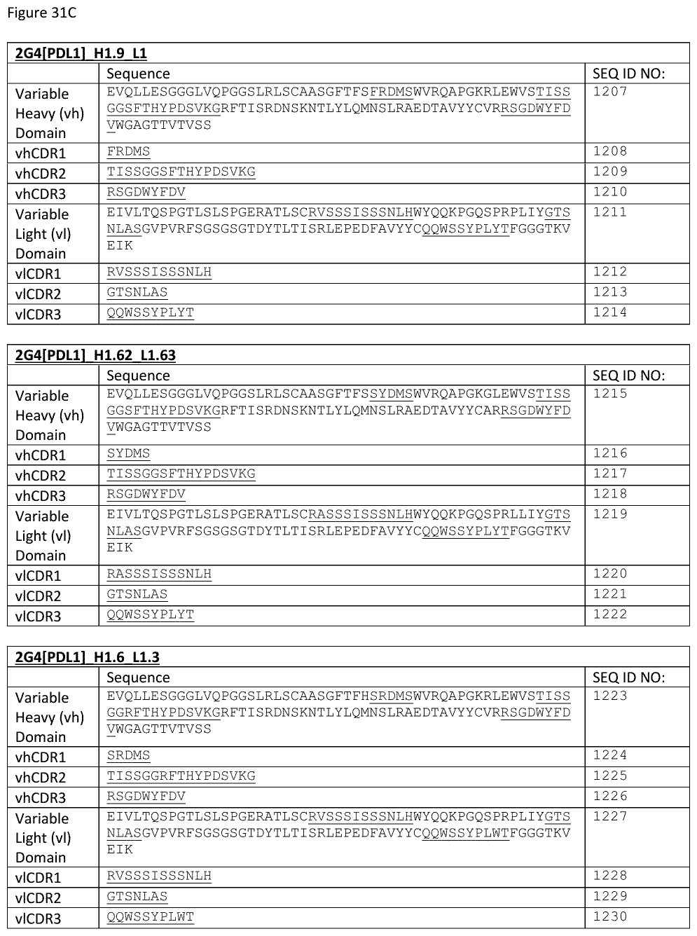

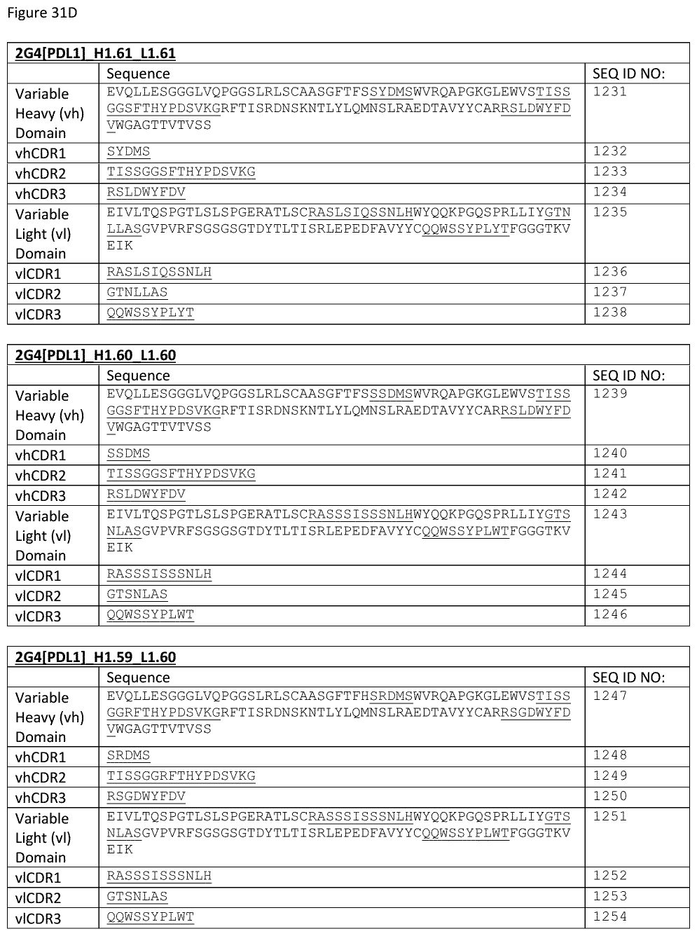

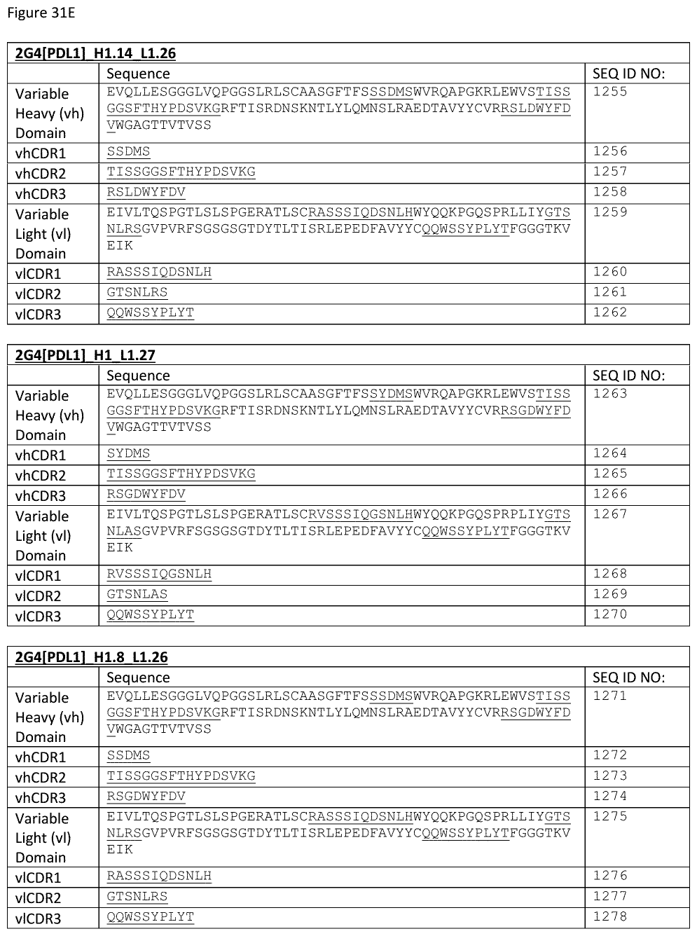

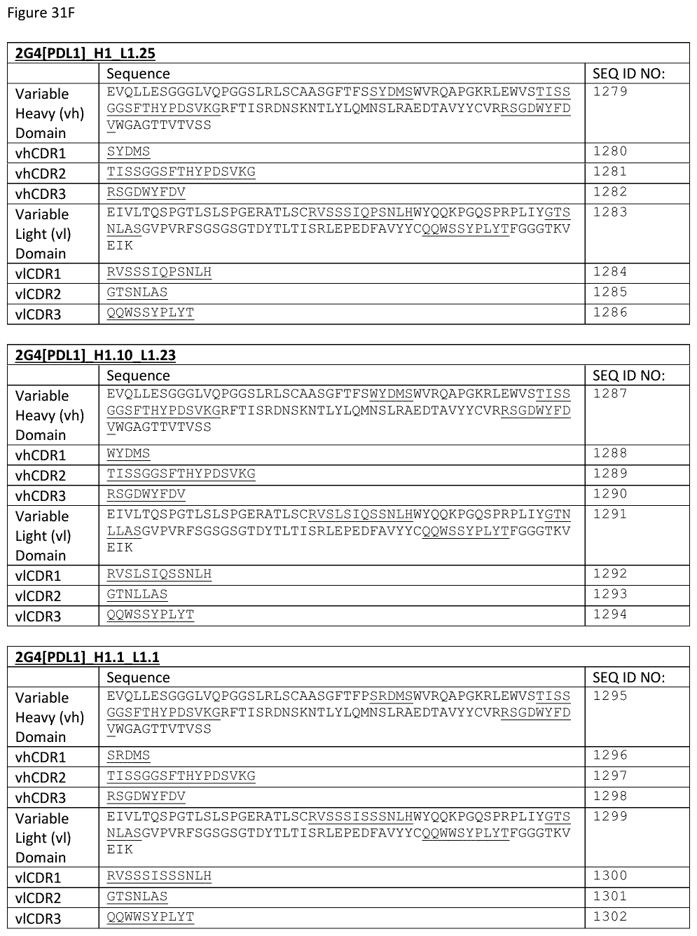

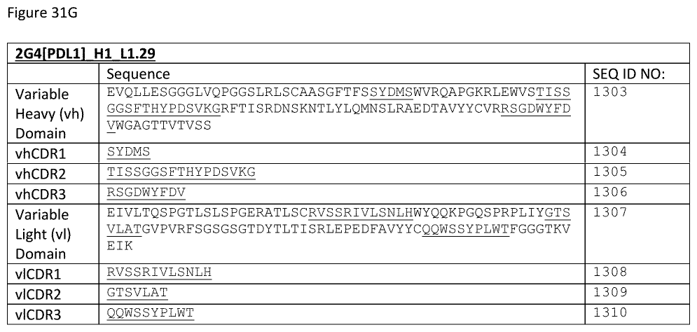

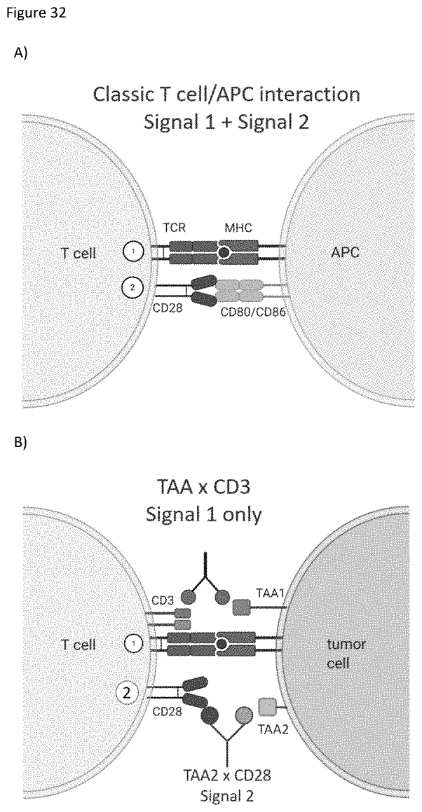

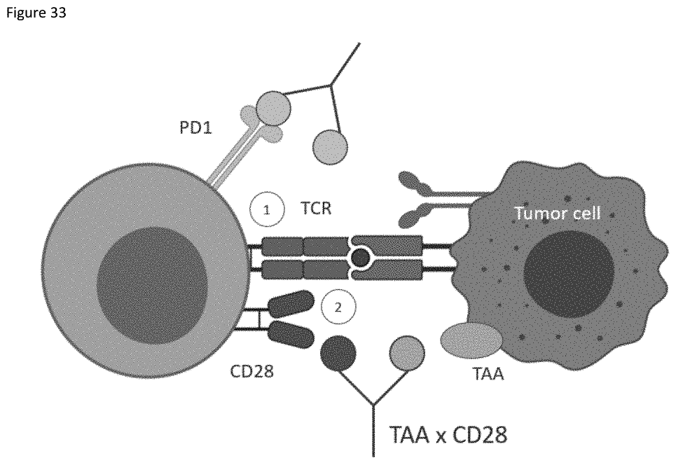

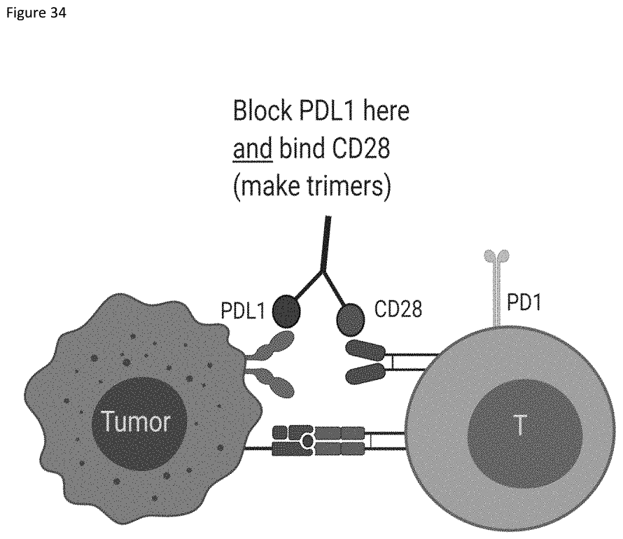

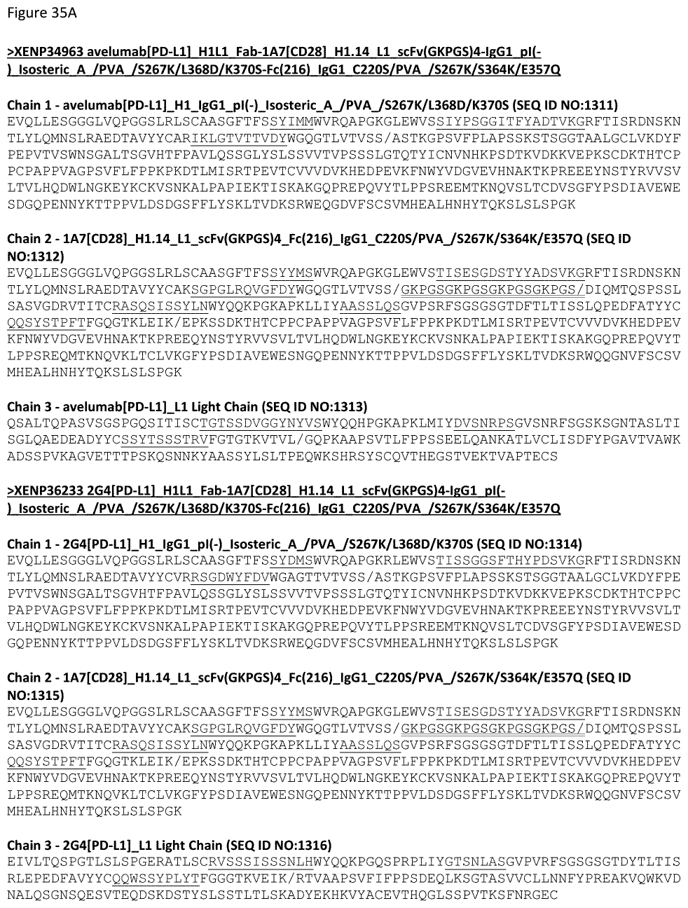

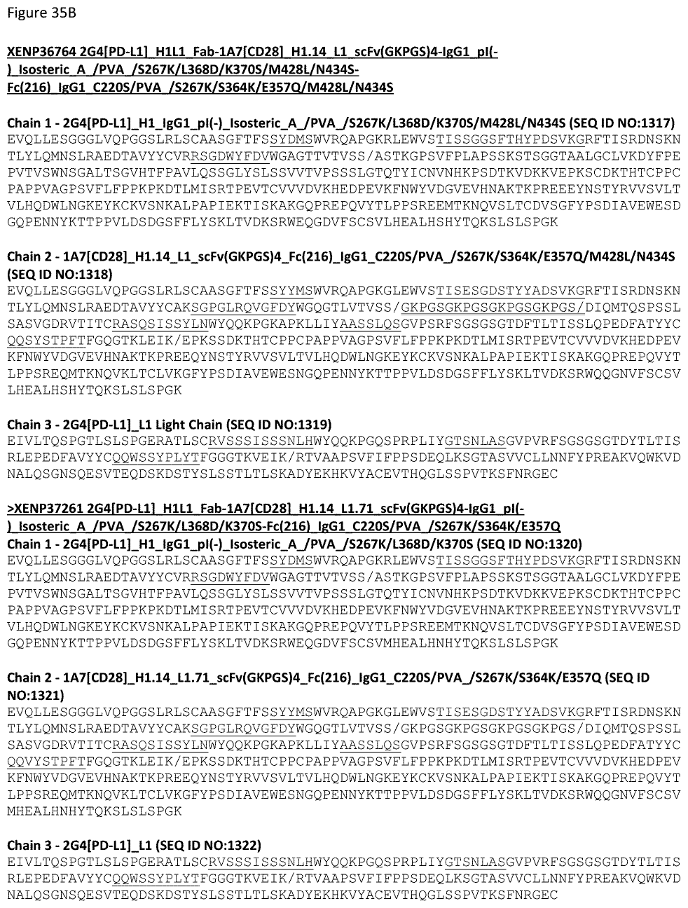

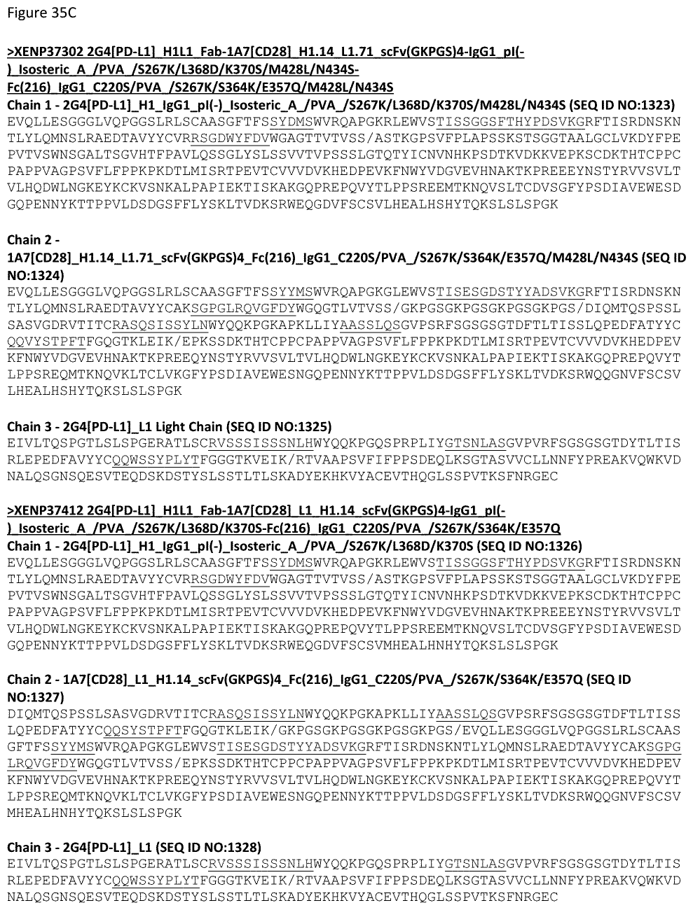

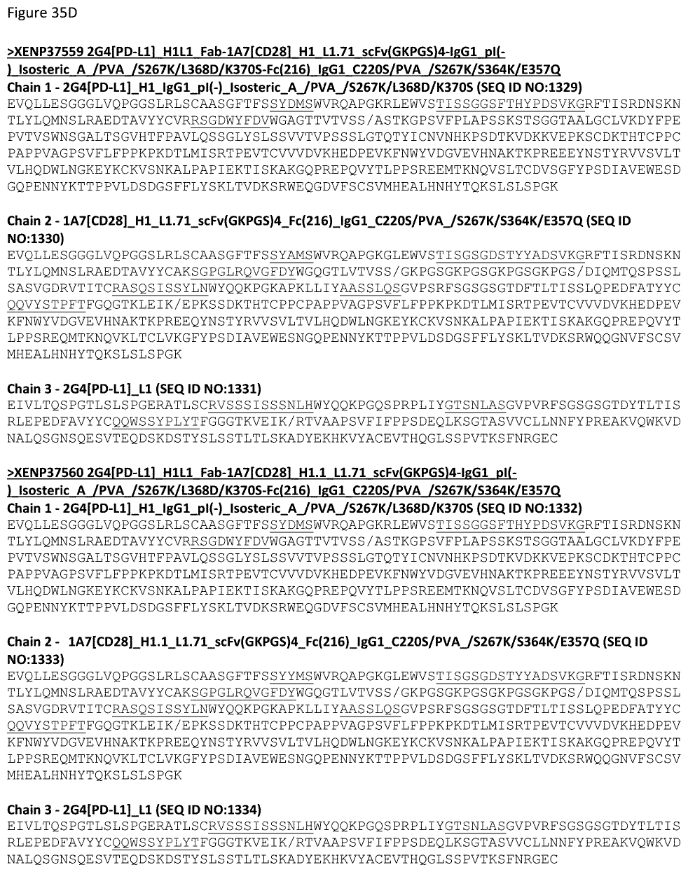

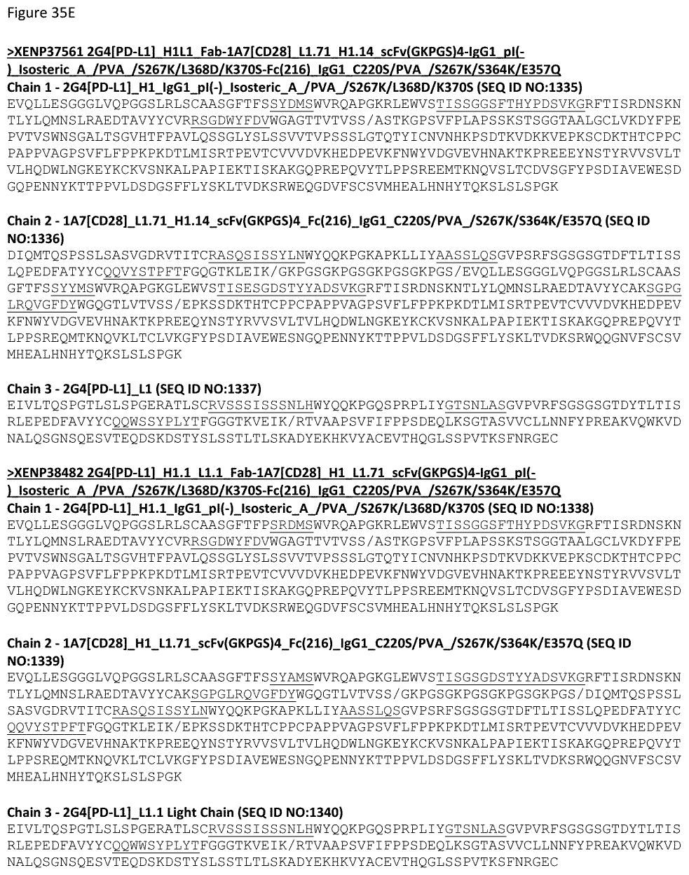

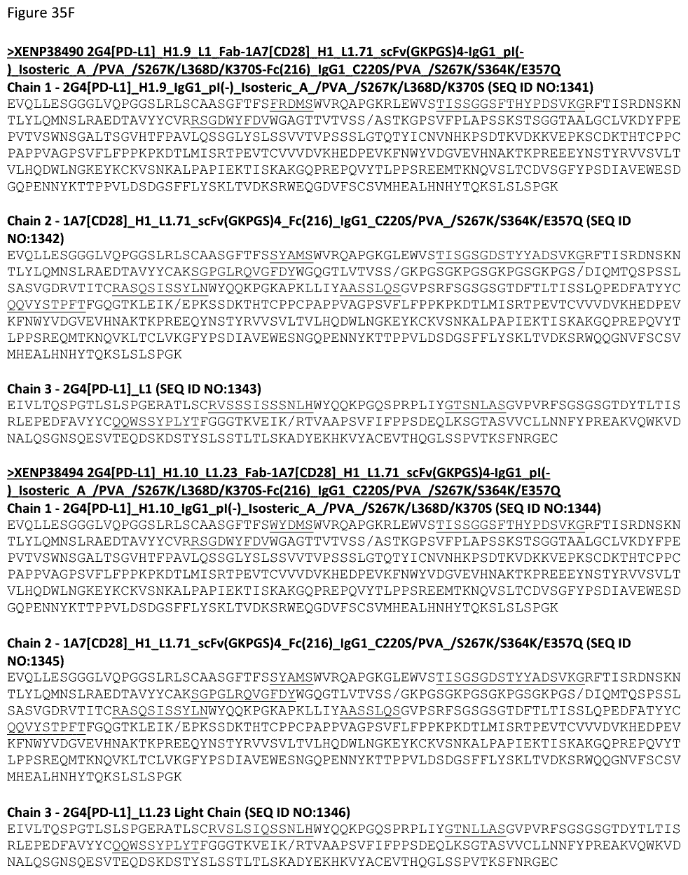

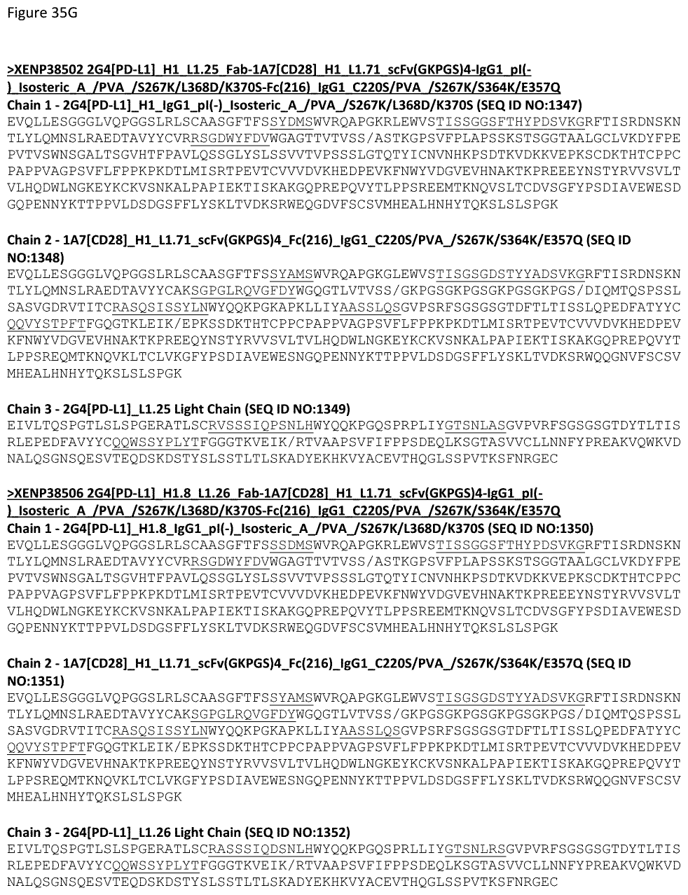

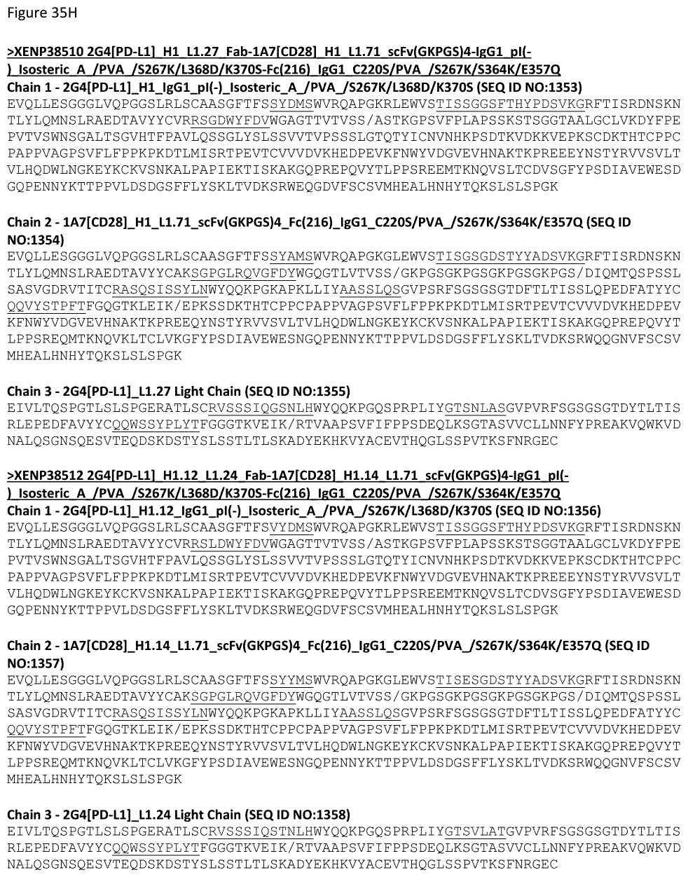

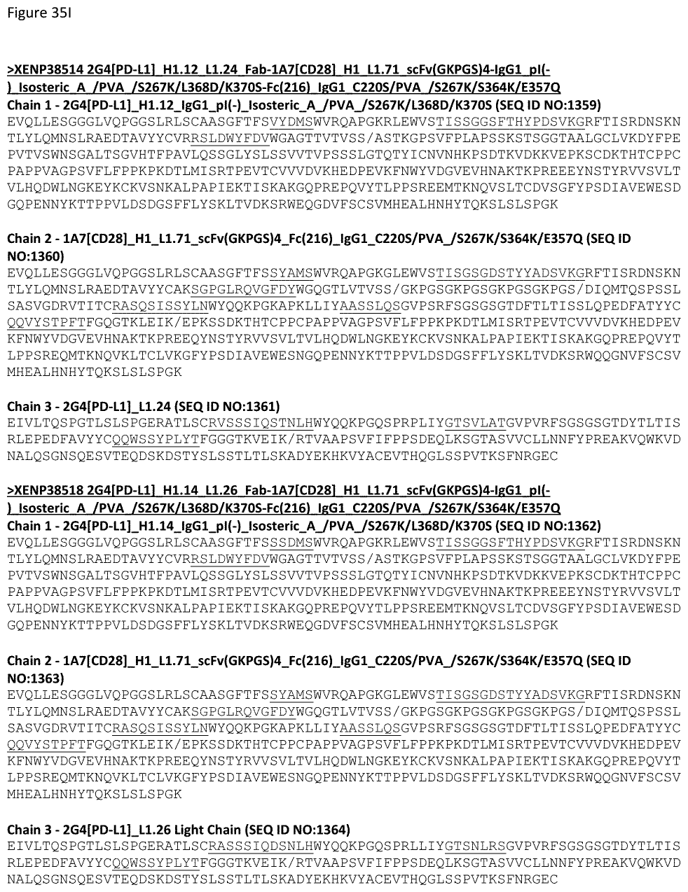

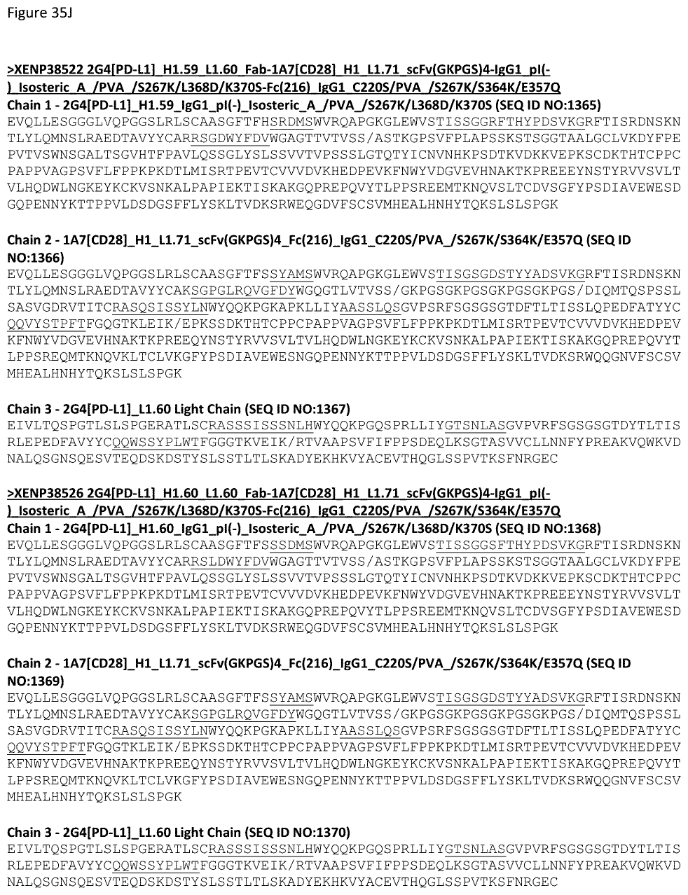

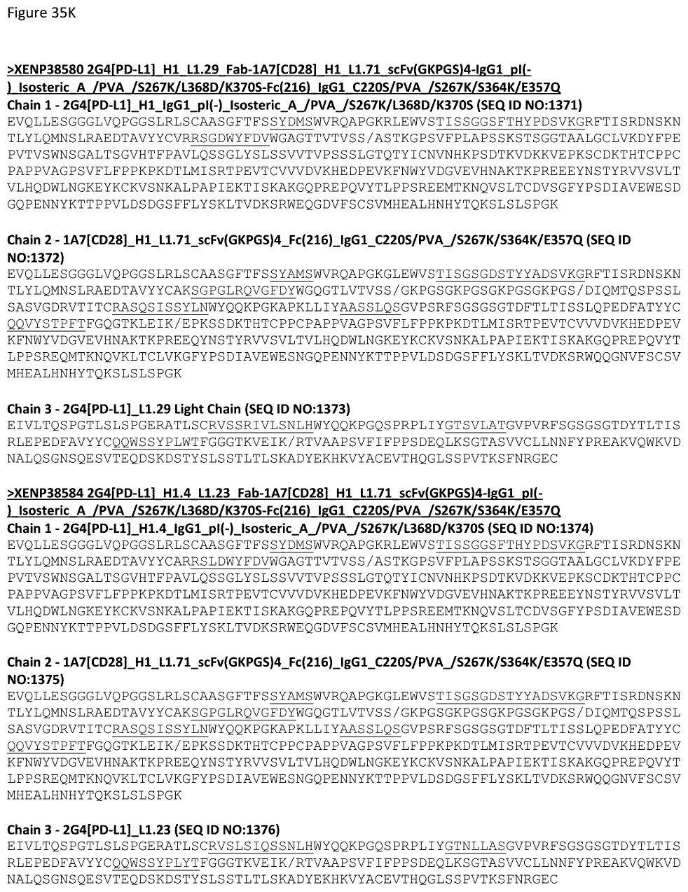

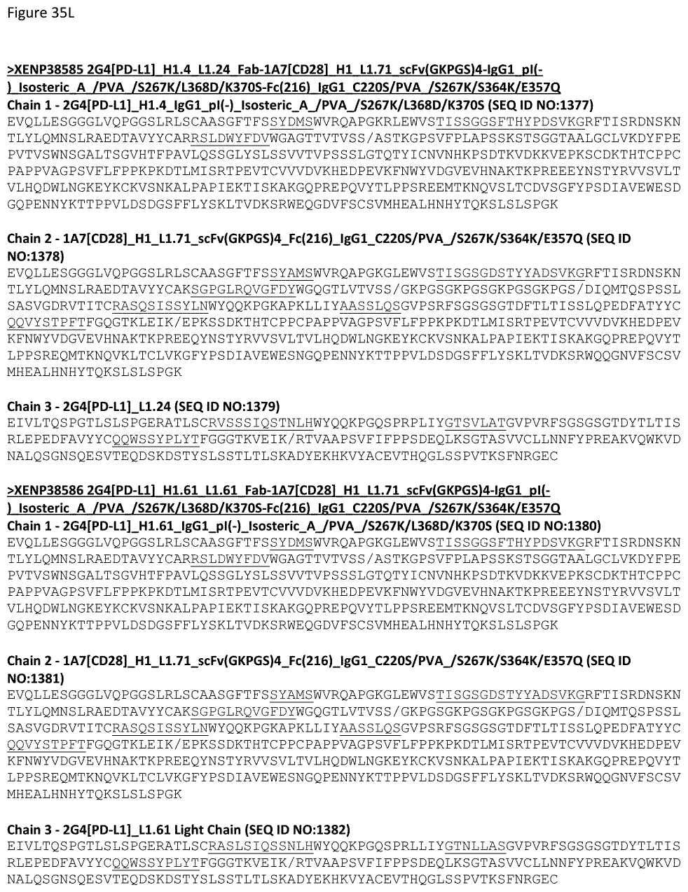

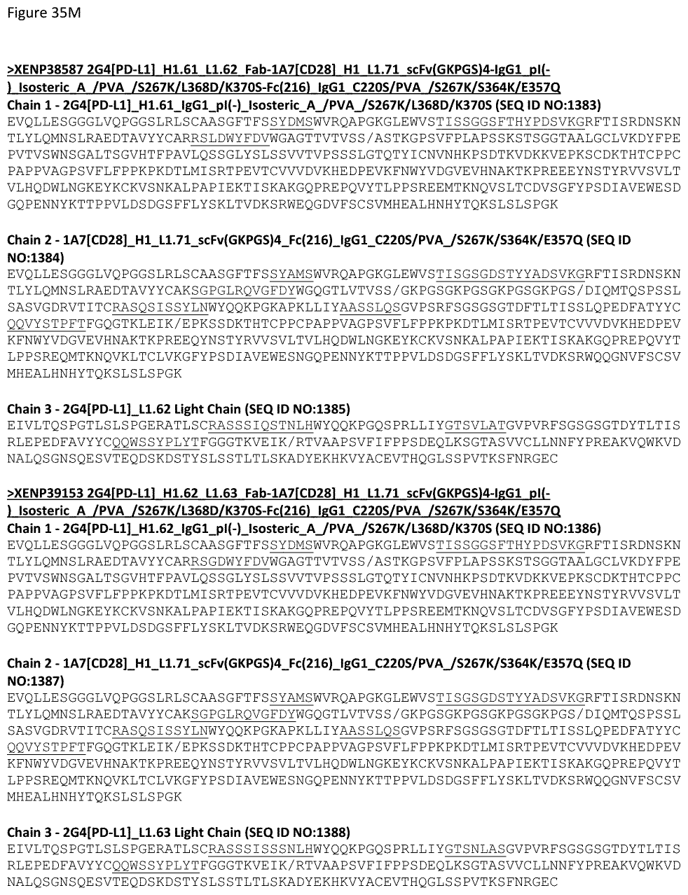

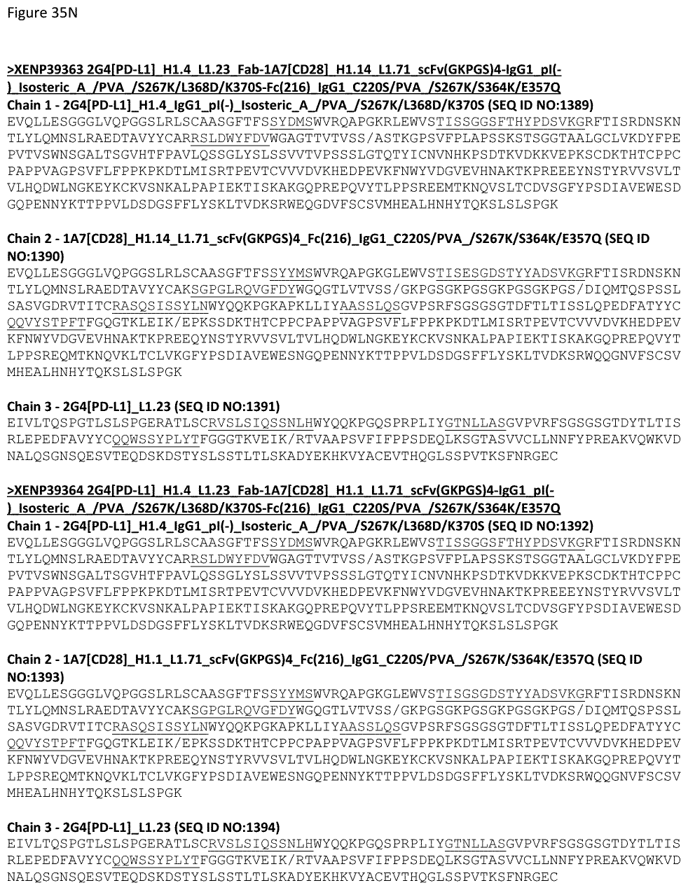

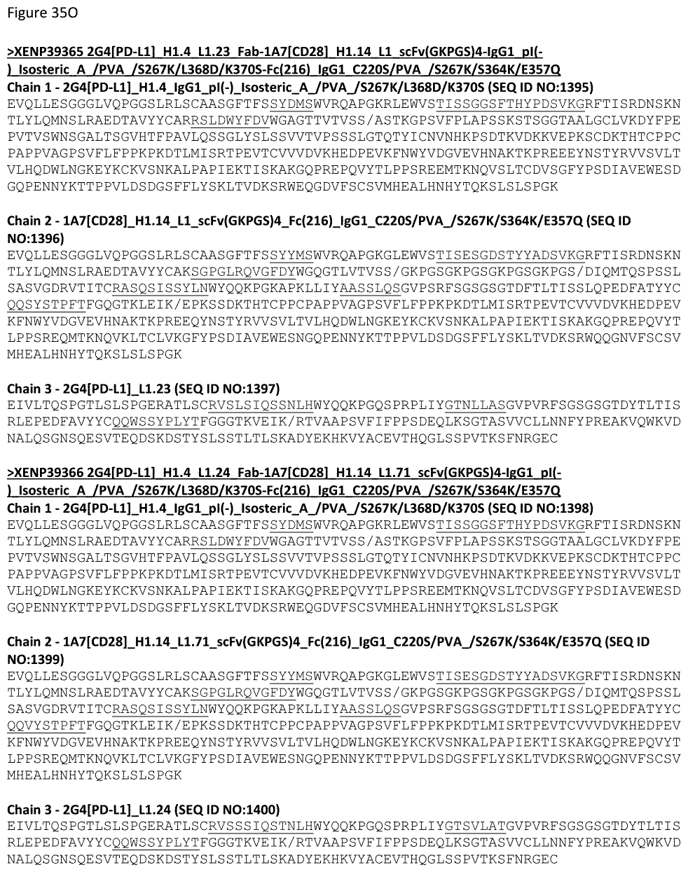

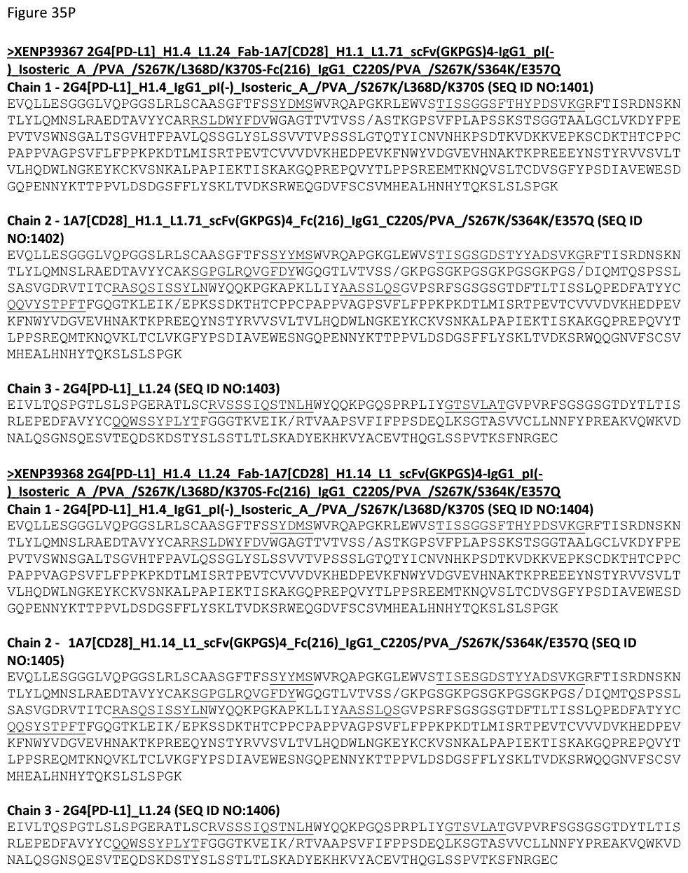

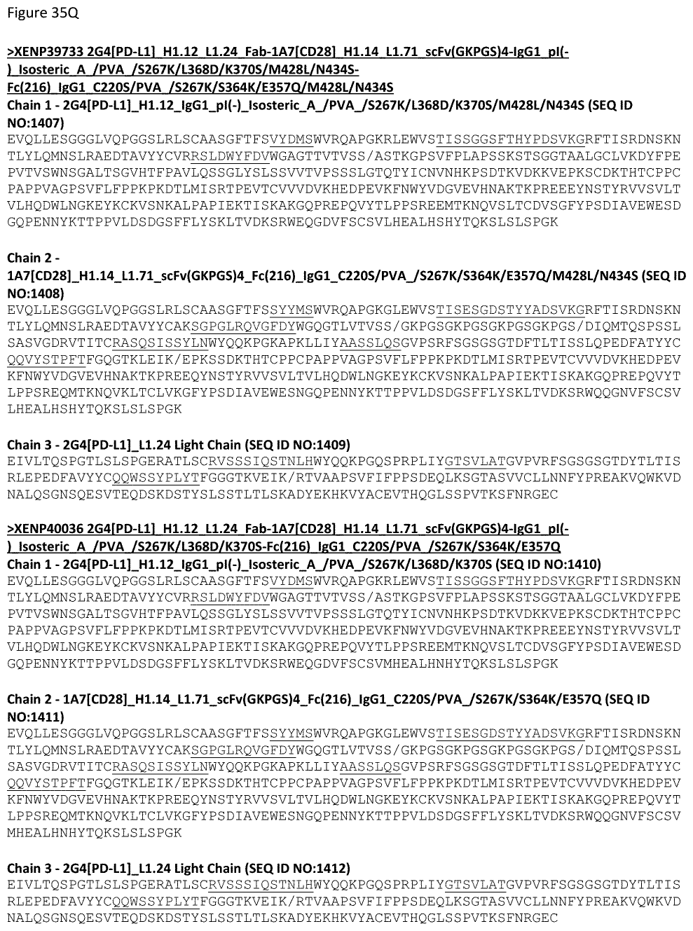

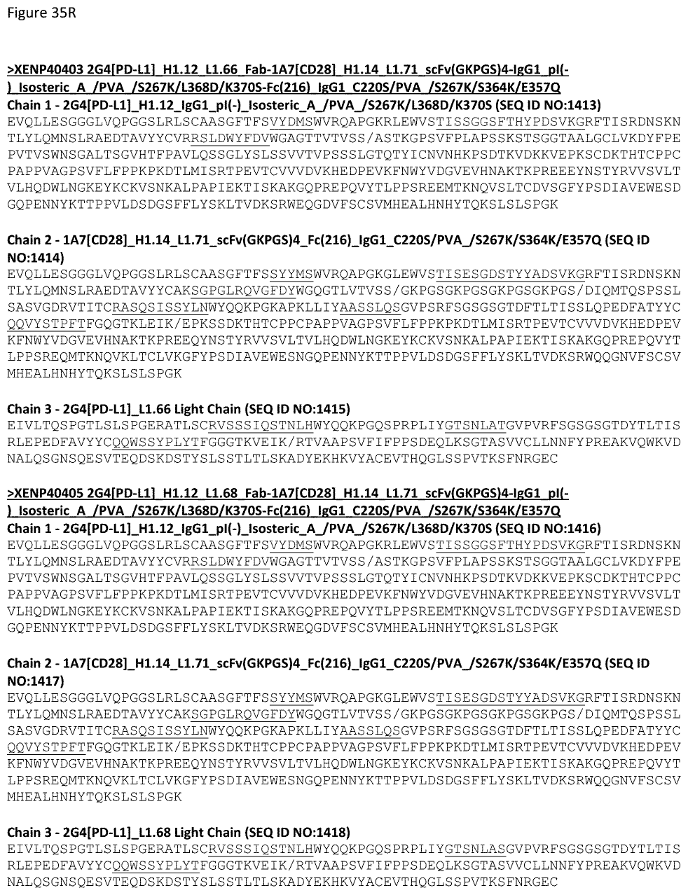

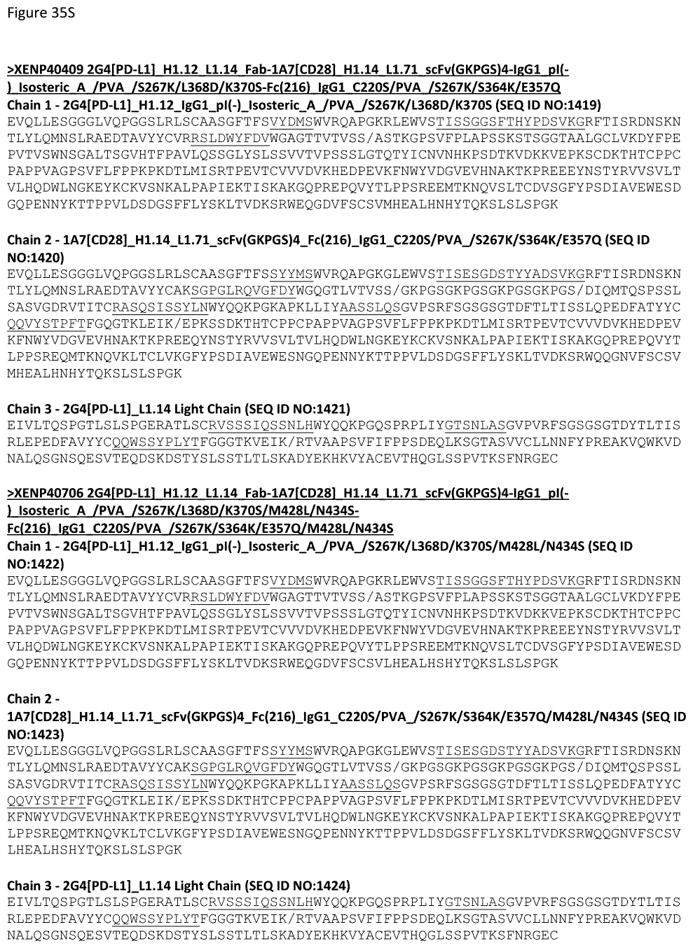

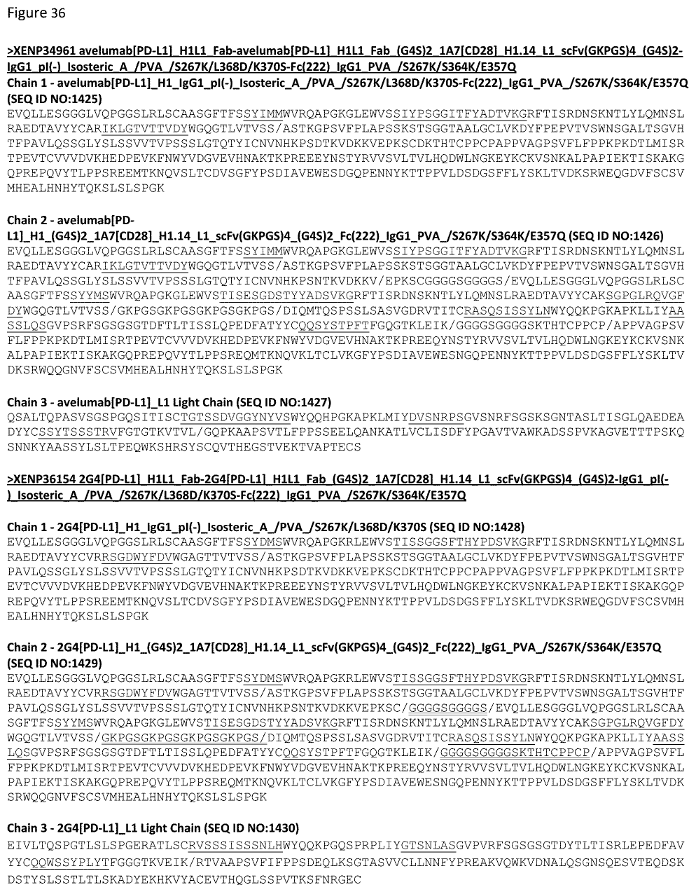

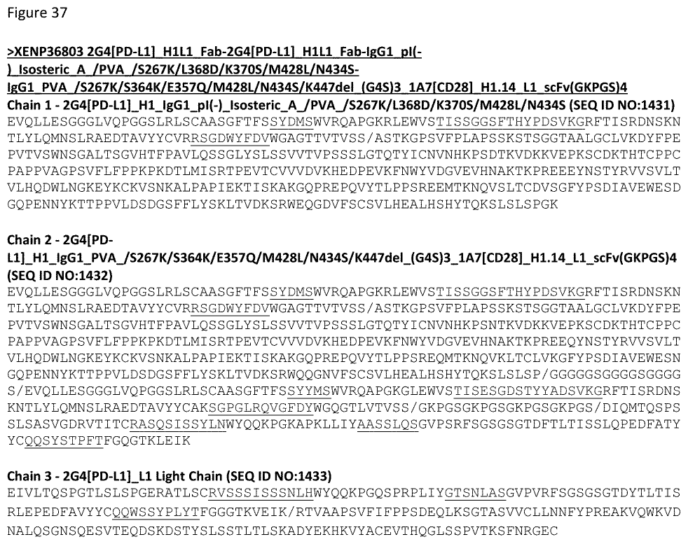

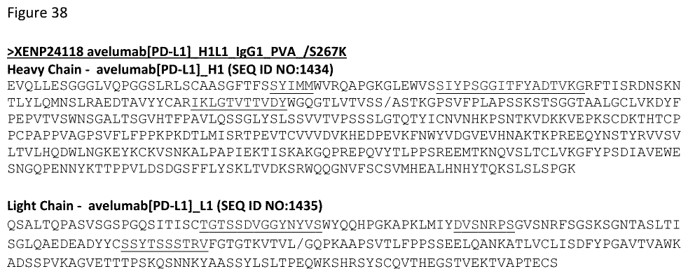

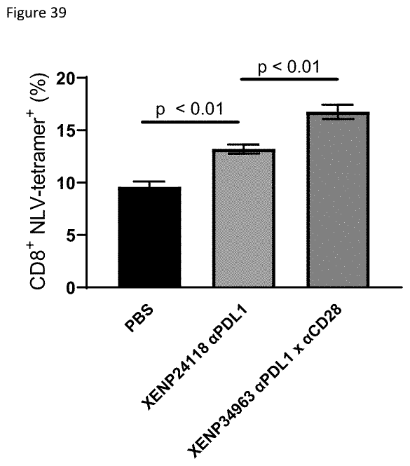

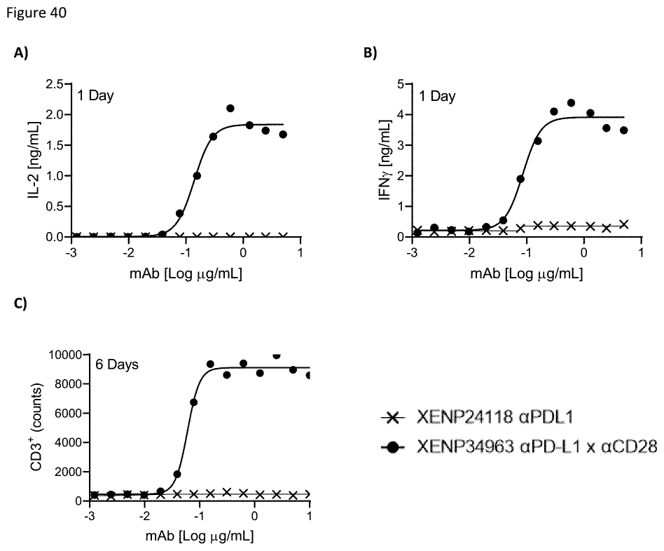

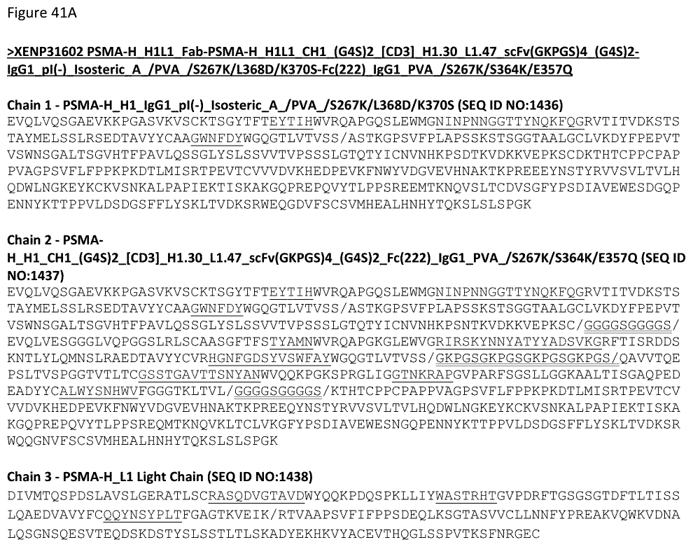

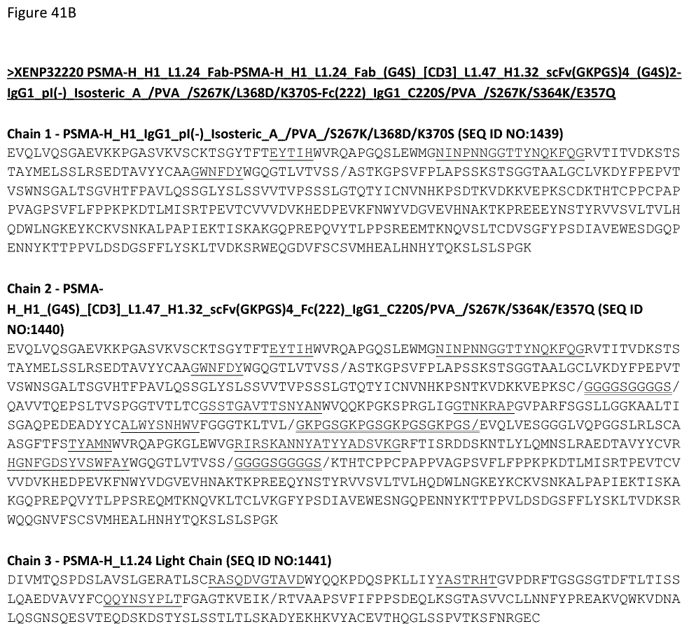

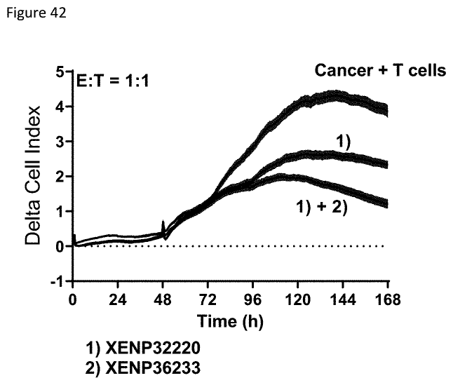

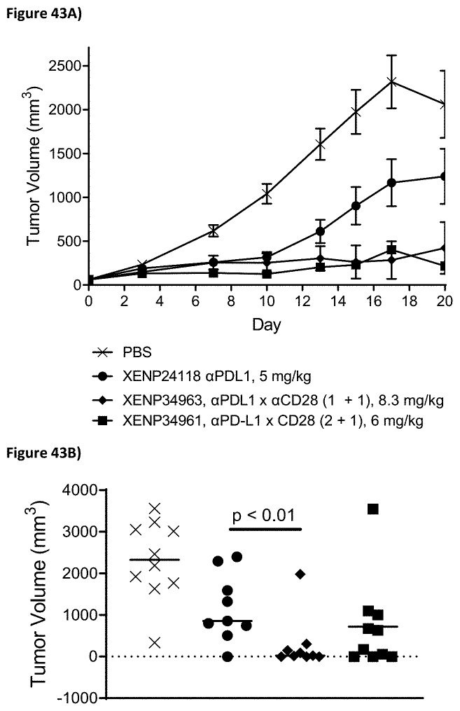

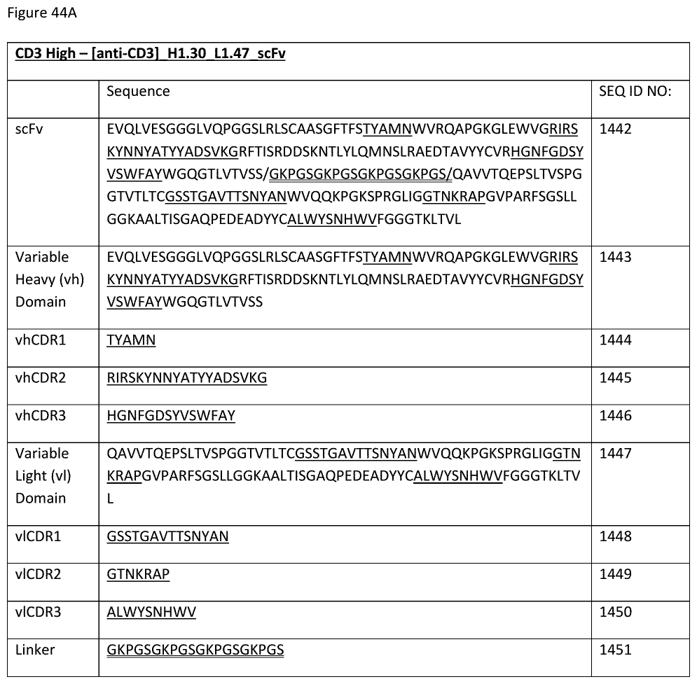

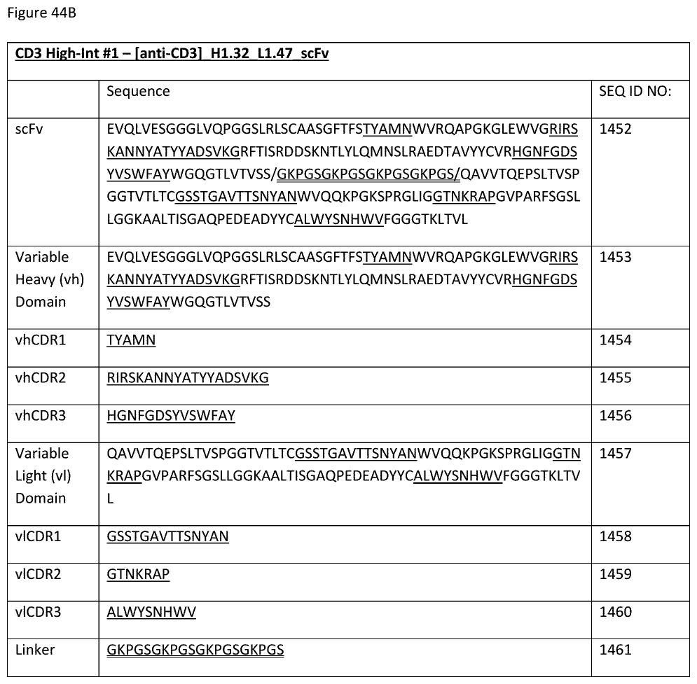

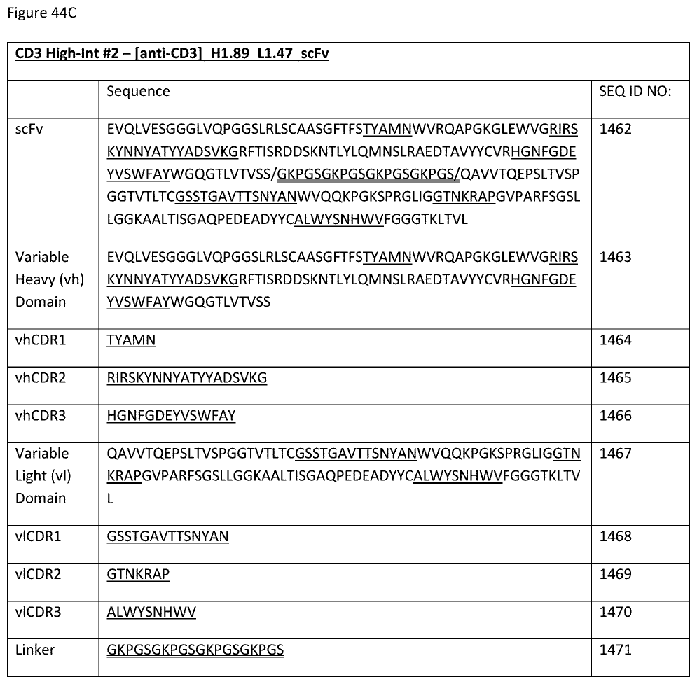

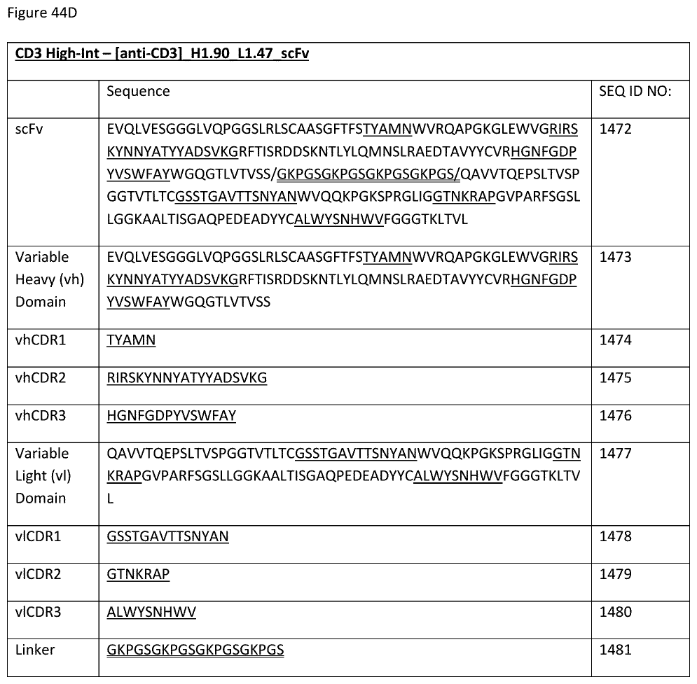

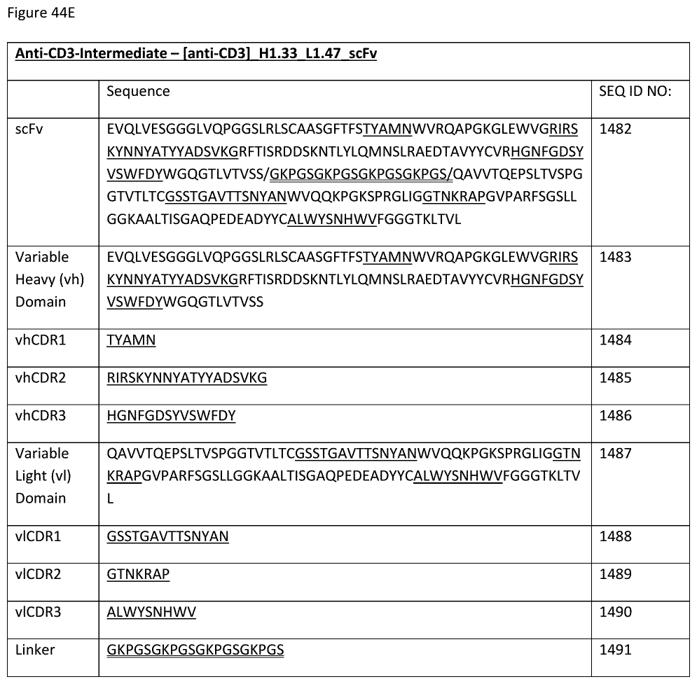

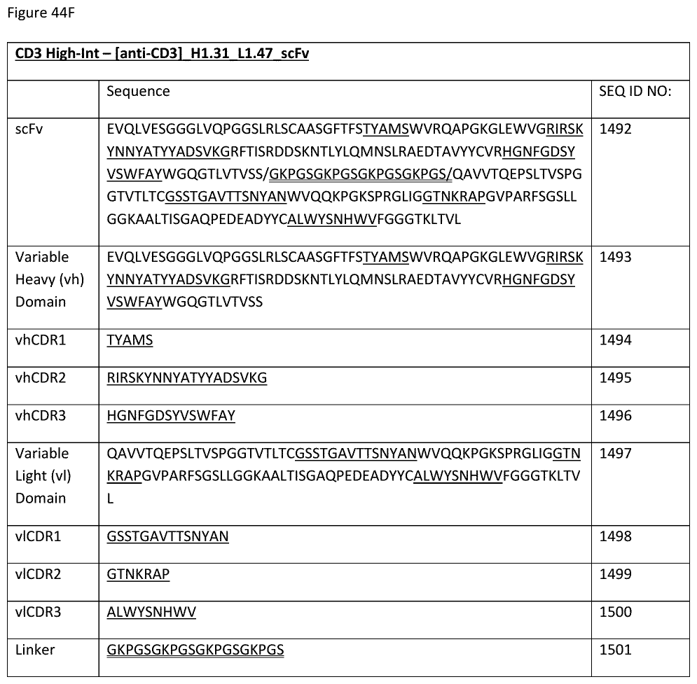

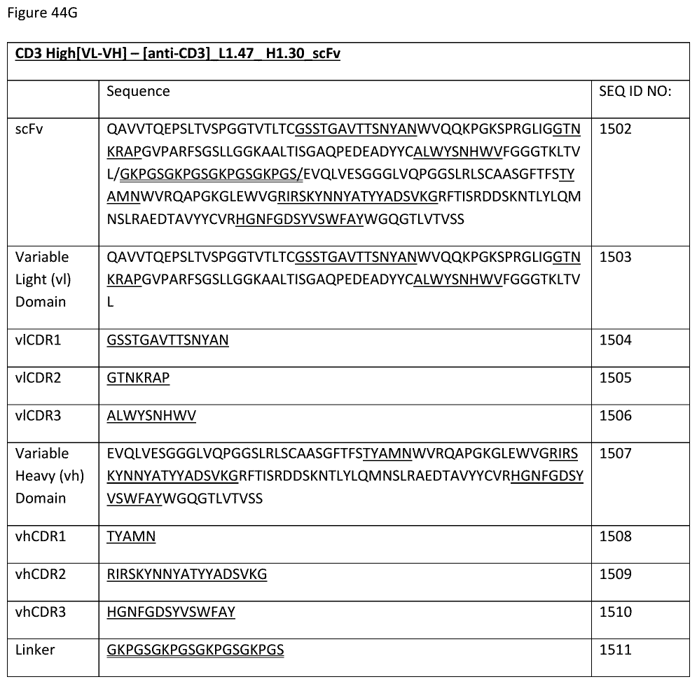

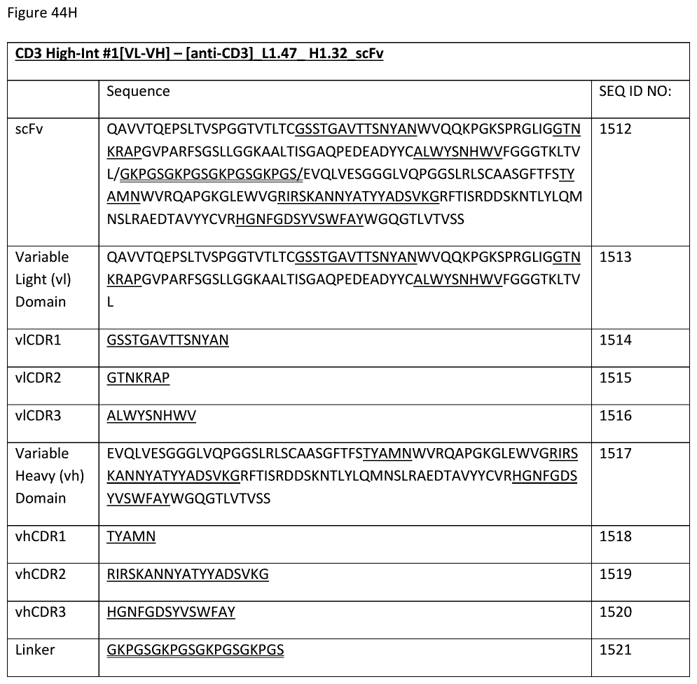

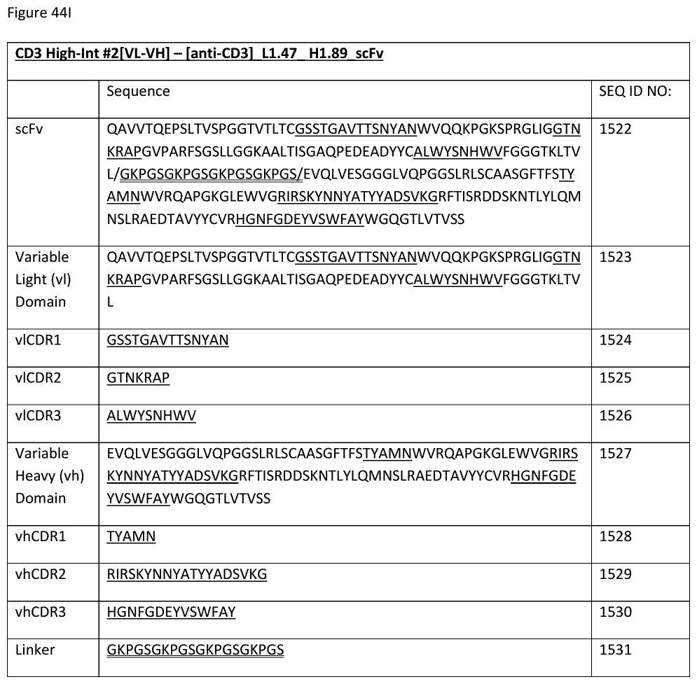

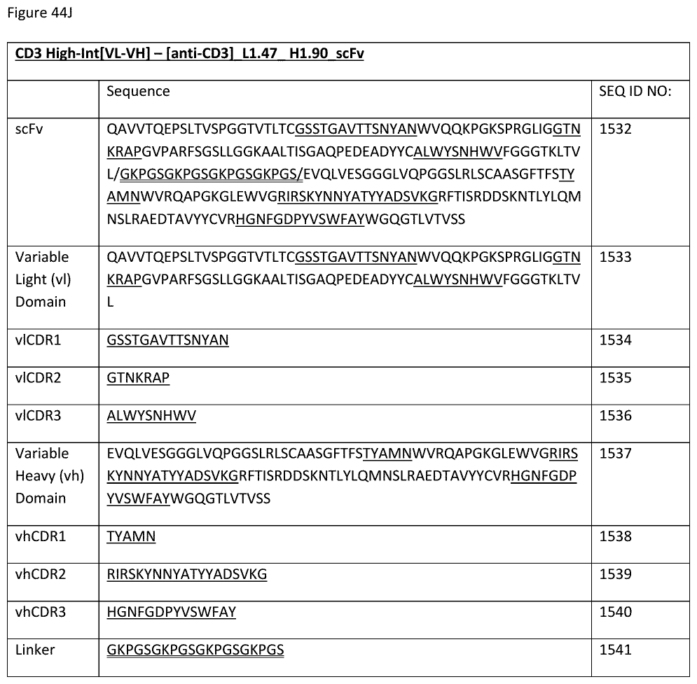

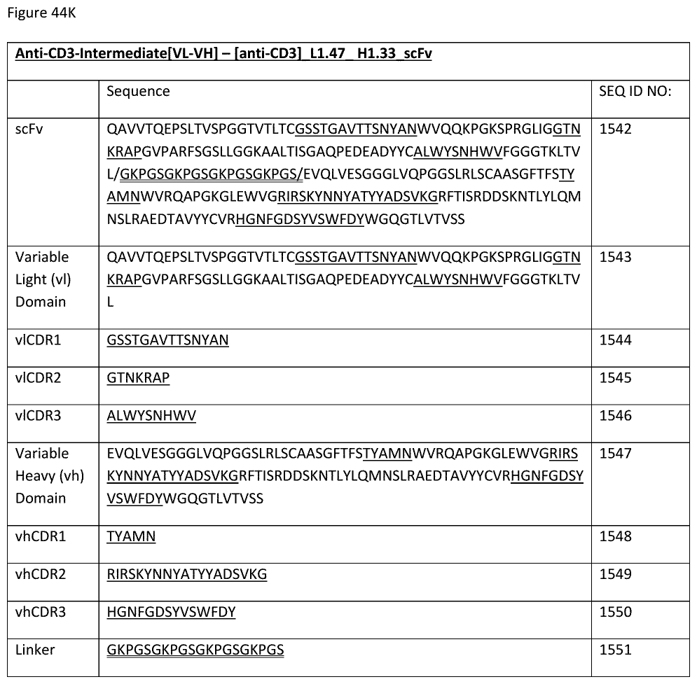

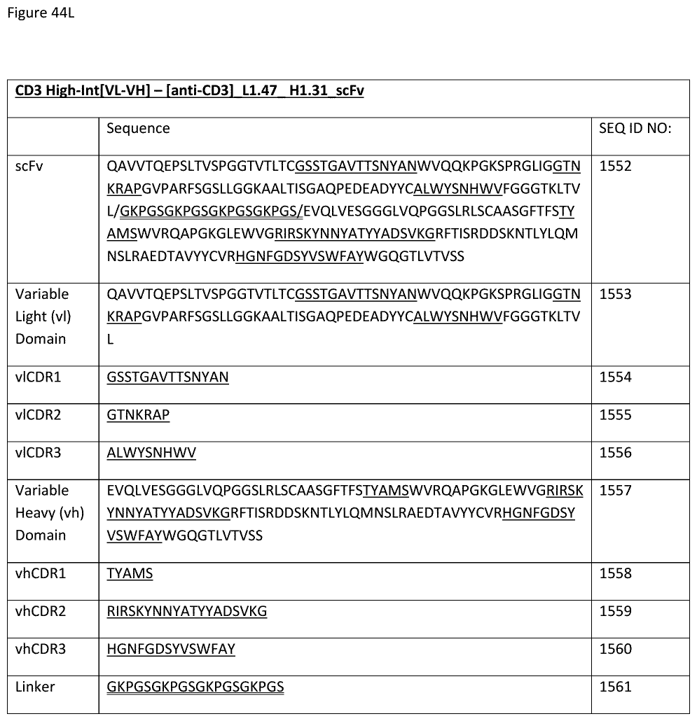

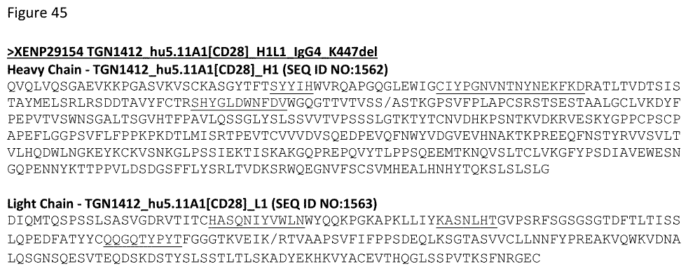

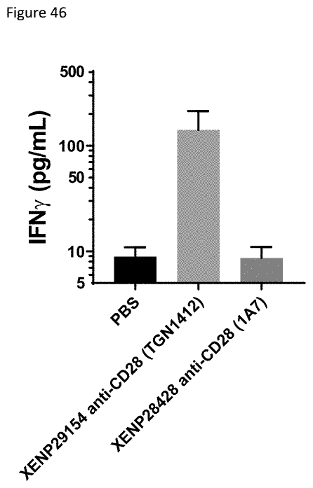

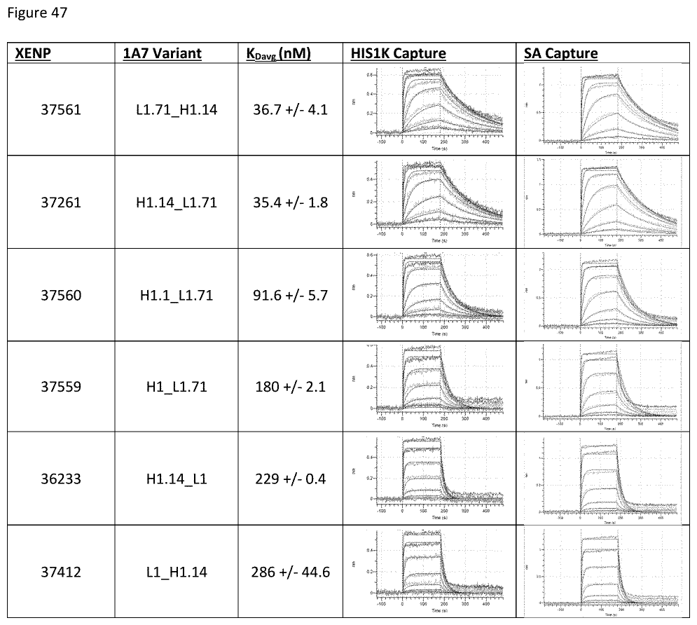

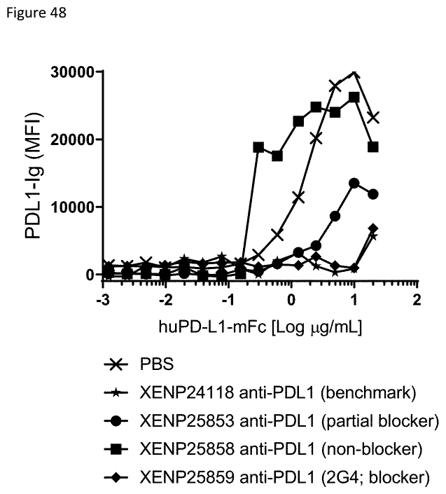

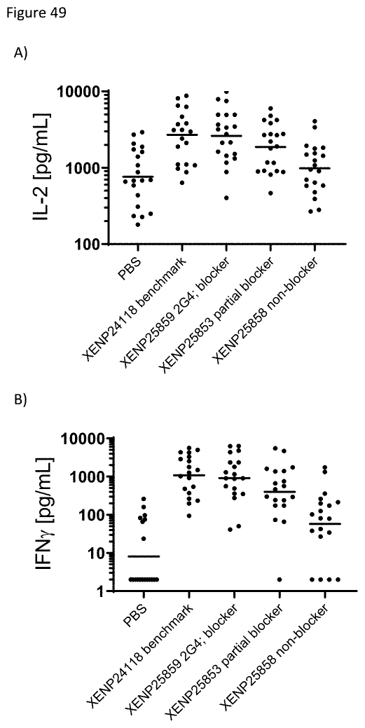

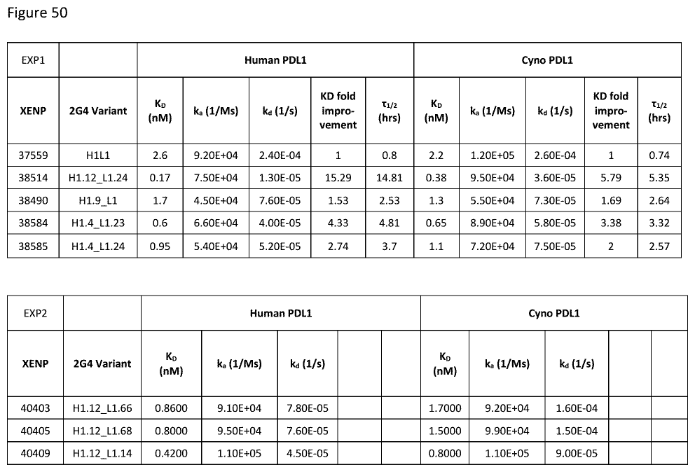

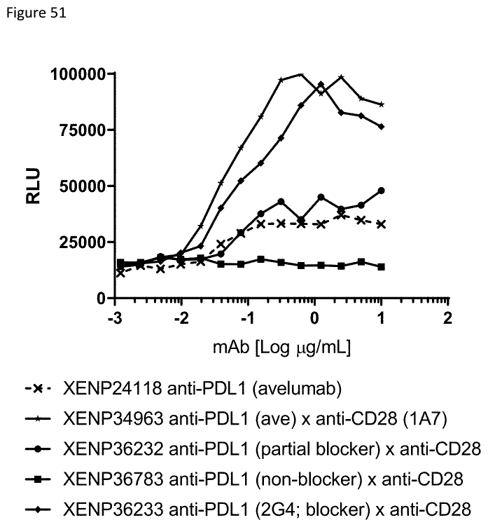

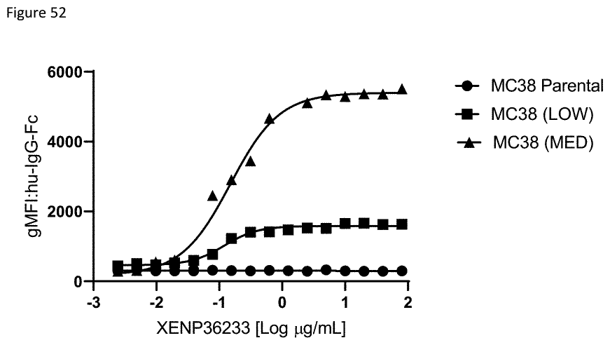

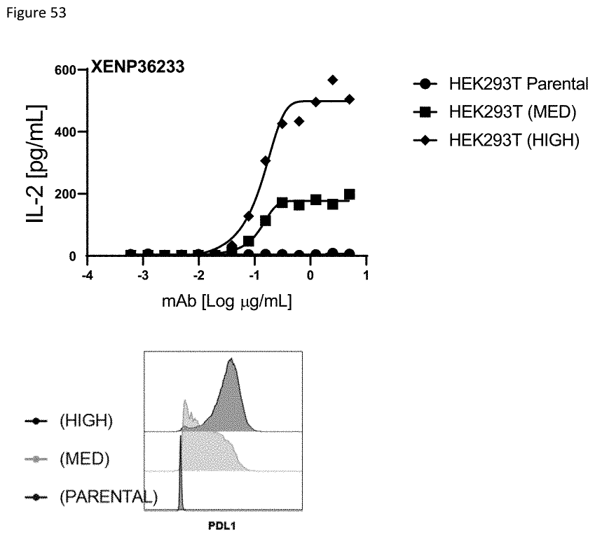

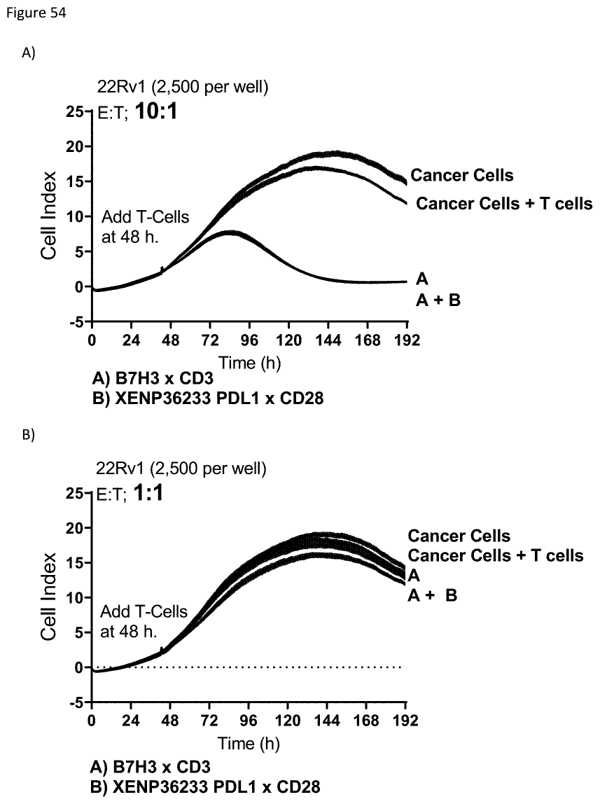

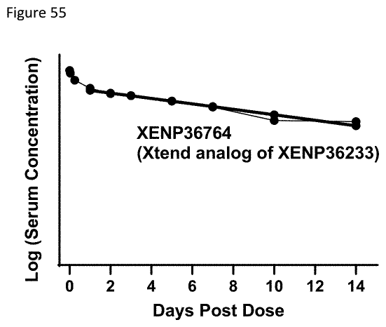

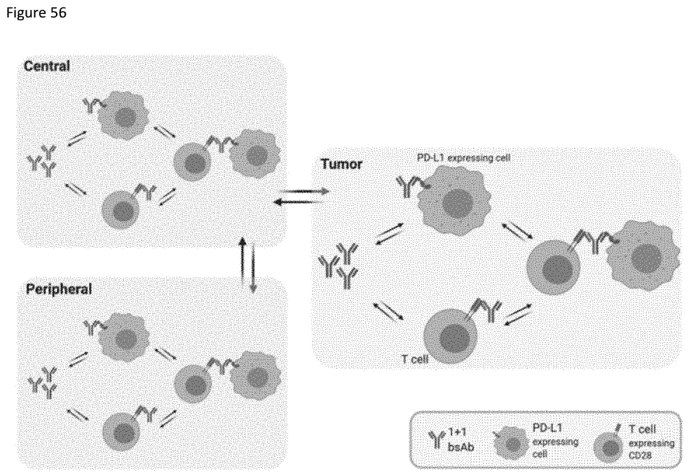

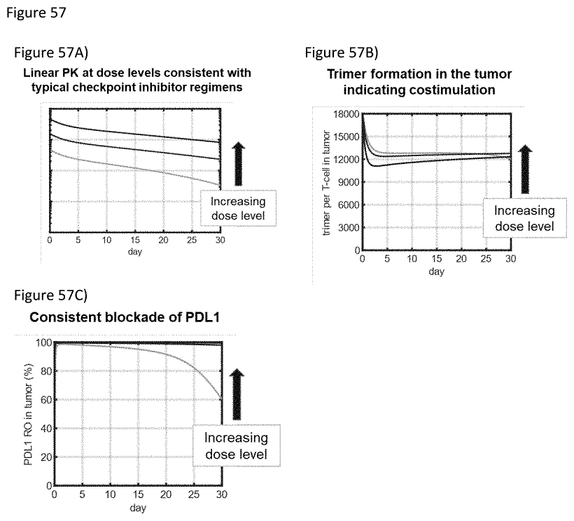

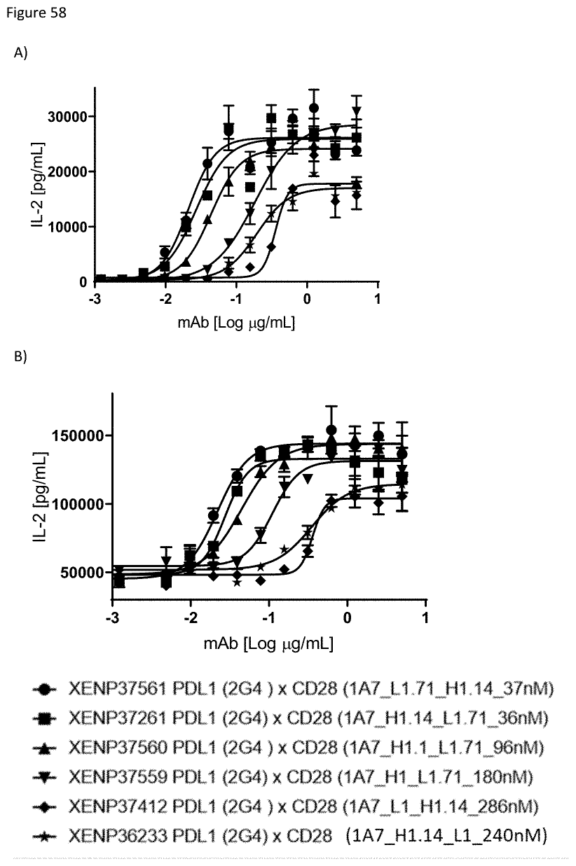

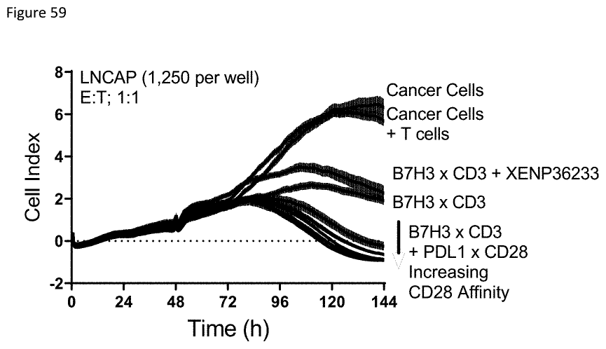

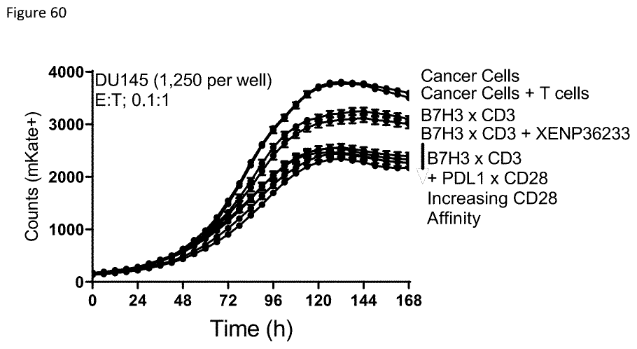

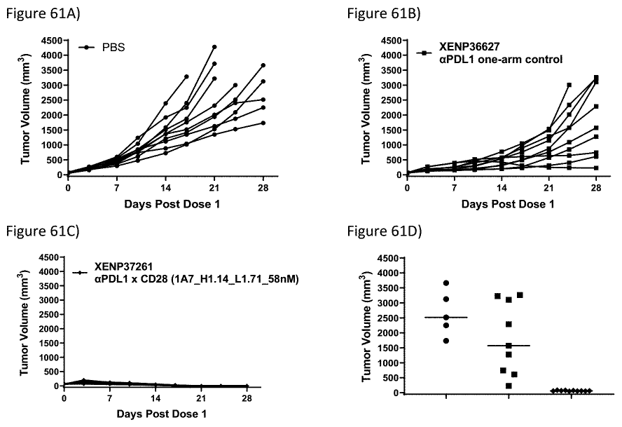

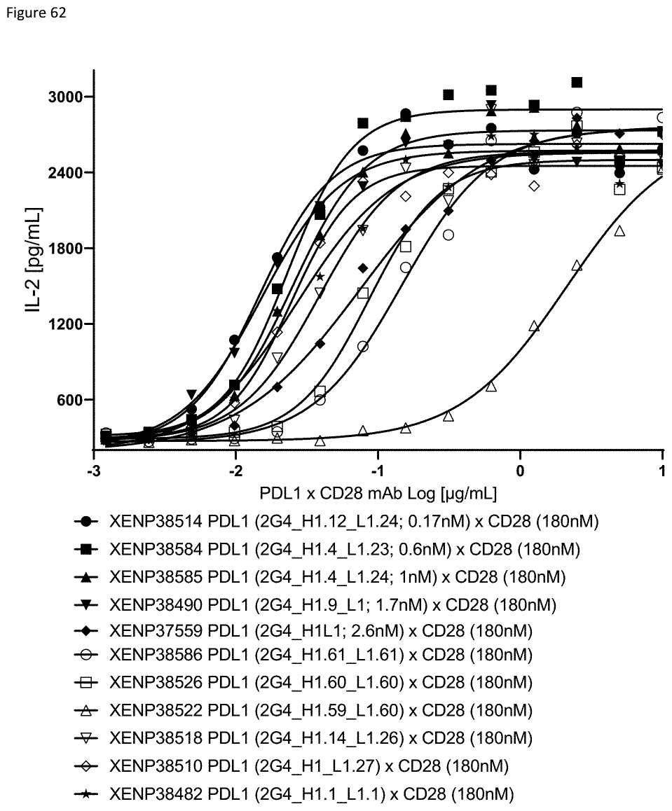

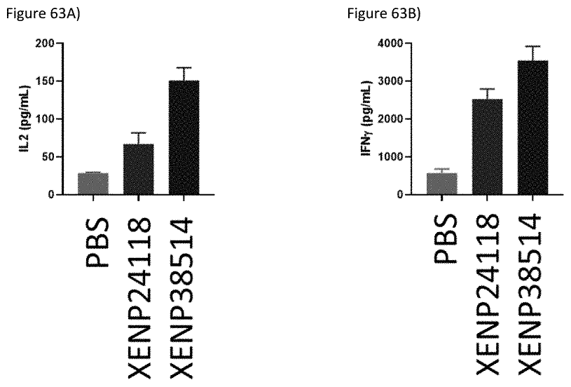

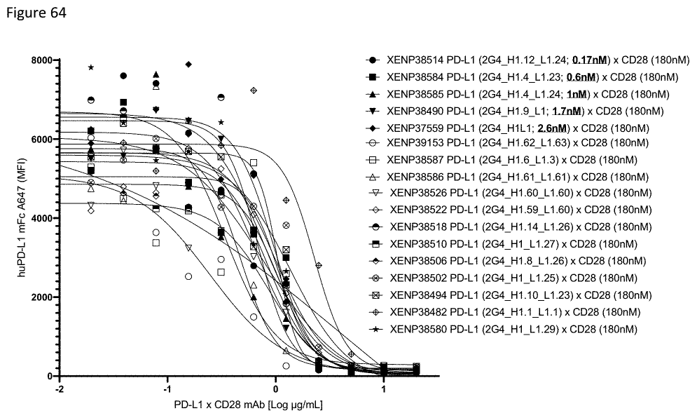

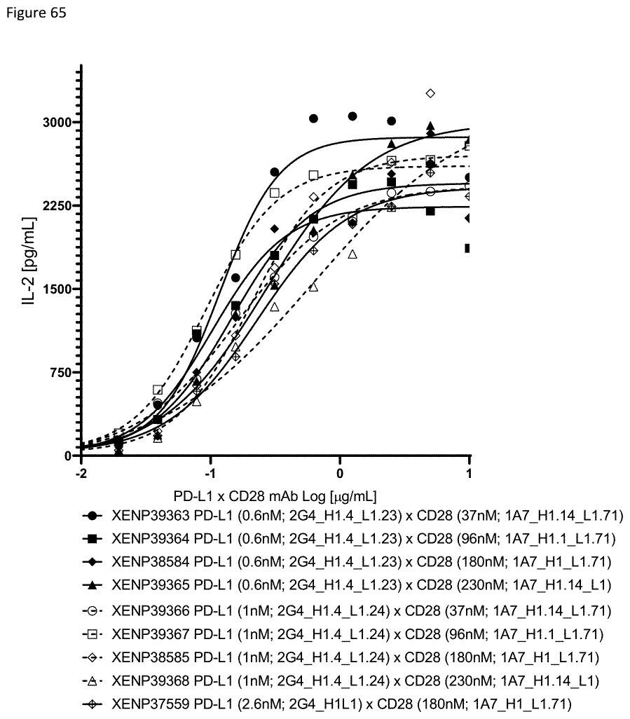

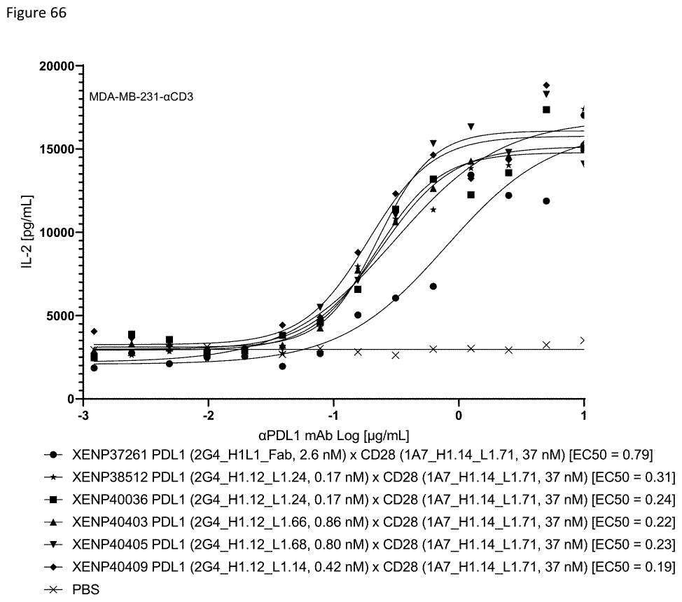

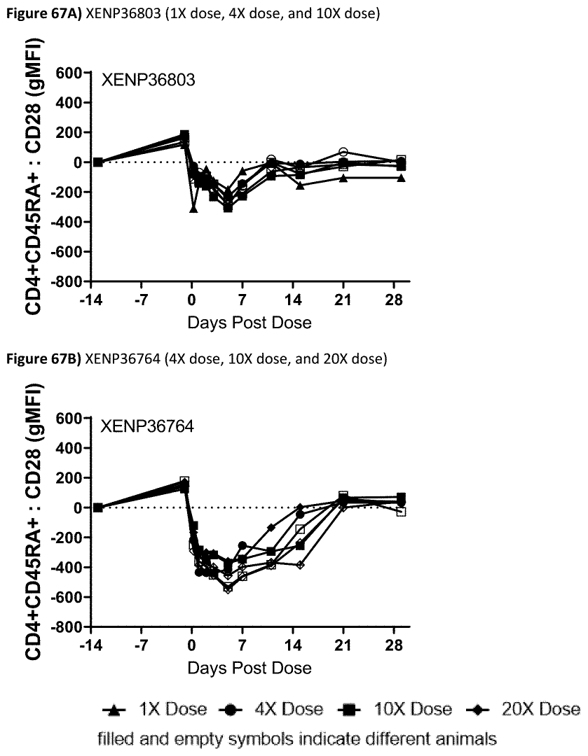

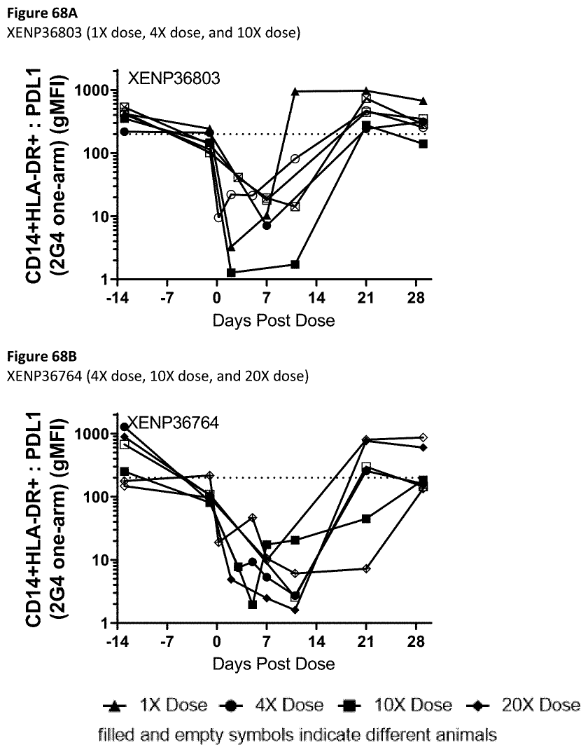

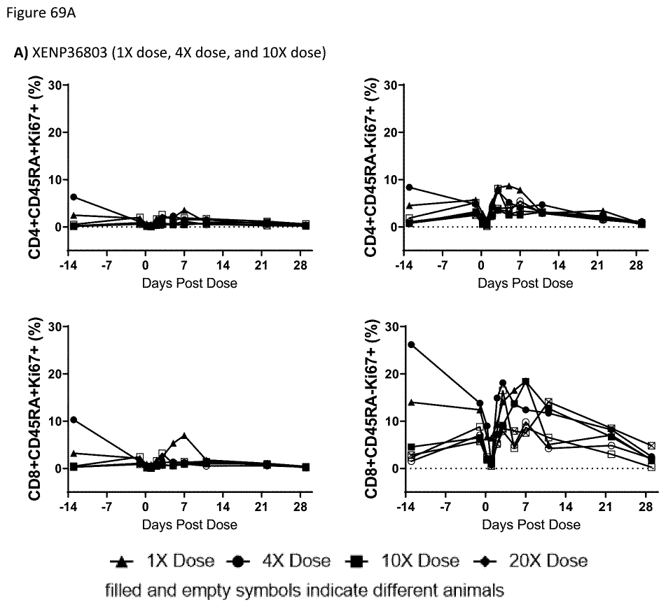

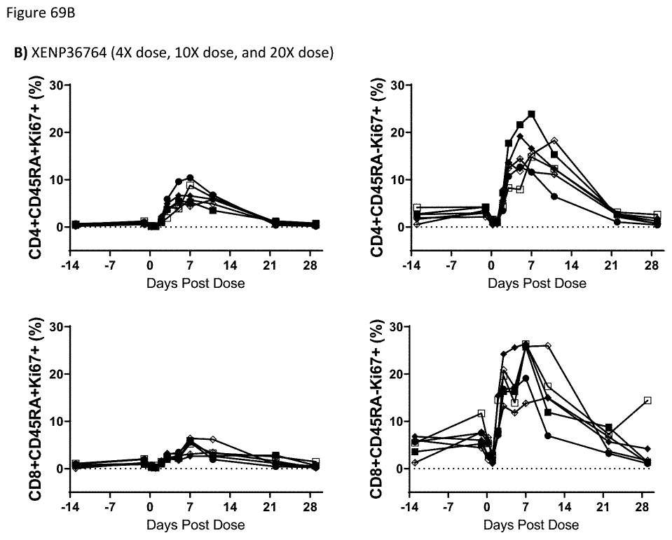

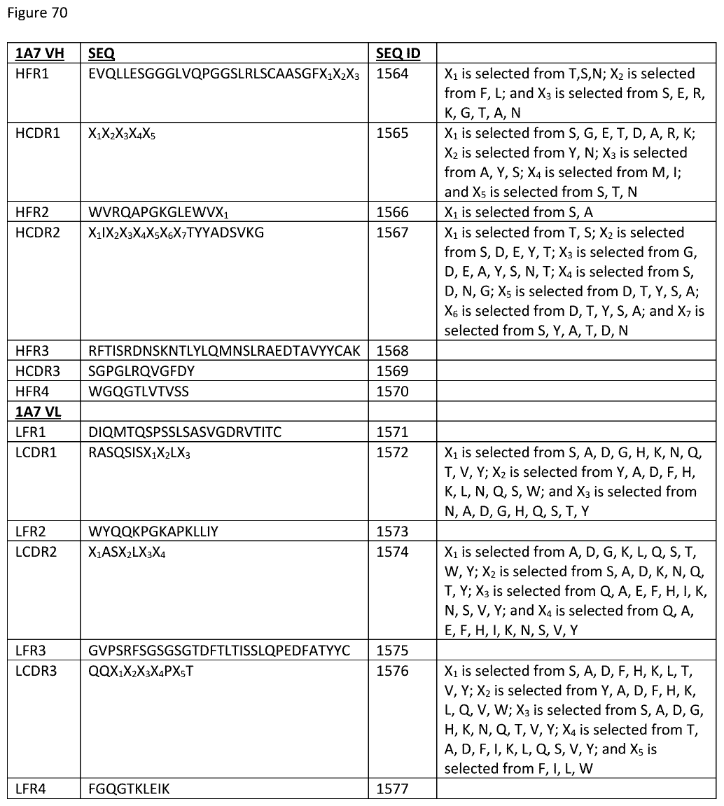

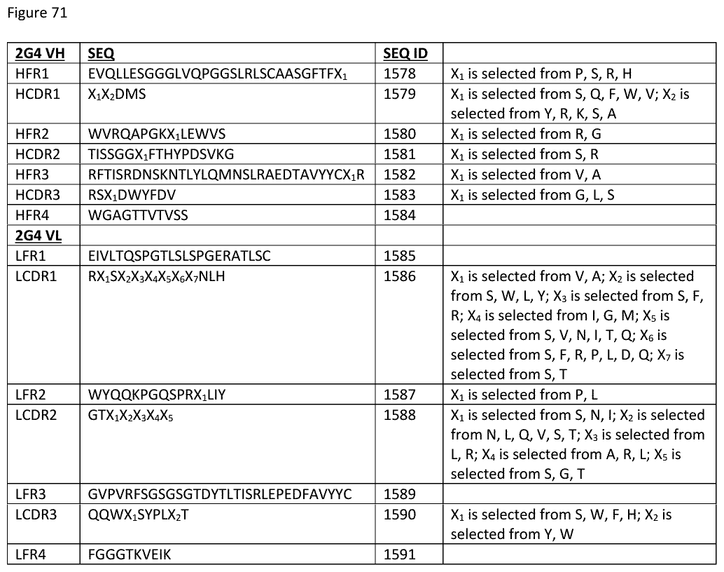

FIG. 12 depicts sequences for “CH1+ hinge” that find use in embodiments of a PD-L1×αCD28 bsAbs that utilize a Fab binding domain. The “CH1+ hinge” sequences find use linking the variable heavy domain (VH) to the Fc backbones (as depicted in FIG. 10). For particular embodiments wherein the Fab is on the (+) side, the “CH1(+)+ hinge” sequences may find use. For particular embodiments wherein the Fab is on the (−) side, the “CH1(−)+ hinge” sequences may find use.

FIG. 13 depicts sequences for “CH1+half hinge” domain linker that find use in embodiments of a PD-L1×αCD28 bsAbs in the 2+1 Fab2-scFv-Fc format or 2+1 CLC format. In the 2+1 Fab2-scFv-Fc format, the “CH1+half hinge” sequences find use linking the variable heavy domain (VH) to the scFv domain on the Fab-scFv-Fc side of the bispecific antibody. In the 2+1 CLC format, the “CH1+half hinge” sequences find use linking the first variable heavy domain (VH) to the second VH domain on the Fab-Fab-Fc side of the bispecific antibody. It should be noted that other linkers may be used in place of the “CH1+half hinge”. It should also be noted that although the sequences here are based on the IgG1 sequence, equivalents can be constructed based on the IgG2 or IgG4 sequences.

FIG. 14 depicts sequences for “CH1” that find use in embodiments of αPDL1×αCD28 bsAbs.

FIG. 15 depicts sequences for “hinge” that find use in embodiments of αPDL1×αCD28 bsAbs.

FIG. 16 depicts the constant domain of the cognate light chains that find use in the subject αPD-L1×αCD28 bsAbs that utilize a Fab binding domain.

FIG. 17 depicts the sequences for XENP16432, an anti-PD-1 mAb based on nivolumab and IgG1 backbone with E233P/L234V/L235A/G236del/S267K ablation variant. CDRs are underlined and slashes indicate the border(s) between the variable regions and constant domain.

FIG. 18 depicts the variable heavy and variable light chain sequences for 1A7, an exemplary phage-derived CD28 binding domain, as well as the sequences for XENP28428, an anti-CD28 mAb based on 1A7 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 19 depicts the sequences for affinity-optimized variable heavy domains from anti-CD28 clone 1A7. It should be noted that the variable heavy domains can be paired with any of the other variable light domains depicted herein including SEQ ID NO: 1002 (e.g. 1A7_H1.1_L1.71 as utilized in XENP37559).

FIG. 20 depicts the sequences for affinity-optimized variable light domain from anti-CD28 clone 1A7. It should be noted that the variable light domains can be paired with any of the other variable heavy domains depicted herein including SEQ ID NOs: 999 and 998 (e.g. 1A7_H1.1_L1.71 as utilized in XENP37559).

FIGS. 21A and 21B depict the sequence for illustrative affinity-optimized 1A7 VH/VH pairs. It should be noted that these pairs may be formatted as Fabs or as scFvs.

FIG. 22 depicts illustrative affinity-engineered 1A7 VH/VL pairs and their binding affinities in the context of scFvs (in the context of 1+1 Fab-scFv-Fc bsAb format).

FIG. 23 depicts the sequences for XENP27181, a bivalent anti-CD28 mAb based on HuTN228 binding domain and IgG1 backbone with E233P/L234V/L235A/G236del/S267K ablation variant; and XENP27656, a monovalent anti-CD28 mAb based on HuTN228 binding domain (formatted as an scFv) and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 24 depicts KDapp (KD apparent due to bivalent binding) of various CD28 binding phage clones (formatted as bivalent mAbs) for human CD28 as determined by Octet. First 60 seconds of dissociation was used for data fit.

FIG. 25 depicts binding of illustrative bivalent anti-CD28 mAbs based on phage-derived clones on human PBMCs. The data show that the phage campaign generated CD28 binding domains having weaker maximum binding than prior art HuTN228 (which is related to the humanized CD28 binding domains described in Example 1A).

FIGS. 26A-26E depicts a couple of formats of the present invention. FIG. 26A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 26B depicts the “2+1 Fab2-scFv-Fc” format, with a first Fab arm and a second Fab-scFv arm, wherein the Fab binds a first antigen and the scFv binds second antigen. The 2+1 Fab2-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 26C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 26D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 26E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen.

FIGS. 27A-27E depicts a couple of formats of the present invention as utilized in CD28 bispecific antibodies. FIG. 27A depicts the “1+1 Fab-scFv-Fc” format, with a first Fab arm binding a tumor-associated antigen and a second scFv arm binding CD28. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 27B depicts the “2+1 Fab₂-scFv-Fc” format, with a first Fab arm and a second Fab-scFv arm, wherein the Fab binds a tumor-associated antigen and the scFv binds CD28. The 2+1 Fab₂-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 27C depicts the “1+1 Common Light Chain” or “1+1 CLC” format, with a first Fc comprising a first Fab arm binding a tumor-associated antigen and a second Fc comprising a second Fab arm binding CD28. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 27D depicts the “2+1 Common Light Chain” or “2+1 CLC” format, with a first Fc comprising 2 Fab arms each binding a tumor-associated antigen and a second Fc comprising 1 Fab arm binding CD28. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 27E depicts the “2+1 mAb-scFv” format, with a first Fc comprising an N-terminal Fab arm binding a tumor-associated antigen and a second Fc comprising an N-terminal Fab arm binding a tumor-associated antigen and a C-terminal scFv binding CD28. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the tumor-associated antigen.

FIG. 28 depicts the variable heavy and variable light chain sequences for 2G4, an exemplary humanized hybridoma-derived PD-L1 binding domain, as well as the sequences for XENP25859, an anti-PD-L1 mAb based on 2G4 and IgG1 backbone with E233P/L234V/L235A/G236del/S267K ablation variant, and XENP36627, a monovalent anti-PD-L1 mAb based on 2G4. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 29A-29C depict the sequences for affinity-optimized variable heavy domains from anti-PDL1 clone 2G4. It should be noted that the variable heavy domains can be paired with any of the other variable light domains depicted herein including SEQ ID NOs: 1046 and 1103-1155 (see FIGS. 28 and 30A-30C, e.g. 2G4_H1.12_L1.14 as utilized in XENP40706).

FIGS. 30A-30C depict the sequences for affinity-optimized variable light domain from anti-PDL1 clone 2G4. It should be noted that the variable light domains can be paired with any of the other variable heavy domains depicted herein including SEQ ID NOs: 1042 and 1055-1099 (see FIGS. 28 and 29A-29C, e.g. 2G4_H1.12_L1.14 as utilized in XENP40706).

FIGS. 31A-31G depict the sequence for illustrative affinity-optimized 2G4 VH/VL pairs. It should be noted that these pairs may be formatted as Fabs or as scFvs.

FIG. 32 depicts A) classic T cell/APC interaction and B) replication of the classic T cell/APC interaction by combining CD3 bispecific antibodies with CD28 bispecific antibodies. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells. In contrast in treatment with CD3 bispecifics, only the first signal is provided. The CD28 signal may be provided by a CD28 bispecific with the idea to promote activation and proliferation through CD28 costimulation.

FIG. 33 depicts the introduction of CD28 signaling by a CD28 bispecific antibody and mitigation of any checkpoint mediated repression of the added CD28 signal by checkpoint blockade (e.g. PD-1 blockade).

FIG. 34 depicts that PD-L1×CD28 bispecific antibodies provide Signal 2 while advantageously further enabling blockade of PD-L1:PD1 interaction.

FIGS. 35A-35S depict the sequences for illustrative αPD-L1×αCD28 bsAbs in the 1+1 Fab-scFv-Fc format. CDRs are underlined, linkers are double-underlined, and slashes indicate the border(s) between the variable regions, Fc regions, and constant domains. It should be noted that the αPD-L1×αCD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

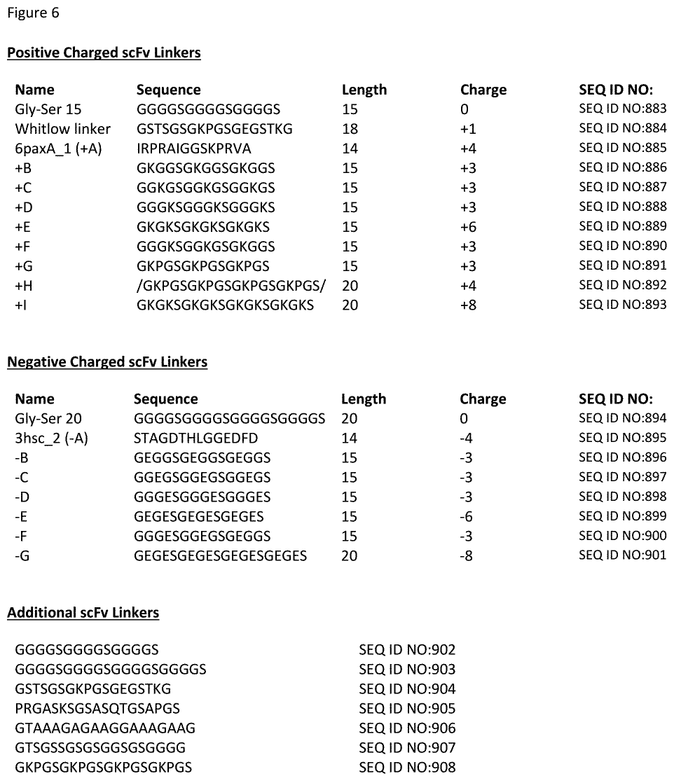

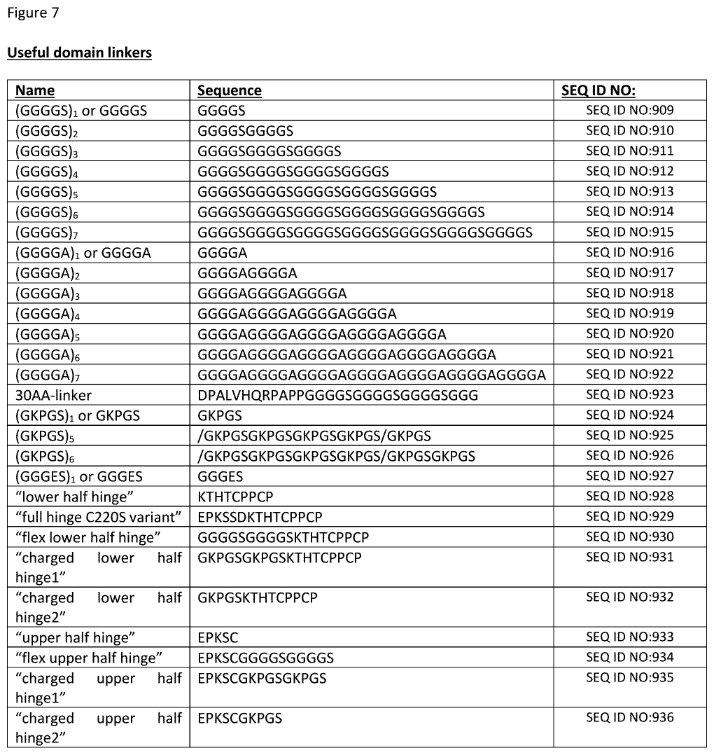

FIG. 36 depicts the sequences for illustrative αPD-L1×αCD28 bsAbs in the 2+1 Fab₂-scFv-Fc format. CDRs are underlined, linkers are double-underlined, and slashes indicate the border(s) between the variable regions, Fc regions, and constant domains. The scFv domain has orientation (N- to C-terminus) of VH-scFv linker-VL, although this can be reversed. It should be noted that the scFv domain sequences includes as the scFv linker between the variable heavy and variable light region the sequence/GKPGSGKPGSGKPGSGKPGS/(SEQ ID NO:892); however, this linker can be replaced with any of the scFv linkers in FIG. 6. It should also be noted that the Chain 2 sequences include as the domain linker between the C-terminus of the scFv and the N-terminus of the CH2 domain the sequence GGGGSGGGGSKTHTCPPCP (SEQ ID NO:930), which is a “flex half hinge” domain linker; however, this linker can be replaced with any of the “useful domain linkers” of FIG. 7. It should be noted that the αPD-L1×αCD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 37 depicts the sequences for illustrative αPDL1×αCD28 bsAbs in the 2+1 mAb-scFv format. CDRs are underlined and slashes indicate the border(s) between the variable regions, linkers, Fc regions, and constant domains. The scFv domain has orientation (N- to C-terminus) of VH-scFv linker-V_{L}, although this can be reversed. It should be noted that the Chain 2 sequences include as a domain linker the sequence/GKPGSGKPGSGKPGSGKPGS/(SEQ ID NO:892); however, this linker can be replaced with any domain linker include any of the “useful domain linkers” of FIG. 6. It should be noted that the αPDL1×αCD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 38 depicts the sequences for XENP24118, an anti-PD-L1 mAb based on avelumab and IgG1 backbone with E233P/L234V/L235A/G236del/S267K ablation variant. CDRs are underlined and slashes indicated border(s) between the variable region and constant domain.

FIG. 39 depicts expansion of CMV+ T cells following incubation of NLV-loaded MDA-MB-231 cancer cells with CD3+ T cells purified from a CMV+ donor and either αPD-L1 antibody XENP24118 or αPD-L1×αCD28 bsAb XENP34963. αPD-L1×αCD28 bsAb XENP34963 significantly enhanced T cell expansion in comparison to PD-L1 blockade alone.

FIG. 40 depicts induction of A) IL-2 secretion, B) IFNγ secretion, and C) CD3+ T cell expansion by 1 μg/ml αB7H3×αCD3 bsAb in combination with αPD-L1 mAb XENP24118 or in combination with αPD-L1×αCD28 bsAb XENP34963. αPD-L1×αCD28 bsAb enhances activity of a CD3 bsAb T cell engager.

FIGS. 41A and 41B depict the sequences for illustrative αPSMA×αCD3 bsAbs in the 2+1 Fab₂-scFv-Fc format and respectively comprising a H1.30_L1.47 anti-CD3 scFv (a.k.a. CD3 High [VHVL]) or a L1.47_H1.32 anti-CD3 scFv (a.k.a. CD3 High-Int #1 [VLVH]). CDRs are underlined and slashes indicate the border(s) between the variable regions and other chain components (e.g. constant region and domain linkers). It should be noted that the αPSMA×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 42 depicts cell kill over time following incubation of LNCaP cancer cells (PSMA+) with CD3+ T cells at a 1:1 effector:target ratio and illustrative CD3 bispecific (αPSMA×αCD3 XENP32220) alone or in combination with XENP36233. The data show that XENP32220 enhanced cell kill in comparison to incubation of cancer and T cells alone; however, addition of αPD-L1×αCD28 overcomes cancer cell resistance to the CD3 bispecific and further enhances cell kill.

FIGS. 43A-43B depicts group median change in tumor volume (as determined by caliper measurement; baseline corrected) A) over time (in days after first dose) and B) Day 20 (after first dose) in MC38 (engineered to stably expressing human PD-L1) and huPBMC-engrafted human CD28 knock-in mice dosed with 5 mg/kg αPD-L1 mAb XENP24118, 8.3 mg/kg αPD-L1×αCD28 bsAb XENP34963, 6 mg/kg αPD-L1×αCD28 XENP34961, or PBS control.

FIGS. 44A-44L depict sequences for exemplary anti-CD3 binding domains suitable for use in CD3 bispecific antibodies which may be combined with the CD28 bispecific antibodies of the invention. The CDRs are underlined, the scFv linker is double underlined (in the sequences, the scFv linker is a positively charged scFv (GKPGS)₄ linker (SEQ ID NO:892), although as will be appreciated by those in the art, this linker can be replaced by other linkers, including uncharged or negatively charged linkers, some of which are depicted in FIG. 6), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 45 depicts the sequence for XENP29154 a.k.a. TGN1412.

FIG. 46 depicts the release of IFNγ from human PBMCs treated with air-dried XENP28428 (anti-CD28 clone 1A7), TGN1412 (XENP29154), or negative control PBS.

FIG. 47 depicts the dissociation constant (K_{D}— as an average of result from HIS1K capture vs. streptavidin capture) and corresponding sensorgrams (either HIS1K capture or streptavidin capture) of αPDL1×αCD28 bsAbs having affinity-engineered CD28 binding domains.

FIG. 48 depicts PD1:PDL1 blockade (binding of PDL1-mFc fusion to PD1-transfected HEK293T cells) by anti-PDL1 clone 2G4 (XENP25859), a partial blocking anti-PDL1 (XENP25853), a non-blocking anti-PDL1 (XENP25858), and XENP24118 (a benchmark anti-PDL1 mAb based on avelumab).

FIG. 49 depicts induction of A) IL-2 and B) IFNγ release by anti-PDL1 clone 2G4 (XENP25859), a partial blocking anti-PDL1 (XENP25853), a non-blocking anti-PDL1 (XENP25858), and XENP24118 (a benchmark anti-PDL1 mAb based on avelumab). The data show that the partial blocking and non-blocking anti-PDL1 clones induced less cytokine release in comparison to anti-PDL1 clone 2G4.

FIG. 50 depicts the dissociation constant (K_{D}) of αPDL1×αCD28 bsAbs (1+1 Fab-scFv-Fc format) having affinity-engineered PDL1 binding domains (as well as K_{D} fold improvement over αPDL1×αCD28 bsAb having WT 2G4 binding domain).

FIG. 51 depicts blockade of PD1:PDL1 interaction during T cell:cancer cell interaction (as modeled by Jurkat-PD1 cells incubated with CHO-PDL1-CD80-αCD3 and CHO-PDL1-αCD3 cells) by αPDL1×αCD28 bsAbs having anti-PDL1 clone 2G4 (XENP36233), a partial blocking anti-PDL1 (XENP36232), a non-blocking anti-PDL1 (XENP26783), and XENP34963 (a benchmark bsAb with an anti-PDL1 arm based on avelumab). The data show that bsAbs comprising partial blocking and non-blocking anti-PDL1 clones induced less activity in comparison to bsAb comprising anti-PDL1 clone 2G4.

FIG. 52 depicts binding of αPDL1×αCD28 (XENP36233) to parental PDL1^{null} MC38 or MC38 cells transfected to express PDL1 with low or medium high antigen densities.

FIG. 53 depicts induction of IL-2 release by αPDL1×αCD28 (XENP36233) in the presence of parental PDL1^{null} HEK293T cells or HEK293T transfected to express PDL1 with medium or high antigen densities.

FIG. 54 depicts induction of cell kill by αPSMA×αCD3 alone or in combination with αPDL1×αCD28 XENP36233 in the presence of CD3+ T cells and PDL1^{null} 22Rv1 cell line at A) 10:1 E:T ratio and B) 1:1 E:T ratio. The data show that αPDL1×αCD28 bsAbs do not synergize with CD3 bsAbs on PDL1 negative cell lines such as 22Rv1.

FIG. 55 depicts serum concentration of XENP36764 over time in cynomolgus monkeys. The αPDL1×αCD28 bsAb exhibited favorable pharmacokinetics.

FIG. 56 depicts diagram of assumptions used in a mechanism-based PK/PD computer model.

FIGS. 57A-57C depicts predictions from the mechanism-based modeling suggesting A) linear PK at dose levels consistent with typical checkpoint inhibitor regimens, B) trimer formation in the tumor indicating costimulation, and C) consistent blockade of PDL1.

FIG. 58 depicts induction of IL-2 release by αPDL1×αCD28 bsAbs having affinity-engineered CD28 binding domains in the presence of αB7H3×αCD3 bsAb, CD3+T cells, and A) MDA-MB-231 or B) DU145-NLR cells. The data show that increasing CD28 affinity leads to more potent and efficacious IL-2 secretion by αPDL1×αCD28 bsAbs.

FIG. 59 depicts induction of cell kill by αPDL1×αCD28 bsAbs having affinity-engineered CD28 binding domains in the presence of αB7H3×αCD3 bsAb, CD3+ T cells, and PDL1^{low} LnCAP cancer cells. The data show that increasing the affinity of CD28 increases targeting of PDL1^{low} cancer cells at low E:T of 1:1.

FIG. 60 depicts induction of cell kill by αPDL1×αCD28 bsAbs having affinity-engineered CD28 binding domains in the presence of αB7H3×αCD3 bsAb, CD3+ T cells, and PDL1^{med} DU145 cells. The data show that increasing the affinity of CD28 increases targeting of PDL1^{med} cancer cells at even lower E:T of 0.1:1.

FIGS. 61A-61D depict change in tumor volume (as determined by caliper measurements; baseline corrected) in individual mouse over time and D) on Day 28 in hPDL1-MC38-engrafted hCD28 knock-in mice dosed with A) PBS control, B) monovalent αPDL1 mAb XENP36627, and C) XENP37261 having enhanced CD28 binding affinity.

FIG. 62 depicts induction of IL-2 release by αPDL1×αCD28 bsAbs having affinity-engineered PDL1 binding domains in the presence of αB7H3×αCD3 bsAb, CD3+ T cells, and DU145-NLR cells. The data show that increasing PDL1 affinity promotes IL-2 secretion.

FIGS. 63A and 63B depict induction of A) IL2 and B) IFNγ release by αPDL1 mAb XENP24118 and αPDL1×αCD28 bsAb XENP38514 having enhanced PDL1 binding in a DC: T cell MLR. The data show that αPDL1×αCD28 enhanced T cell/APC interaction.

FIG. 64 depicts PD1:PDL1 blockade (binding of PDL1-mFc fusion to PD1-transfected HEK293T cells) by αPDL1×αCD28 bsAbs having affinity-engineered PDL1 binding domains. The data show the αPDL1×αCD28 bsAbs can block interaction between PD1 and PDL1.

FIG. 65 depicts induction of IL-2 release by αPDL1×αCD28 bsAbs having affinity-engineered CD28 binding domains and affinity-engineered PDL1 binding domains in the presence of SEB-stimulated PBMCs. The data show that increasing PDL1 affinity promotes IL-2 secretion.

FIG. 66 depicts induction of IL-2 release by αPDL1×αCD28 bsAbs having affinity-engineered CD28 binding domains and affinity-engineered PDL1 binding domains in the presence of CD3+ enriched T cells, MDA-MB0231 transfected to express αCD3 scFv (to act as Signal 1), and 1 μg/mL of an illustrative B7H3×CD3 bsAb. The data show that XENP40409 (non-Xtend analog of XENP40706) having 2G4_H1.12_L1.14 induced IL2 production most potently.

FIGS. 67A and 67B depict CD28 receptor occupancy on cynomolgus T cells (as indicated by decrease in binding by secondary CD28 mAb) after dosing with A) XENP36803 (1× dose, 4× dose, and 10× dose) or B) XENP36764 (4× dose, 10× dose, and 20× dose). The data show CD28 receptor occupancy up to day 14 on T cells.

FIGS. 68A and 68B depict PDL1 receptor occupancy on cynomolgus activated monocytes (decrease in free receptor as indicated by decrease in binding by one-arm PDL1 mAb based on 2G4) after dosing with A) XENP36803 (1× dose, 4× dose, and 10× dose) or B) XENP36764 (4× dose, 10× dose, and 20× dose).

FIGS. 69A and 69B depict proliferation of cynomolgus T cells (as indicated by increased Ki67 expression) after dosing with A) XENP36803 (1× dose, 4× dose, and 10× dose) or B) XENP36764 (4× dose, 10× dose, and 20× dose). Notably, the PDL1×CD28 bsAbs selectively induce proliferation of effector CD4+ and CD8+ T cells (i.e. CD45RA−).

FIG. 70 depicts consensus framework regions (FR) and complementarity determining regions (CDRs) (as in Kabat) for anti-CD28 clone 1A7 variable heavy and variable light domain variants. In some embodiments, the CD28 antigen binding domain provided herein includes one or more of the sequences depicted in FIG. 70.

FIG. 71 depicts consensus framework regions (FR) and complementarity determining regions (CDRs) (as in Kabat) for anti-PDL1 clone 2G4 variable heavy and variable light domain variants. In some embodiments, the PD-L1 antigen binding domain provided herein includes one or more of the sequences depicted in FIG. 71.

### DETAILED DESCRIPTION

### I. Overview

Provided herein are novel anti-PD-L1 antibodies, including novel anti-CD28×anti-PD-L1 (also referred to as “αCD28×αPD-L1”) bispecific antibodies, and methods of using such antibodies for the treatment of cancers. Subject αCD28×αPD-L1 antibodies are capable of agonistically binding to CD28 costimulatory molecules on T cells and targeting to PD-L1 on tumor cells. Thus, such antibodies selectively enhance anti-tumor activity at tumor sites while minimizing peripheral toxicity. The subject antibodies provided herein are particularly useful for enhancing anti-tumor activity when used in combination with other anti-cancer therapies.

Accordingly, in one aspect, provided herein are heterodimeric antibodies that bind to two different antigens, e.g., the antibodies are “bispecific,” in that they bind two different target antigens, generally CD28 and PD-L1 as described below. These heterodimeric antibodies can bind each of the target antigens either monovalently (e.g., there is a single antigen binding domain such as a variable heavy and variable light domain pair) or bivalently (there are two antigen binding domains that each independently bind the antigen). In some embodiments, the heterodimeric antibody provided herein includes one CD28 binding domain and one PD-L1 binding domain (e.g., heterodimeric antibodies in the “1+1 Fab-scFv-Fc” format described herein). In other embodiments, the heterodimeric antibody provided herein includes one CD28 binding domain and two PD-L1 binding domains (e.g., heterodimeric antibodies in the “2+1 Fab₂-scFv-Fc” formats described herein). The heterodimeric antibodies provided herein are based on the use of different monomers that contain amino acid substitutions (i.e., skew variants”) that “skew” formation of heterodimers over homodimers, as is more fully outlined below. In some embodiments, the heterodimer antibodies are also coupled with “pI variants” that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

The antibodies provided herein are listed in several different formats. In some instances, each monomer of a particular antibody is given a unique “XENP” number, although as will be appreciated in the art, a longer sequence might contain a shorter one. For example, a “scFv-Fc” monomer of a 1+1 Fab-scFv-Fc format antibody may have a first XENP number, while the scFv domain itself will have a different XENP number. Some molecules have three polypeptides, so the XENP number, with the components, is used as a name. Thus, the molecule XENP36154, which is in 2+1 Fab₂-scFv-Fc format, comprises three sequences (see FIG. 36) a “Fab-Fc Heavy Chain” monomer (“Chain 1”); 2) a “Fab-scFv-Fc Heavy Chain” monomer (“Chain 2”); and 3) a “Light Chain” monomer or equivalents, although one of skill in the art would be able to identify these easily through sequence alignment. These XENP numbers are in the sequence listing as well as identifiers, and used in the Figures. In addition, one molecule, comprising the three components, gives rise to multiple sequence identifiers. For example, the listing of the Fab includes the full heavy chain sequence, the variable heavy domain sequence and the three CDRs of the variable heavy domain sequence, the full light chain sequence, a variable light domain sequence and the three CDRs of the variable light domain sequence. A Fab-scFv-Fc monomer includes a full-length sequence, a variable heavy domain sequence, 3 heavy CDR sequences, and an scFv sequence (include scFv variable heavy domain sequence, scFv variable light domain sequence and scFv linker). Note that some molecules herein with a scFv domain use a single charged scFv linker (+H), although others can be used. In addition, the naming nomenclature of particular antigen binding domains (e.g., PD-L1 and CD28 binding domains) use a “Hx.xx_Ly.yy” or “Hx.xxLy.yy” type of format, with the numbers being unique identifiers to particular variable chain sequences. Thus, the variable domain of the Fab side of PD-L1 binding domain 2G4[PDL1](e.g., FIG. 28) is “H1L1”, which indicates that the variable heavy domain, H1, was combined with the light domain L1. In the case that these sequences are used as scFvs, the designation “H1_L1” or “H1L1”, indicates that the variable heavy domain, H1 is combined with the light domain, L1, and is in VH-linker-VL orientation, from N- to C-terminus. This molecule with the identical sequences of the heavy and light variable domains but in the reverse order (VL-linker-VH orientation, from N- to C-terminus) would be designated “L1_H”. Similarly, different constructs may “mix and match” the heavy and light chains as will be evident from the sequence listing and the figures.

### III. Definitions

In order that the application may be more completely understood, several definitions are set forth below. Such definitions are meant to encompass grammatical equivalents.

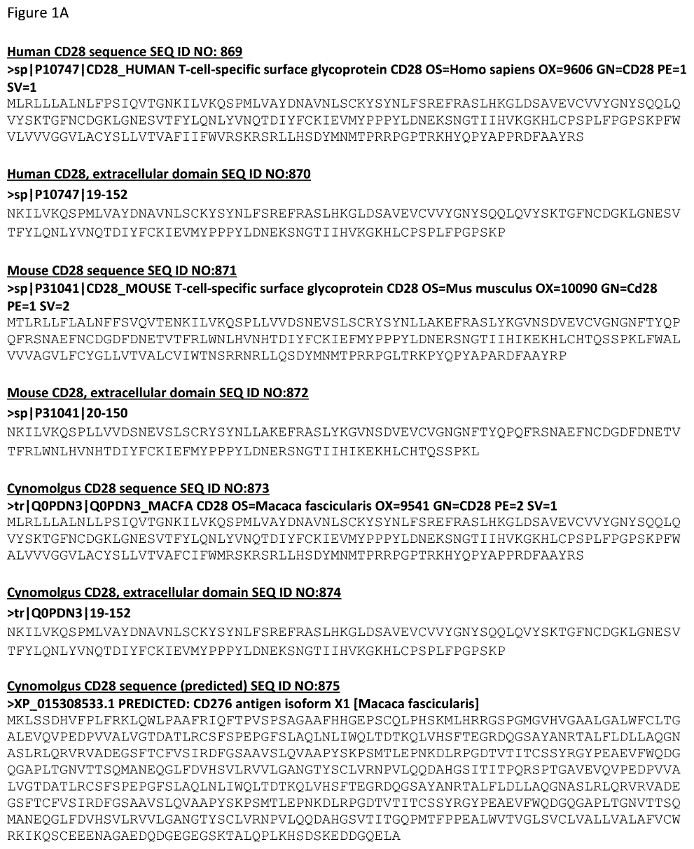

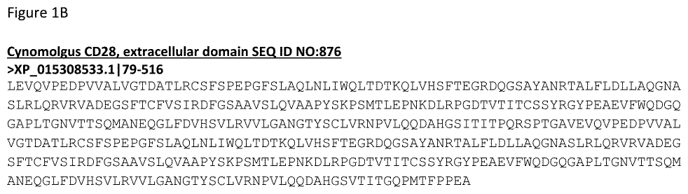

By “CD28,” “Cluster of Differentiation 28,” and “Tp44” (e.g., Genebank Accession Numbers NP_001230006 (human), NP_001230007 (human), NP_006130 (human), and NP_031668 (mouse)) herein is meant a B7 receptor expressed on T cells that provides co-stimulatory signals required for T cell activation and survival. T cell stimulation through CD28 in addition to the T cell receptor (TCR) provides a potent signal for the production of various interleukins. CD28 is the receptor for CD80 (B7.1) and CD86 (B7.2) proteins. CD28 includes an intercellular domain with a YMNM motif critical for the recruitment of SH2-domain containing proteins, particularly PI3K. CD28 also includes two proline-rich motifs that are able to bind SH3-containing proteins. Exemplary CD28 sequences are depicted in FIG. 1.

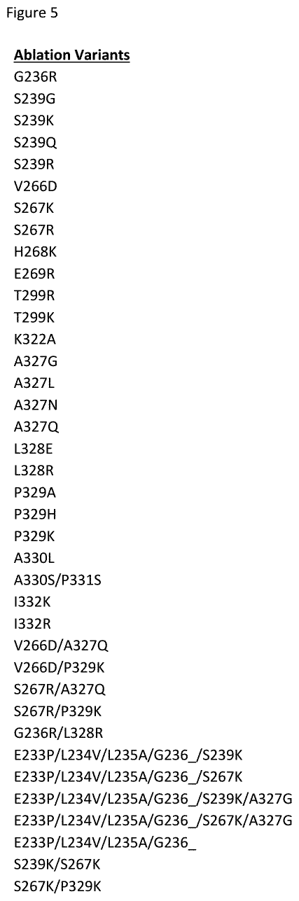

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 5, which generally are added to both monomers.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

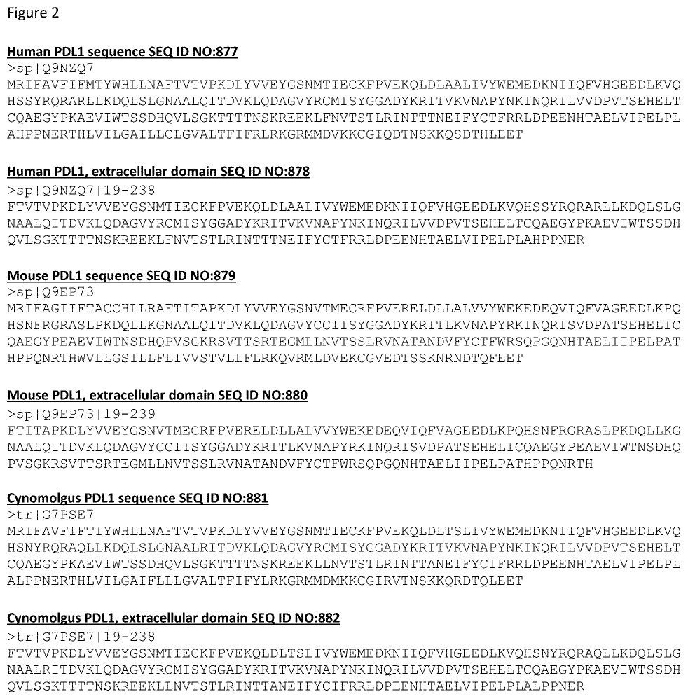

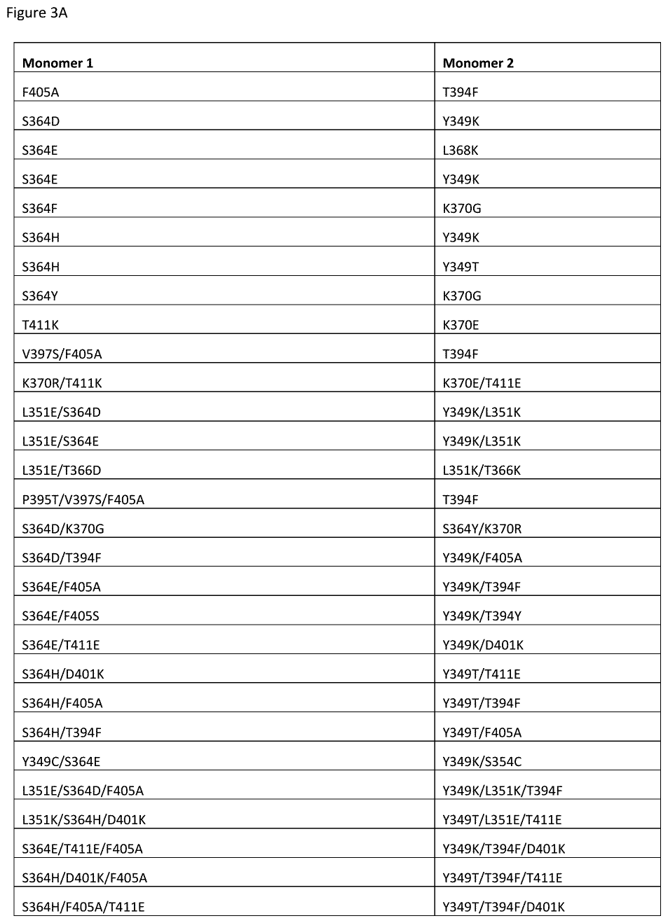

Other useful antibody formats include, but are not limited to, the “1+1 Fab-scFv-Fc,” “2+1 Fab₂-scFv-Fc,” “1+1 common light chain,” and “2+1 common light chain” formats provided herein (see, e.g., FIG. 26). Additional useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in US20180127501A1, which is incorporated by reference herein, particularly in pertinent part relating to antibody formats (see, e.g., FIG. 2).

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., PD-L1 or CD28) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used (e.g., from FIG. 26). In general, the C-terminus of the scFv domain is attached to the N-terminus of the hinge in the second monomer.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, v1CDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233#, E233( ) or E233del designates a deletion of glutamic acid at position 233. Additionally, EDA233- or EDA233# designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fe domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### IV. PD-L1 and CD28 Antigen Binding Domains

Provided herein are antigen binding domains (ABDs) and ABD compositions that bind either PD-L1 or CD28. In some embodiments, one or more of the ABDs are included in an antibody format described herein including, for example, “1+1 Fab-scFv-Fc,” “2+1 Fab₂-scFv-Fc,” “2+1 mAb-scFv,” “1+1 common light chain,” and “2+1 common light chain” antibodies.

### A. PD-L1 Antigen Binding Domains

In one aspect, provided herein are PD-L1 antigen binding domains (ABDs) and compositions that include such PD-L1 antigen binding domains (ABDs), including anti-PD-L1 antibodies. Such PD-L1 binding domains and related antibodies (e.g., anti-PD-L1×anti-CD28 bispecific antibodies) find use, for example, in the treatment of PD-L1 associated cancers. In some embodiments, the PD-L1 ABDs are capable of binding to human and cynomolgus PD-L1 (see FIG. 2 and Example 2).

As will be appreciated by those in the art, suitable PD-L1 binding domains can comprise a set of 6 CDRs as depicted in FIGS. 28-31 and the Sequence Listing. Suitable PD-L1 ABDs can also include the entire VH and VL sequences as depicted in these sequences and FIGS. 28-31 and the Sequence Listing, used as scFvs or as Fab domains.

In one embodiment, the PD-L1 antigen binding domain includes the 6 CDRs (i.e., vhCDR1-3 and vlCDR1-3) of a PD-L1 ABD described herein, including FIGS. 28-31 and the Sequence Listing, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 28-31 and the Sequence Listing (see Table 2). Suitable PD-L1 ABDs can also include the entire VH and VL sequences as depicted in these sequences and figures, used as scFvs or as Fab domains. Additional anti-PD-L1 ABDs are shown in FIGS. 15, 73 and 78 of U.S. Pat. No. 10,793,632, as well as SEQ ID Nos:3961 to 4432 of that application, all of which sequences are incorporated herein by reference.

In one embodiment, the PD-L1 antigen binding domain includes the 6 CDRs (i.e., vhCDR1-3 and vlCDR1-3) of an PD-L1 ABD described herein, including FIGS. 28-31 and the Sequence Listing. In exemplary embodiments, the PD-L1 ABD is one of the following PD-L1 ABDs: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L0.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29, 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SHID1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, and PDL1.3.1 (FIGS. 28-31 and the Sequence Listing).

In addition to the parental CDR sets disclosed in the figures and sequence listing that form an ABD to PD-L1, provided herein are variant PD-L1 ABDS having CDRs that include at least one modification of the PD-L1 ABD CDRs disclosed herein. In one embodiment, the PD-L1 ABD includes a set of 6 CDRs with 1, 2, 3, 4, 5, 6, 7, 8, 9, 10 amino acid modifications as compared to the 6 CDRs of an PD-L1 ABD described herein, including the figures and sequence listing. In exemplary embodiments, the PD-L1 ABD includes a set of 6 CDRs with 1, 2, 3, 4, 5, 6, 7, 8, 9, 10 amino acid modifications as compared to the 6 CDRs of one of the following 2G4 ABDs: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29, 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var1, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SHID1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, and PDL1.3.1 (FIGS. 28-31 and the Sequence Listing). In certain embodiments, the variant PD-L1 ABD is capable of binding PD-L1 antigen, as measured by at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In one embodiment, the anti-PD-L1 ABD includes 6 CDRs that are at least 90, 95, 97, 98 or 99% identical to the 6 CDRs of an anti-PD-L1 ABD as described herein, including FIGS. 28-31 and the Sequence Listing. In exemplary embodiments, the anti-PD-L1 ABD includes 6 CDRs that are at least 90, 95, 97, 98 or 99% identical to the 6 CDRs of one of the following PD-L1 ABDs: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29, 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var1, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SHID1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, and PDL1.3.1 (FIGS. 28-31 and the Sequence Listing). In certain embodiments, the anti-PD-L1 ABD is capable of binding to PD-L1 antigen, as measured by at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In another exemplary embodiment, the anti-PD-L1 ABD include the variable heavy (VH) domain and/or variable light (VL) domain of any one of the PD-L1 ABDs described herein, including FIGS. 28-31 and the Sequence Listing. In exemplary embodiments, the anti-PD-L1 ABD is 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L0.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29, 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SH1D1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, and PDL1.3.1 (FIGS. 28-31 and the Sequence Listing).

In some embodiments, the PD-L1 ABD includes one of the following PD-L1 ABD variable heavy domains: SEQ ID NO: 1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, and SEQ ID NO:1099, SEQ ID NOs:736-797.

In some embodiments, the PD-L1 ABD includes one of the following PD-L1 ABD variable light domains: SEQ ID NO:1111, SEQ ID NO:1046, SEQ ID NO: 1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868.

Any of the PD-L1 ABD variable heavy domain or variants thereof disclosed herein can be combined with any of the variable light domains or variants thereof described herein to form PD-L1 ABDs for use in any of the antibody formats provided herein.

In addition to the parental anti-PD-L1 binding domain variable heavy and variable light domains disclosed herein, provided herein are anti-PD-L1 ABDs that include a variable heavy domain and/or a variable light domain that are variants of an anti-PD-L1 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of an anti-PD-L1 ABD described herein, including FIGS. 28-31 and the Sequence Listing. In exemplary embodiments, the variant VH domain and/or VL domain has from 1, 2, 3, 4, 5, 6, 7, 8, 9 or 10 amino acid changes from a VH and/or VL domain of one of the following 2G4 ABDs: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L0.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29, 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SHID1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, and PDL1.3.1 (FIGS. 28-31 and the Sequence Listing).

In certain embodiments, the anti-PD-L1 ABD is capable of binding to PD-L1, as measured at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In some embodiments, the PD-L1 ABD includes a variable heavy domain that has from 1, 2, 3, 4, 5, 6, 7, 8, 9 or 10 amino acid changes one of the following PD-L1 ABD variable heavy domains: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, and SEQ ID NO:1099, SEQ ID NOs:736-797. In some embodiments, the PD-L1 ABD includes any of the variable light domains provided herein or a variant thereof.

In some embodiments, the PD-L1 ABD includes a variable light domain that has from 1, 2, 3, 4, 5, 6, 7, 8, 9 or 10 amino acid changes one of the following PD-L1 ABD variable light domains: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868. In some embodiments, the PD-L1 ABD includes any of the variable heavy domains provided herein or a variant thereof.

In one embodiment, the variant VH and/or VL domain is at least 90, 95, 97, 98 or 99% identical to the VH and/or VL of an anti-PD-L1 ABD as described herein, including FIGS. 28-31 and the Sequence Listing. In exemplary embodiments, the variant VH and/or VL domain is at least 90, 95, 97, 98 or 99% identical to the VH and/or VL of one of the following 2G4 ABDs: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L0.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29, 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SHID1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, and PDL1.3.1 (FIGS. 28-31 and the Sequence Listing).

In certain embodiments, the anti-PD-L1 ABD is capable of binding to PD-L1, as measured by at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In some embodiments, the PD-L1 ABD includes a variable heavy domain that is at least 90, 95, 97, 98 or 99% identical to one of the following PD-L1 ABD variable heavy domains: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, and SEQ ID NO:1099, SEQ ID NOs:736-797. In some embodiments, the PD-L1 ABD includes any of the variable light domains provided herein or a variant thereof.

In some embodiments, the PD-L1 ABD includes a variable light domain that is at least 90, 95, 97, 98 or 99% identical to one of the following PD-L1 ABD variable light domains: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868. In some embodiments, the PD-L1 ABD includes any of the variable heavy domains provided herein or a variant thereof.

In some embodiments, the PD-L1 ABDs have variable heavy and variable light domains that are 90, 95, 97, 98 or 99% identical to the VH and/or VL domains of a PD-L1 ABD as described herein, but the CDRs are identical. In some embodiments, the VH selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, and SEQ ID NO:1099, SEQ ID NOs:736-797. In some embodiments, the VL is selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868.

In some embodiments, the PD-L1 ABDs have variable heavy and variable light domains that are 90, 95, 97, 98 or 99% identical to the VH and/or VL domains of a PD-L1 ABD as described herein, and there can be from 0 to 6 amino acid modifications in the CDRs. In some embodiments, the VH selected from the group consisting of: SEQ ID NO:1079, SEQ ID NO:1042, SEQ ID NO:1055, SEQ ID NO:1059, SEQ ID NO: 1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, and SEQ ID NO:1099, SEQ ID NOs:736-797. In some embodiments, the VL is selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868.

In some embodiments, the PD-L1 ABDs have variable heavy and variable light domains that are 90, 95, 97, 98 or 99% identical to the VH and/or VL domains of a PD-L1 ABD as described herein, and there can be from 0 to 6 amino acid modifications in the CDRs, with no CDR having more than 1 amino acid modification. In some embodiments, the VH selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, and SEQ ID NO:1099, SEQ ID NOs:736-797. In some embodiments, the VL is selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868.

In some embodiments, the PD-L1 antigen binding domain includes a variable heavy domain (VH) and a variable light domain (VL) selected from the group consisting of: a VH having an amino acid sequence of SEQ ID NO:1183 and a VL having an amino acid sequence of SEQ ID NO:1187; a VH having an amino acid sequence of SEQ ID NO:1042 and a VL having an amino acid sequence of SEQ ID NO:1047; a VH having an amino acid sequence of SEQ ID NO:1159 and a VL having an amino acid sequence of SEQ ID NO:1163; a VH having an amino acid sequence of SEQ ID NO:1167 and a VL having an amino acid sequence of SEQ ID NO:1171; a VH having an amino acid sequence of SEQ ID NO:1175 and a VL having an amino acid sequence of SEQ ID NO:1179; a VH having an amino acid sequence of SEQ ID NO:1191 and a VL having an amino acid sequence of SEQ ID NO:1195; a VH having an amino acid sequence of SEQ ID NO:1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

In certain embodiments, the PD-L1 ABD is capable of binding to the PD-L1, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human PD-L1 (see FIG. 2) at detectable limits of the assay.

In some embodiments, the PD-L1 antigen binding domain provided herein includes one or more of the sequences depicted in FIG. 71. In some embodiments, the PD-L1 antigen binding domain includes at least one of the HCDR1-3 and/or LCDR1-3 sequences of FIG. 71. In some embodiments, the PD-L1 antigen binding domain includes at least one of the HFR1-4 and/or LFR1-4 sequences of FIG. 71.

Such PD-L1 binding domains can be included in any of the antibodies provided herein including, for example, “1+1 Fab-scFv-Fc,” “2+1 Fab₂-scFv-Fc,” “2+1 mAb-scFv,” “1+1 common light chain,” and “2+1 common light chain” antibodies.

### B. CD28 Antigen Binding Domains

In one aspect, provided herein are CD28 antigen binding domains (ABDs) and compositions that include such CD28 antigen binding domains (e.g., antibodies, including the heterodimeric antibodies provided herein). In some embodiments, the CD28 antigen binding domain described herein are agonistic CD28 ABDs that advantageously provide costimulatory activity. Thus, such CD28 ABDs provided herein are useful of enhancing immune responses, for example, when used in combination with other therapeutics (e.g., anti-cancer therapeutics for the treatment of particular cancers).

As will be appreciated by those in the art, suitable CD28 binding domains can comprise a set of 6 CDRs as depicted in the Sequence Listing and Figures, either as they are underlined or, in the case where a different numbering scheme is used as described herein and as shown in Table 2, as the CDRs that are identified using other alignments within the variable heavy (VH) domain and variable light domain (VL) sequences of those depicted in FIGS. 18-23 and the Sequence Listing. Suitable CD28 ABDs can also include the entire VH and VL sequences as depicted in these sequences and figures, used as scFvs or as Fabs.

In one embodiment, the CD28 antigen binding domain includes the 6 CDRs (i.e., vhCDR1-3 and vlCDR1-3) of any of the CD28 binding domains described herein, including the Figures and Sequence Listing. In some embodiments, the CD28 ABD is one of the following CD28 ABDs:

In exemplary embodiments, the CD28 ABD is one of the following CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1 (FIGS. 18-23 and the Sequence Listing).

In addition to the parental CDR sets disclosed in the figures and sequence listing that form an ABD to CD28, provided herein are variant CD28 ABDS having CDRs that include at least one modification of the CD28 ABD CDRs disclosed herein (e.g., FIGS. 18-23 or the Sequence Listing). In one embodiment, the CD28 ABD includes a set of 6 CDRs with 1, 2, 3, 4, 5, 6, 7, 8, 9, 10 amino acid modifications as compared to the 6 CDRs of a CD28 ABD as described herein, including the figures and sequence listing. In exemplary embodiments, the CD28 ABD includes a set of 6 CDRs with 1, 2, 3, 4, 5, 6, 7, 8, 9, 10 amino acid modifications as compared to the 6 CDRs of one of the following CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing).

In certain embodiments, the CD28 ABD is capable of binding CD28 antigen, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 ABD includes variable heavy and variable light domains that are at least 90, 95, 97, 98 or 99% identical to the VH and VL domains of a CD28 ABD as described herein, including the figures and sequence listing. In exemplary embodiments, the CD28 ABD includes VH and VL domains that are at least 90, 95, 97, 98 or 99% identical to the 6 CDRs of one of the following CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing). In some embodiments, the VH selected from the group consisting of: SEQ ID NO:998, SEQ ID NO:984, SEQ ID NO:994 and SEQ ID NOs:561-629. In some embodiments, the VL is selected from the group consisting of: SEQ ID NO:1002, SEQ ID NO:984, and SEQ ID Nos:630-735 or a variant thereof.

In some embodiments, the CD28 ABDs have variable heavy and variable light domains that are 90, 95, 97, 98 or 99% identical to the VH and/or VL domains of a CD28 ABD as described herein, but the CDRs are identical. In some embodiments, the VH selected from the group consisting of: SEQ ID NO:998, SEQ ID NO:984, SEQ ID NO:994 and SEQ ID NOs:561-629. In some embodiments, the VL is selected from the group consisting of: SEQ ID NO:1002, SEQ ID NO:984, and SEQ ID Nos:630-735 or a variant thereof.

In some embodiments, the CD28 ABDs have variable heavy and variable light domains that are 90, 95, 97, 98 or 99% identical to the VH and/or VL domains of a CD28 ABD as described herein, and there can be from 0 to 6 amino acid modifications in the CDRs. In some embodiments, the VH selected from the group consisting of: SEQ ID NO:998, SEQ ID NO:984, SEQ ID NO:994 and SEQ ID NOs:561-629. In some embodiments, the VL is selected from the group consisting of: SEQ ID NO: 1002, SEQ ID NO:984, and SEQ ID Nos:630-735 or a variant thereof.

In some embodiments, the CD28 ABDs have variable heavy and variable light domains that are 90, 95, 97, 98 or 99% identical to the VH and/or VL domains of a CD28 ABD as described herein, and there can be from 0 to 6 amino acid modifications in the CDRs, with no CDR having more than 1 amino acid modification. In some embodiments, the VH selected from the group consisting of: SEQ ID NO:998, SEQ ID NO:984, SEQ ID NO:994 and SEQ ID NOs:561-629. In some embodiments, the VL is selected from the group consisting of: SEQ ID NO: 1002, SEQ ID NO:984, and SEQ ID Nos:630-735 or a variant thereof.

In certain embodiments, the CD28 ABD is capable of binding to the CD28, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1) at detectable limits of the assay.

In another exemplary embodiment, the CD28 ABD include the variable heavy (VH) domain and/or variable light (VL) domain of any one of the CD28 ABDs described herein, including the figures and sequence listing. In exemplary embodiments, the CD28 ABD is one of the following CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing). Any of the CD28 ABD variable heavy domains or variants thereof disclosed herein can be combined with any of the variable light domains or variants thereof described herein to form CD28 ABDs for use in any of the antibody formats provided herein.

In addition to the parental CD28 variable heavy and variable light domains disclosed herein, provided herein are CD28 ABDs that include a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing). In certain embodiments, the CD28 ABD is capable of binding to CD28, as measured at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the variant VH and/or VL domain is at least 90, 95, 97, 98 or 99% identical to the VH and/or VL of a CD28 ABD as described herein, including the figures and sequence listing. In exemplary embodiments, the variant VH and/or VL domain is at least 90, 95, 97, 98 or 99% identical to the VH and/or VL of one of the following CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing). In certain embodiments, the CD28 ABD is capable of binding to CD28, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the CD28 antigen binding domain includes a variable heavy domain (VH) having the vhCDR1-3 (i.e., vhCDR1-3) of 1A7_H1.14 (FIG. 19). In some embodiments, the CD28 antigen binding domain further includes any of the CD28 binding domain variable light domains provided herein. In exemplary embodiments, the variable light domain is 1A7_L1.71 (FIG. 20) or a variant thereof. In certain embodiments, the CD28 ABD is capable of binding CD28 antigen, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1). Such CD28 binding domains can be included in any of the antibodies provided herein including, for example, “1+1 Fab-scFv-Fc,” “2+1 Fab₂-scFv-Fc,” “2+1 mAb-scFv,” “1+1 common light chain,” and “2+1 common light chain” antibodies.

In one embodiment, the CD28 ABD includes a variable heavy domain (VH) having vhCDR1-3s with 1, 2, 3, 4, 5, 6, 7, 8, 9, 10 amino acid modifications as compared to the vhCDR1-3 of 1A7_H1.14 (FIG. 19). In some embodiments, the CD28 antigen binding domain further includes any of the CD28 binding domain variable light domains provided herein. In exemplary embodiments, the variable light domain is 1A7_L1.71 (FIG. 20) or a variant thereof. In certain embodiments, the CD28 ABD is capable of binding CD28 antigen, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1). Such CD28 binding domains can be included in any of the antibodies provided herein including, for example, “1+1 Fab-scFv-Fc,” “2+1 Fab₂-scFv-Fc,” “2+1 mAb-scFv,” “1+1 common light chain,” and “2+1 common light chain” antibodies.

In some embodiments, the CD28 ABD includes a variable heavy domain (VH) having vhCDR1-3s that are at least 90, 95, 97, 98 or 99% identical to the 6 vhCDR1-3 of 1A7_H1.14 (FIG. 19). In some embodiments, the CD28 antigen binding domain further includes any of the CD28 binding domain variable light domains provided herein. In exemplary embodiments, the variable light domain is 1A7_L1.71 (FIG. 20) or a variant thereof. In certain embodiments, the CD28 ABD is capable of binding to the CD28, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1). Such CD28 binding domains can be included in any of the antibodies provided herein including, for example, “1+1 Fab-scFv-Fc,” “2+1 Fab₂-scFv-Fc,” “2+1 mAb-scFv,” “1+1 common light chain,” and “2+1 common light chain” antibodies.

In another exemplary embodiment, the CD28 ABD include the variable heavy (VH) domain 1A7_H1.14 (FIG. 19). In some embodiments, the CD28 antigen binding domain further includes any of the CD28 binding domain variable light domains provided herein. In exemplary embodiments, the variable light domain is 1A7_L1.14 (FIG. 20) or a variant thereof. In certain embodiments, the CD28 ABD is capable of binding to the CD28, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1). Such CD28 binding domains can be included in any of the antibodies provided herein including, for example, “1+1 Fab-scFv-Fc,” “2+1 Fab₂-scFv-Fc,” “2+1 mAb-scFv,” “1+1 common light chain,” and “2+1 common light chain” antibodies.

In addition to the parental CD28 variable heavy domains disclosed herein, provided herein are CD28 ABDs that include a variable heavy domain that is a variant of 1A7_H1.14 (FIG. 19). In one embodiment, the variant VH domain has from 1, 2, 3, 4, 5, 6, 7, 8, 9 or 10 amino acid changes from 1A7_H1.14 (FIG. 19). In some embodiments, the CD28 antigen binding domain further includes any of the CD28 binding domain variable light domains provided herein. In exemplary embodiments, the variable light domain is 1A7_L1.71 (FIG. 20) or a variant thereof. In certain embodiments, the CD28 ABD is capable of binding to CD28, as measured at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1). Such CD28 binding domains can be included in any of the antibodies provided herein including, for example, “1+1 Fab-scFv-Fc,” “2+1 Fab₂-scFv-Fc,” “2+1 mAb-scFv,” “1+1 common light chain,” and “2+1 common light chain” antibodies.

In one embodiment, the variant VH domain is at least 90, 95, 97, 98 or 99% identical to 1A7_H1.14 (FIG. 19). In some embodiments, the CD28 antigen binding domain further includes any of the CD28 binding domain variable light domains provided herein. In exemplary embodiments, the variable light domain is 1A7_L1.71 (FIG. 20) or a variant thereof. In certain embodiments, the CD28 ABD is capable of binding to CD28, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 antigen (see FIG. 1). Such CD28 binding domains can be included in any of the antibodies provided herein including, for example, “1+1 Fab-scFv-Fc,” “2+1 Fab₂-scFv-Fc,” “2+1 mAb-scFv,” “1+1 common light chain,” and “2+1 common light chain” antibodies.

In some embodiments, the CD28 antigen binding domain provided herein includes one or more of the sequences depicted in FIG. 70. In some embodiments, the CD28 antigen binding domain includes at least one of the HCDR1-3 and/or LCDR1-3 sequences of FIG. 70. In some embodiments, the CD28 antigen binding domain includes at least one of the HFR1-4 and/or LFR1-4 sequences of FIG. 70.

Specific anti-CD28 ABDs of interest include a VH domain with an amino acid sequence selected from the group consisting of SEQ ID NO:998, SEQ ID NO:984, SEQ ID NO:994 and SEQ ID NOs:561-629 or a variant thereof, paired with a VL domain of SEQ ID NO:1002, SEQ ID NO:984, and SEQ ID Nos:630-735 or a variant thereof.

In some cases, the VH and VL of the CD28 binding domain is selected from the following VH and VLs, respectively: SEQ ID NO:1030 and SEQ ID NO:1034; SEQ ID NO:1006 and SEQ ID NO:1010; SEQ ID NO:1014 and SEQ ID NO:1018; and SEQ ID NO:1022 and SEQ ID NO:1026

In other cases, the VH and VL of the CD28 binding domain is selected from the following VH and VLs, respectively: SEQ ID NOs:1 and 5, SEQ ID NOs:9 and 13, SEQ ID NOs:17 and 21, SEQ ID NOs:25 and 29, SEQ ID NOs:33 and 37, SEQ ID NOs:41 and 46, SEQ ID NOs:49 and 53, SEQ ID NOs:57 and 61, SEQ ID NOs:65 and 69, SEQ ID NOs:73 and 77, and SEQ ID NOs:81 and 85. or variants thereof.

### V. Antibodies

In one aspect provided herein are anti-CD28 antibodies and anti-PD-L1 antibodies. Antibodies provided herein can include any of the PD-L1 and/or CD28 binding domains provided herein (e.g., “1+1 Fab-scFv-Fc,” “2+1 Fab₂-scFv-Fc,” “2+1 mAb-scFv,” “1+1 common light chain,” and “2+1 common light chain” antibodies).

The antibodies provided herein include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

As shown herein, there are a number of suitable linkers (for use as either domain linkers or scFv linkers) that can be used to covalently attach the recited domains (e.g., scFvs, Fabs, Fc domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KTR), hinge region-derived sequences, and other natural sequences from other proteins.

In some embodiments, the linker is a “domain linker”, used to link any two domains as outlined herein together. For example, in the 2+1 Fab₂-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 7.

In some embodiments, the linker is a scFv linker that is used to covalently attach the VH and VL domains as discussed herein. In many cases, the scFv linker is a charged scFv linker, a number of which are shown in FIG. 6. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the invention as well, and thus those included in FIG. 6 can be used in any embodiment herein where a linker is utilized.

The PD-L1 binding domains and CD28 binding domains provided can be included in any useful antibody format including, for example, canonical immunoglobulin, as well as the “1+1 Fab-scFv-Fc,” “2+1 Fab₂-scFv-Fc,” “2+1 mAb-scFv,” “1+1 common light chain,” and “2+1 common light chain” formats provided herein (see, e.g., FIGS. 26 and 27). Other useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in US20180127501A1, which is incorporated by reference herein, particularly in pertinent part relating to antibody formats (see, e.g., FIG. 2).

In some embodiments, the subject antibody includes one or more of the PD-L1 ABDs provided herein. In some embodiments, the antibody includes one PD-L1 ABD. In other embodiments, the antibody includes two PD-L1 ABDs.

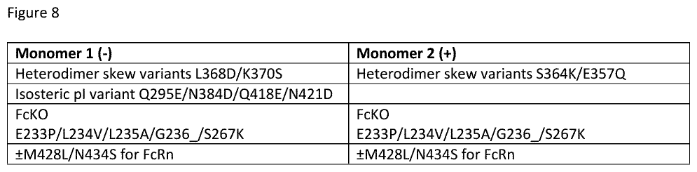

In an exemplary embodiment, the antibody is a bispecific antibody that includes one or two PD-L1 ABDs, including any of the PD-L1 ABDs provided herein. Bispecific antibody that include such PD-L1 ABDs include, for example, “1+1 Fab-scFv-Fc,” “2+1 Fab₂-scFv-Fc,” “2+1 mAb-scFv,” “1+1 common light chain,” and “2+1 common light chain” bispecifics format antibodies (FIGS. 26 and 27). In exemplary embodiments, the PD-L1 ABD is selected from those in FIGS. 28-31, as well as FIGS. 15, 73 and 78 of U.S. Pat. No. 10,793,632, as well as SEQ ID NOs:3961 to 4432 of that application. In exemplary embodiments, the PD-L1 ABD is one of the following 2G4 ABDs: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31). In exemplary embodiments the PD-L1 binding domains is a Fab. In some embodiments, such bispecific antibodies are heterodimeric bispecific antibodies that include any of the heterodimerization skew variants, pI variants and/or ablation variants described herein. See FIG. 8.

In some embodiments, the subject antibody includes one or more of the CD28 ABDs provided herein. In some embodiments, the antibody includes one CD28 ABD. In other embodiments, the antibody includes two CD28 ABDs. In exemplary embodiments, the antibody includes the variable heavy domain and variable light domain of one of the CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing).

In an exemplary embodiment, the antibody is a bispecific antibody that includes one or two CD28 ABDs, including any of the CD28 ABDs provided herein. Bispecific antibody that include such CD28 ABDs include, for example, “1+1 Fab-scFv-Fc,” “2+1 Fab₂-scFv-Fc,” “2+1 mAb-scFv,” “1+1 common light chain,” and “2+1 common light chain” bispecifics format antibodies (FIG. 21). In exemplary embodiments, the CD28 ABD is one of the following CD28 ABDs: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing). In exemplary embodiments, the CD28 ABD is an anti-CD28 scFv included in an “1+1 Fab-scFv-Fc,” or “2+1 Fab₂-scFv-Fc bispecifics format antibodies (FIG. 21). In some embodiments, such bispecific antibodies are heterodimeric bispecific antibodies that include any of the heterodimerization skew variants, pI variants and/or ablation variants described herein. See FIG. 8.

### A. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### B. Anti-CD28×Anti-PD-L1 Antibodies

In another aspect, provided herein are anti-CD28×anti-PD-L1 antibodies. In some embodiments, the anti-CD28×anti-PD-L1 antibody includes an agonistic CD28 binding domain that provides co-stimulatory function by binding to CD28 on T cells. As such, the anti-CD28×anti-PD-L1 antibody provided herein enhance immune responses selectively at tumor sites that express PD-L1. In some embodiments, the anti-CD28×anti-PD-L1 antibody is a bispecific antibody. In some embodiments, the anti-CD28×anti-PD-L1 antibody is a trispecific antibody. In some embodiments, the anti-CD28×anti-PD-L1 antibody is a bivalent antibody. In some embodiments, the anti-CD28×anti-PD-L1 antibody is a trivalent antibody. In some embodiments, the anti-CD28×anti-PD-L1 antibody is a bispecific, bivalent antibody. In exemplary embodiments, the anti-CD28×anti-PD-L1 antibody is a bispecific, trivalent antibody.

As is more fully outlined herein, the anti-CD28×anti-PD-L1 antibody can be in a variety of formats, as outlined below. Exemplary formats include the “1+1 Fab-scFv-Fc,” “2+1 Fab₂-scFv-Fc,” “1+1 common light chain,” and “2+1 common light chain” formats provided herein (see, e.g., FIGS. 26 and 27). Other useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in US20180127501A1, which is incorporated by reference herein, particularly in pertinent part relating to antibody formats (see, e.g., FIG. 2).

The -anti-CD28×anti-PD-L1 antibody can include any suitable CD28 ABD, including those described herein. In some embodiments, the CD28 ABD is an agonistic ABD that provides co-stimulatory function upon binding to CD28. In some embodiments, the anti-CD28×anti-TAA antibody includes a CD28 binding domain that includes the variable heavy domain and variable light of one of the following CD28 binding domains or variant thereof: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing).

The anti-CD28×anti-PD-L1 antibody provided herein can include one or more PD-L1 binding domains. In some embodiments, the anti-CD28×anti-PD-L1 antibody includes one PD-L1 binding domain. In certain embodiments, the anti-CD28×anti-PD-L1 antibody includes two PD-L1 binding domains.

Note that unless specified herein, the order of the antigen list in the name does not confer structure; that is an anti-PD-L1×anti-CD28 1+1 Fab-scFv-Fc antibody can have the scFv bind to PD-L1 or CD28, although in some cases, the order specifies structure as indicated.

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain).

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain). The scFv sequences contained in the sequence listing utilize a particular charged linker, but as outlined herein, uncharged or other charged linkers can be used, including those depicted in FIG. 6.

In addition, as discussed above, the numbering used in the Sequence Listing for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85 or 90% identity to a human germline sequence selected from those listed in FIG. 1 of U.S. Pat. No. 7,657,380.

### C. Heterodimeric Antibodies

In exemplary embodiments, the αPD-L1 antibodies provided herein are heterodimeric bispecific antibodies (e.g., αPD-L1×αCD28 heterodimeric bispecific antibodies) that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

1. Skew Variants

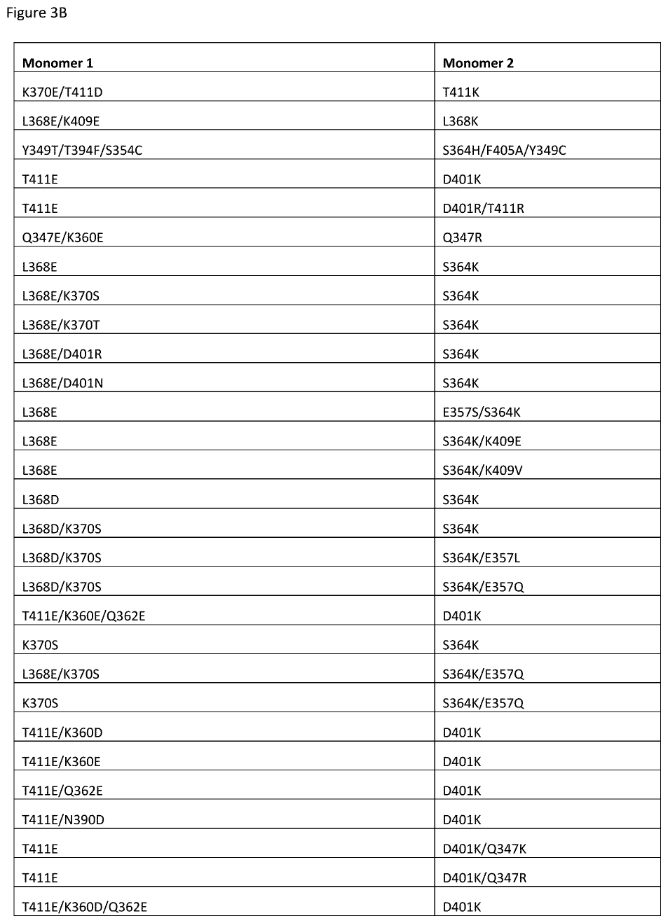

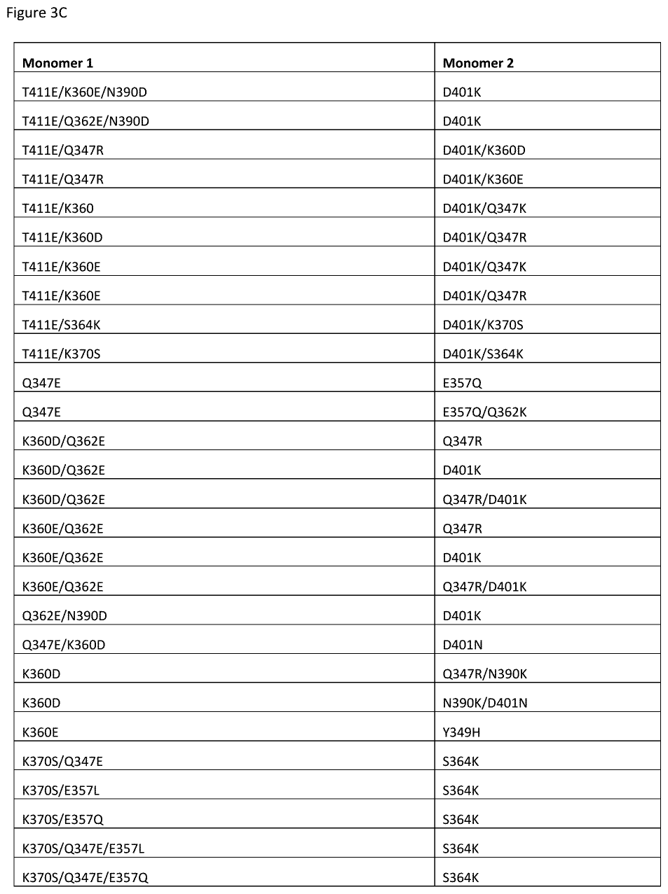

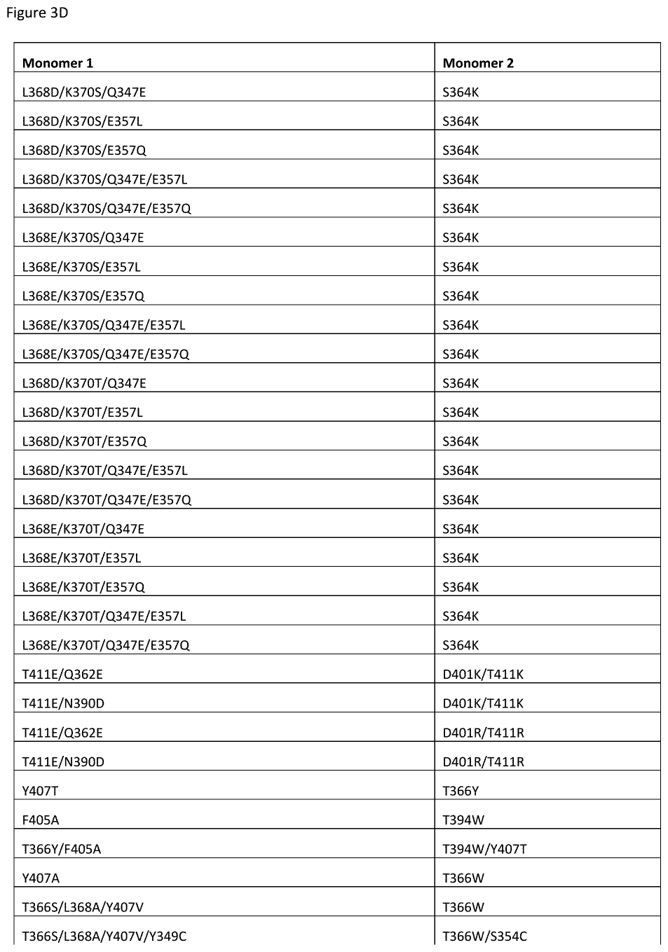

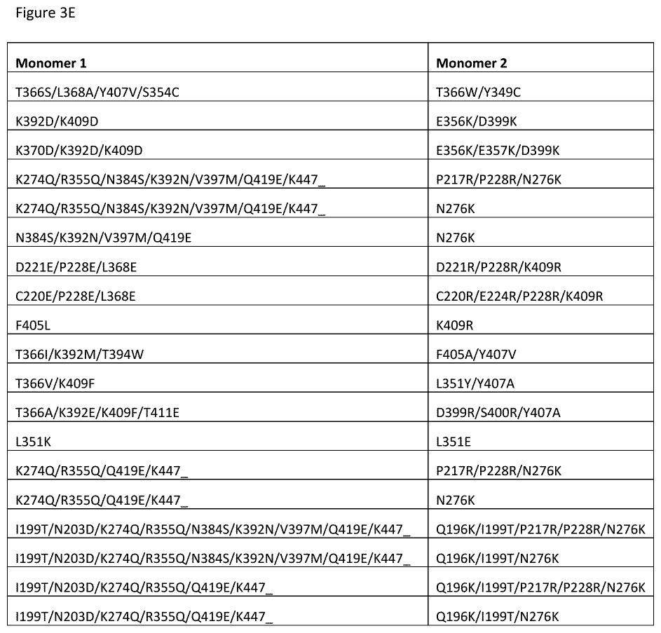

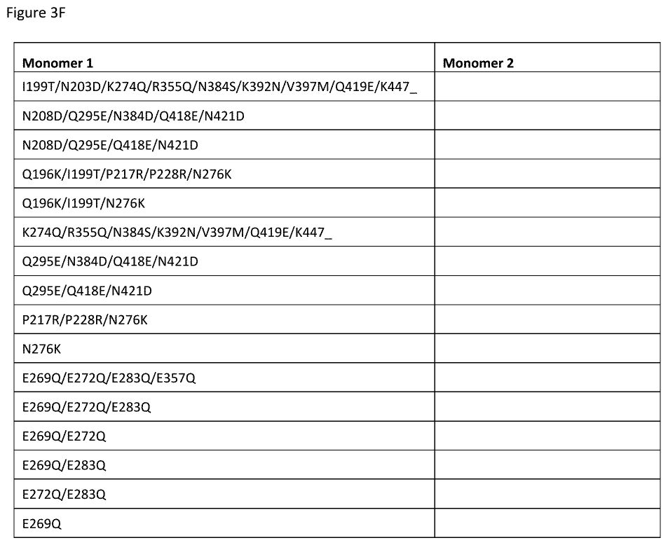

In some embodiments, the αPD-L1 heterodimeric antibody (e.g., αPD-L1×αCD28 heterodimeric antibody) includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 3, 8 and 9.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIGS. 3, 8, and 9. In exemplary embodiments, the heterodimeric antibody includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C) “skew” variant amino acid substitution set. In an exemplary embodiment, the heterodimeric antibody includes a “S364K/E357Q: L368D/K370S” amino acid substitution set. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

2. pI (Isoelectric Point) Variants for Heterodimers

In some embodiments, the αPD-L1 heterodimeric antibody (e.g., αPD-L1×αCD28 heterodimeric antibody) includes purification variants that advantageously allow for the separation of heterodimeric antibody from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, the “2+1 Fab₂-scFv-Fc” format, and the “2+1 CLC” format allows separation on the basis of size. As described above, it is also possible to “skew” the formation of heterodimers over homodimers using skew variants. Thus, a combination of heterodimerization skew variants and pI variants find particular use in the heterodimeric antibodies provided herein.

Additionally, as more fully outlined below, depending on the format of the heterodimeric antibody, pI variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab₂-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

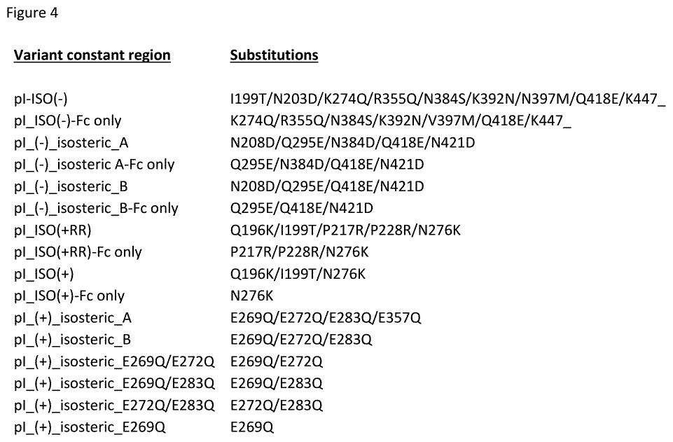

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine.). A number of these variants are shown in the FIGS. 3 and 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab₂-scFv-Fc, 1+1 CLC and 2+1 CLC formats, the starting pI of the scFv (1+1 Fab-scFv-Fc, 2+1 Fab₂-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. Preferred combinations of pI variants are shown in FIGS. 3 and 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO:892). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 4 and the other monomer has a charged linker (either in the form of a charged scFv linker because that monomer comprises an scFv or a charged domain linker, as the format dictates, which can be selected from those depicted in FIG. 6).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an anti-CD28 or anti-PD-L1 antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389, 392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is more further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### D. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

1. FcγR Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 436I or V/434S, 436V/428L and 259I/308F/428L. Such modification may be included in one or both Fc domains of the subject antibody.

2. Ablation Variants

In some embodiments, the heterodimeric antibody (e.g., anti-PD-L1×anti-CD28 bispecific antibody) includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 5, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### E. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants, skew and pI, are also independently and optionally combined with Fc ablation variants, Fc variants, FcRn variants, as generally outlined herein.

Exemplary combination of variants that are included in some embodiments of the heterodimeric 1+1 Fab-scFv-Fc, 2+1 Fab₂-scFv-Fc, 2+1 mAb-scFv, 1+1 CLC and 2+1 CLC format antibodies are included in FIG. 8. In some embodiments, the heterodimeric antibody includes a combination of variants as depicted in FIG. 8. In certain embodiments, the antibody is a heterodimeric 1+1 Fab-scFv-Fc, 2+1 Fab₂-scFv-Fc, +1 mAb-scFv, 1+1 CLC or 2+1 CLC format antibody.

### F. Useful Antibody Formats

As will be appreciated by those in the art and discussed more fully below, the heterodimeric bispecific antibodies provided herein can take on several different configurations as generally depicted in FIG. 21.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain.

In some embodiments, the present invention utilizes PD-L1 antigen binding domains. Any of the anti-PD-L1 antigen binding domains can be used, whether CDRs, variable light and variable heavy domains, Fabs and scFvs as depicted herein, including in any of the Figures (e.g., FIGS. 28-31) and the Sequence Listing, can be used, optionally and independently combined in any combination.

In some embodiments, the present invention utilizes CD28 antigen binding domains in combination with PD-L1 binding domains. As is outlined herein, when CD28 is one of the target antigens, it is preferable that the CD28 is bound only monovalently. As will be appreciated by those in the art, any collection of anti-CD28 CDRs, anti-CD28 variable light and variable heavy domains, Fabs and scFvs as depicted herein, including in any of the Figures (see particularly FIGS. 18-23) and the Sequence Listing, can be used.

1. 1+1 Fab-scFv-Fc format (“Bottle Opener”)

One heterodimeric antibody format that finds particular use in subject bispecific antibodies provided herein (e.g., anti-CD28×anti-PD-L1 antibody) is the “1+1 Fab-scFv-Fc” or “bottle opener” format as shown in FIG. 26A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv”, as defined below) and a second Fc domain. The scFv includes a second variable heavy domain (VH2) and a second variable light domain (VL2), wherein the VH2 is attached to the VL2 using an scFv linker that can be charged (see, e.g., FIG. 6). The scFv is attached to the heavy chain using a domain linker (see, e.g., FIG. 7). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a PD-L1 binding domain. In some embodiments, the first binding domain (i.e., the Fab) is a PD-L1 binding domain. In certain embodiments, the second binding domain (i.e., the scFv) is the PD-L1 binding domain. Any suitable PD-L1 binding domain can be included in the subject antibody including those provided herein (FIGS. 28-31 and the Sequence Listing).

In some embodiments, the PD-L1 binding domain is one of the following PD-L1 binding domains or a variant thereof: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31 and the Sequence Listing).

In some embodiments of the 1+1 Fab-scFv-Fc format, the PD-L1 binding domain has a variable heavy domain (VH) selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and a variable light domain (VL) selected from the group consisting of: SEQ ID NO: 1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOS:798-868 or a variant thereof.

In some embodiments, the VH and VL of the PD-L1 binding domain are selected from the group consisting of: a VH having an amino acid sequence of SEQ ID NO: 1183 and a VL having an amino acid sequence of SEQ ID NO:1187; a VH having an amino acid of SEQ ID NO:1042 and a VL having an amino acid sequence of SEQ ID NO:1047; a VH having an amino acid sequence of SEQ ID NO: 1159 and a VL having an amino acid sequence of SEQ ID NO: 1163; a VH having an amino acid sequence of SEQ ID NO: 1167 and a VL having an amino acid sequence of SEQ ID NO:1171; a VH having an amino acid sequence of SEQ ID NO: 1175 and a VL having an amino acid sequence of SEQ ID NO: 1179; a VH having an amino acid sequence of SEQ ID NO: 1191 and a VL having an amino acid sequence of SEQ ID NO: 1195; a VH having an amino acid sequence of SEQ ID NO:1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

In some embodiments, the PD-L1 binding domain is selected from one of the following PD-L1 binding domains: 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SH1D1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, PDL1.3.1 or a variant thereof.

In some embodiments, the VH and VL of the PD-L1 binding domain is selected from the following VH and VLs, respectively: SEQ ID NOs:89 and 93, SEQ ID NOs:97 and 101, SEQ ID NOs:105 and 109, SEQ ID NOs:113 and 117, SEQ ID NOs:121 and 125, SEQ ID NOs:129 and 133, SEQ ID NOs:137 and 141, SEQ ID NOs:145 and 149, SEQ ID NOs:153 and 157, SEQ ID NOs:161 and 165, SEQ ID NOs:169 and 173, SEQ ID NOs:177 and 181, SEQ ID NOs:185 and 189, SEQ ID NOs:193 and 197, SEQ ID NOs:201 and 205, SEQ ID NOs:209 and 213, SEQ ID NOs:217 and 221, SEQ ID NOs:225 and 229, SEQ ID NOs:233 and 237, SEQ ID NOs:241 and 245, SEQ ID NOs:249 and 253, SEQ ID NOs:257 and 261, SEQ ID NOs:265 and 269, SEQ ID NOs:273 and 277, SEQ ID NOs:281 and 285, SEQ ID NOs:289 and 293, SEQ ID NOs:297 and 301, SEQ ID NOs:305 and 309, SEQ ID NOs:313 and 317, SEQ ID NOs:321 and 325, SEQ ID NOs:329 and 333, SEQ ID NOs:337 and 341, SEQ ID NOs:345 and 349, SEQ ID NOs:353 and 357, SEQ ID NOs:361 and 365, SEQ ID NOs:369 and 373, SEQ ID NOs:374 and 381, SEQ ID NOs:385 and 389, SEQ ID NOs:393 and 397, SEQ ID NOs:401 and 405, SEQ ID NOs:409 and 413, SEQ ID NOs:417 and 421, SEQ ID NOs:425 and 429, SEQ ID NOs:433 and 437, SEQ ID NOs:441 and 445, SEQ ID NOs:449 and 453, SEQ ID NOs:457 and 461, SEQ ID NOs:465 and 469, SEQ ID NOs:473 and 477, SEQ ID NOs:481 and 485, SEQ ID NOs:489 and 493, SEQ ID NOs:497 and 501, SEQ ID NOs:504 and 508, SEQ ID NOs:512 and 516, SEQ ID NOs:520 and 524, SEQ ID NOs:528 and 534, SEQ ID NOs:537 and 541, SEQ ID NOs:545 and 549, SEQ ID NOs:553 and 557 or variants thereof.

In some embodiments of the 1+1 Fab-scFv-Fc format antibody, one of the first or second antigen binding domain is a CD28 binding domain and the other binding domain is a PD-L1 binding domain. In some embodiments where the 1+1 Fab-scFv-Fc includes a CD28 binding domain and a PD-L1 binding domain, it is the scFv that binds to the CD28, and the Fab that binds PD-L1. Exemplary anti-PD-L1×anti-CD28 bispecific antibody in the 1+1 Fab-scFv-Fc format is depicted in FIG. 35. In some embodiments, the anti-PD-L1×anti-CD28 bispecific antibody is an antibody depicted in FIG. 35.

In some embodiments, the first and second Fc domains of the 1+1 Fab-scFv-Fc format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the CH1-hinge-CH2-CH3 of the first monomer comprises amino acid variants L368D/K370S/N208D/Q295E/N384D/Q418E/N421D/E233P/L234V/L235A/G236del/S267K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 6). In some embodiments, the 1+1 Fab-scFv-Fc format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker. In some embodiments, the 1+1 Fab-scFv-Fc format antibody provided herein includes FcRn variants M428L/N434S, wherein numbering is according to EU numbering.

In some embodiments, one of the first binding domain or the second binding domain binds CD28 and the other binding domain binds PD-L1. In some embodiments of the 1+1 Fab-scFv-Fc format antibody, the first antigen binding domain (i.e., the Fab binding domain) binds PD-L1 and the second antigen binding domain (i.e., the scFv) binds CD28. In some embodiments of the 1+1 Fab-scFv-Fc format antibody, the first antigen binding domain binds CD28 (i.e., the Fab binding domain) and the second antigen binding domain (i.e., the scFv) binds PD-L1.

Any suitable CD28 binding domain can be included in subject 1+1 Fab-scFv-Fc format antibody, including any of the CD28 binding domains provided herein (see FIGS. 18-23 and the Sequence listing). In some embodiments, the CD28 binding domain is one of the following CD28 binding domains or a variant thereof: 1A7[CD28]_H1L1, 1A7[CD28]_H1.14L1, 1A7[CD28]_L1H1.14, 1A7[CD28]_H1.1L1, 1A7[CD28]_H1L1.71, IA7[CD28]_H1.14L1.71, 1A7[CD28]_L1.71H1.14, 1A7[CD28]_H1.1L1.71, HuTN228[CD28]_H1L1, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, PV1[CD28]_H0L0, m9.3[CD28]_H0L0, and hu9.3[CD28]_H1L1 or a variant thereof (FIGS. 18-23 and the Sequence Listing).

In some embodiments of the 1+1 Fab-scFv-Fc format, the CD28 binding domain has a variable heavy domain (VH) selected from the group consisting of: SEQ ID NO:998, SEQ ID NO:984, SEQ ID NO:994 and SEQ ID NOs:561-629 or a variant thereof, and a variable light domain (VL) selected from the group consisting of: SEQ ID NO:1002, SEQ ID NO:984, and SEQ ID NOs:630-735 or a variant thereof. In some embodiments, the VH and VL of the CD28 binding domain are selected from the group consisting of: a VH having an amino acid sequence of SEQ ID NO:1030 and a VL having an amino acid sequence of SEQ ID NO:1034; a VH having an amino acid sequence of SEQ ID NO:1006 and a VL having an amino acid sequence of SEQ ID NO:1010; a VH having an amino acid sequence of SEQ ID NO:1014 and a VL having an amino acid sequence of SEQ ID NO:1018; and a VH having an amino acid sequence of SEQ ID NO:1022 and a VL having an amino acid sequence of SEQ ID NO:1026.

In exemplary embodiments, the CD28 binding domain includes a 1A7[CD28]_H1.14 variable heavy domain. In some embodiments, the CD28 binding domain includes a 1A7[CD28]_H1.14 variable heavy domain or variant thereof and a light variable domain of any of the CD28 binding domains provided herein. In exemplary embodiments, the CD28 binding domain is 1A7[CD28]_H1.14L1.71 or a variant thereof.

In some embodiments, the CD28 binding domain is selected from one of the following CD28 binding domains: CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1 or a variant thereof.

In some embodiments, the VH and VL of the CD28 binding domain is selected from the following VH and VLs, respectively: SEQ ID NOs:1 and 5, SEQ ID NOs:9 and 13, SEQ ID NOs:17 and 21, SEQ ID NOs:25 and 29, SEQ ID NOs:33 and 37, SEQ ID NOs:41 and 46, SEQ ID NOs:49 and 53, SEQ ID NOs:57 and 61, SEQ ID NOs:65 and 69, SEQ ID NOs:73 and 77, and SEQ ID NOs:81 and 85. or variants thereof.

Any suitable PD-L1 binding domain can be included in subject anti-CD28×anti-PD-L11+1 Fab-scFv-Fc format antibody, including any of the PD-L1 binding domains provided herein (see, e.g., FIGS. 28-31 and the Sequence Listing). In exemplary embodiments, the PD-L1 binding domain has a VH with an amino acid sequence of SEQ ID NO:1183 and a VL with an amino acid sequence of SEQ ID NO:1187. In exemplary embodiments, the PD-L1 binding domain has a VH with an amino acid sequence of SEQ ID NO:1042 and a VL with an amino acid sequence of SEQ ID NO:1046. In exemplary embodiments, the PD-L1 binding domain has a VH with an amino acid sequence of SEQ ID NO:1159 and a VL with an amino acid sequence of SEQ ID NO:1163.

FIG. 10 shows some exemplary Fc domain sequences that are useful in the 1+1 Fab-scFv-Fc format antibodies. The “monomer 1” sequences depicted in FIG. 10 typically refer to the Fc domain of the “Fab-Fc heavy chain” and the “monomer 2” sequences refer to the Fc domain of the “scFv-Fc heavy chain.” In addition, FIGS. 12-15 provides exemplary CH1-hinge domains, CH1 domains, and hinge domains that can be included in the first or second monomer of the 1+1 Fab-scFv-Fc format. Further, FIG. 16 provides useful CL sequences that can be used with this format. 2. 2+1 Fab₂-scFv-Fc format (Central-scFv format)

One heterodimeric antibody format that finds particular use in the subject bispecific antibodies provided herein (e.g., anti-PD-L1×anti-CD28×antibody) is the 2+1 Fab2-scFv-Fc format (also referred to as “central-scFv format”) shown in FIG. 26B. This antibody format includes three antigen binding domains: two Fab portions and an scFv that is inserted between the VH-CH1 and CH2-CH3 regions of one of the monomers. In some embodiments of this format, the two Fab portions each bind PD-L1. In some embodiments, the “extra” scFv domain binds CD28. In some embodiments, the 2+1 Fab₂-scFv-Fc format antibody is a trivalent antibody.

In some embodiments of the 2+1 Fab₂-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 7. This embodiment further utilizes a common light chain that includes a variable light domain (VL1) and a constant light domain (CL). The common light chain associates with the VH1-CH1 of the first and second monomers to form two identical Fabs. In some embodiments, the identical Fabs each bind PD-L1. As for many of the embodiments herein, these constructs can include skew variants, pI variants, ablation variants, additional Fc variants, etc. as desired and described herein.

In some embodiments, the first and second Fc domains of the 2+1 Fab₂-scFv-Fc format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab₂-scFv-Fc format antibody provided herein includes a charged scFv linker (including those shown in FIG. 6). In some embodiments, the 2+1 Fab₂-scFv-Fc format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab₂-scFv-Fc format antibody provided herein includes a (GKPGS)₄ charged scFv linker. In some embodiments, the 2+1 Fab₂-scFv-Fc format antibody provided herein includes FcRn variants M428L/N434S, wherein numbering is according to EU numbering.

In some embodiments of the 2+1 Fab₂-scFv-Fc format antibody, the two Fab domains are PD-L1 binding domains. In some embodiments, the scFv is the PD-L1 binding domain. Any suitable PD-L1 binding domain can be included in the subject 2+1 Fab₂-scFv-Fc format antibody including those provided herein (FIGS. 28-31 and the Sequence Listing).

In some embodiments, the PD-L1 binding domain is one of the following PD-L1 binding domains or a variant thereof: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31 and the Sequence Listing).

In some embodiments of the 2+1 Fab₂-scFv-Fc format antibody, the PD-L1 binding domain has a variable heavy domain (VH) selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and a variable light domain (VL) selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOS:798-868 or a variant thereof.

In some embodiments, the VH and VL of the PD-L1 binding domain are selected from the group consisting of: a VH having an amino acid sequence of SEQ ID NO: 1183 and a VL having an amino acid sequence of SEQ ID NO:1187; a VH having an amino acid of SEQ ID NO:1042 and a VL having an amino acid sequence of SEQ ID NO:1047; a VH having an amino acid sequence of SEQ ID NO:1159 and a VL having an amino acid sequence of SEQ ID NO:1163; a VH having an amino acid sequence of SEQ ID NO:1167 and a VL having an amino acid sequence of SEQ ID NO:1171; a VH having an amino acid sequence of SEQ ID NO:1175 and a VL having an amino acid sequence of SEQ ID NO:1179; a VH having an amino acid sequence of SEQ ID NO:1191 and a VL having an amino acid sequence of SEQ ID NO:1195; a VH having an amino acid sequence of SEQ ID NO:1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

In some embodiments, the PD-L1 binding domain is selected from one of the following PD-L1 binding domains: 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SH1D1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, PDL1.3.1 or a variant thereof.

In some embodiments, the VH and VL of the PD-L1 binding domain is selected from the following VH and VLs, respectively: SEQ ID NOs:89 and 93, SEQ ID NOs:97 and 101, SEQ ID NOs:105 and 109, SEQ ID NOs:113 and 117, SEQ ID NOs:121 and 125, SEQ ID NOs:129 and 133, SEQ ID NOs:137 and 141, SEQ ID NOs:145 and 149, SEQ ID NOs:153 and 157, SEQ ID NOs:161 and 165, SEQ ID NOs:169 and 173, SEQ ID NOs:177 and 181, SEQ ID NOs:185 and 189, SEQ ID NOs:193 and 197, SEQ ID NOs:201 and 205, SEQ ID NOs:209 and 213, SEQ ID NOs:217 and 221, SEQ ID NOs:225 and 229, SEQ ID NOs:233 and 237, SEQ ID NOs:241 and 245, SEQ ID NOs:249 and 253, SEQ ID NOs:257 and 261, SEQ ID NOs:265 and 269, SEQ ID NOs:273 and 277, SEQ ID NOs:281 and 285, SEQ ID NOs:289 and 293, SEQ ID NOs:297 and 301, SEQ ID NOs:305 and 309, SEQ ID NOs:313 and 317, SEQ ID NOs:321 and 325, SEQ ID NOs:329 and 333, SEQ ID NOs:337 and 341, SEQ ID NOs:345 and 349, SEQ ID NOs:353 and 357, SEQ ID NOs:361 and 365, SEQ ID NOs:369 and 373, SEQ ID NOs:374 and 381, SEQ ID NOs:385 and 389, SEQ ID NOs:393 and 397, SEQ ID NOs:401 and 405, SEQ ID NOs:409 and 413, SEQ ID NOs:417 and 421, SEQ ID NOs:425 and 429, SEQ ID NOs:433 and 437, SEQ ID NOs:441 and 445, SEQ ID NOs:449 and 453, SEQ ID NOs:457 and 461, SEQ ID NOs:465 and 469, SEQ ID NOs:473 and 477, SEQ ID NOs:481 and 485, SEQ ID NOs:489 and 493, SEQ ID NOs:497 and 501, SEQ ID NOs:504 and 508, SEQ ID NOs:512 and 516, SEQ ID NOs:520 and 524, SEQ ID NOs:528 and 534, SEQ ID NOs:537 and 541, SEQ ID NOs:545 and 549, SEQ ID NOs:553 and 557 or variants thereof.

In some embodiments, the 2+1 Fab₂-scFv-Fc format antibody binds PD-L1 and CD28. In some embodiments, the scFv of the second monomer of the 2+1 Fab₂-scFv-Fc format antibody is a CD28 binding domain and the VH1 of the first and second monomer and the VL1 of the common light chain each form PD-L1 binding domains. In some embodiments, the scFv is a PD-L1 binding domain and the VH1 of the first and second monomer and the VL1 of the common light chain each form CD28 binding domains.

Any suitable CD28 binding domain can be included in subject 2+1 Fab₂-scFv-Fc format antibody, including any of the CD28 binding domains provided herein (see, e.g., FIGS. 18-23 and the Sequence Listing). In some embodiments, the CD28 binding domain is one of the following CD28 binding domains or a variant thereof: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1 (FIGS. 18-23 and the Sequence Listing).

In some embodiments of the 2+1 Fab₂-scFv-Fc format antibody, the CD28 binding domain has a variable heavy domain (VH) selected from the group consisting of: SEQ ID NO:998, SEQ ID NO:984, SEQ ID NO:994 and SEQ ID NOs:561-629 or a variant thereof, and a variable light domain (VL) selected from the group consisting of: SEQ ID NO:1002, SEQ ID NO:984, and SEQ ID NOs:630-735 or a variant thereof. In some embodiments, the VH and VL of the CD28 binding domain are selected from the group consisting of: a VH having an amino acid sequence of SEQ ID NO:1030 and a VL having an amino acid sequence of SEQ ID NO:1034; a VH having an amino acid sequence of SEQ ID NO:1006 and a VL having an amino acid sequence of SEQ ID NO:1010; a VH having an amino acid sequence of SEQ ID NO:1014 and a VL having an amino acid sequence of SEQ ID NO:1018; and a VH having an amino acid sequence of SEQ ID NO:1022 and a VL having an amino acid sequence of SEQ ID NO:1026.

In exemplary embodiments, the CD28 binding domain includes a 1A7[CD28]_H1.14 variable heavy domain. In some embodiments, the CD28 binding domain includes a 1A7[CD28]_H1.14 variable heavy domain or variant thereof and a light variable domain of any of the CD28 binding domains provided herein. In exemplary embodiments, the CD28 binding domain is 1A7[CD28]_H1.14L1.71 or a variant thereof.

In some embodiments, the CD28 binding domain is selected from one of the following CD28 binding domains CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1 or a variant thereof.

In some embodiments, the VH and VL of the CD28 binding domain is selected from the following VH and VLs, respectively: SEQ ID NOs:1 and 5, SEQ ID NOs:9 and 13, SEQ ID NOs:17 and 21, SEQ ID NOs:25 and 29, SEQ ID NOs:33 and 37, SEQ ID NOs:41 and 46, SEQ ID NOs:49 and 53, SEQ ID NOs:57 and 61, SEQ ID NOs:65 and 69, SEQ ID NOs:73 and 77, and SEQ ID NOs:81 and 85. or variants thereof.

In some embodiments of the anti-PD-L1×anti-CD28 2+1 Fab₂-scFv-Fc format antibody, the VH1 of the first and second monomer and the VL1 of the common light chain of the 2+1 Fab₂-scFv-Fc format antibody each form a binding domain that binds PD-L1. Any suitable PD-L1 binding domain(s) can be included in the subject 2+1 Fab₂-scFv-Fc format antibody, including any of the PD-L1 antigen binding domains provided herein (see, e.g., FIGS. 28-31 and the Sequence Listing). In exemplary embodiments, the PD-L1 binding domain has a VH with an amino acid sequence of SEQ ID NO:1183 and a VL with an amino acid sequence of SEQ ID NO:1187. In exemplary embodiments, the PD-L1 binding domain has a VH with an amino acid sequence of SEQ ID NO:1042 and a VL with an amino acid sequence of SEQ ID NO:1046. In exemplary embodiments, the PD-L1 binding domain has a VH with an amino acid sequence of SEQ ID NO: 1159 and a VL with an amino acid sequence of SEQ ID NO: 1163.

FIG. 11 shows some exemplary Fc domain sequences that are useful with the 2+1 Fab2-scFv-Fc format. The “monomer 1” sequences depicted in FIG. 11 typically refer to the Fc domain of the “Fab-Fc heavy chain” and the “monomer 2” sequences refer to the Fc domain of the “Fab-scFv-Fc heavy chain.” In addition, FIGS. 12-15 provides exemplary CH1-hinge domains, CH1 domains, and hinge domains that can be included in the first or second monomer of the 2+1 Fab2-scFv-Fc format. Further, FIG. 16 provides useful CL sequences that can be used with this format.

Exemplary anti-PD-L1×anti-CD28×antibodies in the 2+1 Fab₂-scFv-Fc format are depicted in FIGS. 36 and 41. In some embodiments, the anti-PD-L1×anti-CD28×antibody is an antibody depicted in FIG. 36 or 41.

3. 1+1 CLC Format

One heterodimeric antibody format that finds particular use in subject bispecific antibodies provided herein (e.g., anti-PD-L1×anti-CD28 antibody) is the “1+1 Common Light Chain” or “1+1 CLC” format, which is depicted in FIG. 26C. The 1+1 CLC format antibody includes a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In such embodiments, the VL pairs with the VH1 to form a first binding domain with a first antigen binding specificity; and the VL pairs with the VH2 to form a second binding domain with a second antigen binding specificity. In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the 1+1 CLC format antibody provided herein further includes FcRn variants M428L/N434S, wherein numbering is according to EU numbering.

In some embodiments of the 1+1 CLC format antibody, one of the first or second antigen binding domain is a PD-L1 binding domain. Any suitable PD-L1 binding domain can be included in the subject antibody including those provided herein (FIGS. 28-31 and the Sequence Listing).

In some embodiments, the PD-L1 binding domain is one of the following PD-L1 binding domains or a variant thereof: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31 and the Sequence Listing).

In some embodiments of the 1+1 CLC format antibody, the PD-L1 binding domain has a variable heavy domain (VH) selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, and SEQ ID NO:1099; and a variable light domain (VL) selected from the group consisting of: SEQ ID NO: 1111, SEQ ID NO:1046, SEQ ID NO: 1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868. In some embodiments, the VH and VL of the PD-L1 binding domain are selected from the group consisting of: a VH having an amino acid sequence of SEQ ID NO: 1183 and a VL having an amino acid sequence of SEQ ID NO: 1187; a VH having an amino acid of SEQ ID NO:1042 and a VL having an amino acid sequence of SEQ ID NO:1047; a VH having an amino acid sequence of SEQ ID NO:1159 and a VL having an amino acid sequence of SEQ ID NO:1163; a VH having an amino acid sequence of SEQ ID NO:1167 and a VL having an amino acid sequence of SEQ ID NO:1171; a VH having an amino acid sequence of SEQ ID NO:1175 and a VL having an amino acid sequence of SEQ ID NO:1179; a VH having an amino acid sequence of SEQ ID NO:1191 and a VL having an amino acid sequence of SEQ ID NO:1195; a VH having an amino acid sequence of SEQ ID NO:1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

In some embodiments, the PD-L1 binding domain is selected from one of the following PD-L1 binding domains: 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SH1D1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, PDL1.3.1 or a variant thereof.

In some embodiments, the VH and VL of the PD-L1 binding domain is selected from the following VH and VLs, respectively: SEQ ID NOs:89 and 93, SEQ ID NOs:97 and 101, SEQ ID NOs:105 and 109, SEQ ID NOs:113 and 117, SEQ ID NOs:121 and 125, SEQ ID NOs:129 and 133, SEQ ID NOs:137 and 141, SEQ ID NOs:145 and 149, SEQ ID NOs:153 and 157, SEQ ID NOs:161 and 165, SEQ ID NOs:169 and 173, SEQ ID NOs:177 and 181, SEQ ID NOs:185 and 189, SEQ ID NOs:193 and 197, SEQ ID NOs:201 and 205, SEQ ID NOs:209 and 213, SEQ ID NOs:217 and 221, SEQ ID NOs:225 and 229, SEQ ID NOs:233 and 237, SEQ ID NOs:241 and 245, SEQ ID NOs:249 and 253, SEQ ID NOs:257 and 261, SEQ ID NOs:265 and 269, SEQ ID NOs:273 and 277, SEQ ID NOs:281 and 285, SEQ ID NOs:289 and 293, SEQ ID NOs:297 and 301, SEQ ID NOs:305 and 309, SEQ ID NOs:313 and 317, SEQ ID NOs:321 and 325, SEQ ID NOs:329 and 333, SEQ ID NOs:337 and 341, SEQ ID NOs:345 and 349, SEQ ID NOs:353 and 357, SEQ ID NOs:361 and 365, SEQ ID NOs:369 and 373, SEQ ID NOs:374 and 381, SEQ ID NOs:385 and 389, SEQ ID NOs:393 and 397, SEQ ID NOs:401 and 405, SEQ ID NOs:409 and 413, SEQ ID NOs:417 and 421, SEQ ID NOs:425 and 429, SEQ ID NOs:433 and 437, SEQ ID NOs:441 and 445, SEQ ID NOs:449 and 453, SEQ ID NOs:457 and 461, SEQ ID NOs:465 and 469, SEQ ID NOs:473 and 477, SEQ ID NOs:481 and 485, SEQ ID NOs:489 and 493, SEQ ID NOs:497 and 501, SEQ ID NOs:504 and 508, SEQ ID NOs:512 and 516, SEQ ID NOs:520 and 524, SEQ ID NOs:528 and 534, SEQ ID NOs:537 and 541, SEQ ID NOs:545 and 549, SEQ ID NOs:553 and 557 or variants thereof.

In some embodiments, the 1+1 CLC format antibody is a bispecific antibody that binds PD-L1 and CD28. In some embodiments, one of the first binding domain or the second binding domain binds CD28 and the other binding domain binds PD-L1. Any suitable CD28 binding domain can be included in subject 1+1 CLC format antibody, including any of the CD28 binding domains provided herein. In some embodiments, the CD28 binding domain is one of the following CD28 binding domains or a variant thereof: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1 (FIGS. 18-23 and the Sequence Listing).

In some embodiments of the 1+1 CLC format antibody, the CD28 binding domain has a variable heavy domain (VH) selected from the group consisting of: SEQ ID NO:998, SEQ ID NO:984, SEQ ID NO:994 and SEQ ID NOs:561-629 or a variant thereof, and a variable light domain (VL) selected from the group consisting of: SEQ ID NO:1002, SEQ ID NO:984, and SEQ ID NOs:630-735 or a variant thereof. In some embodiments, the VH and VL of the CD28 binding domain are selected from the group consisting of: a VH having an amino acid sequence of SEQ ID NO:1030 and a VL having an amino acid sequence of SEQ ID NO:1034; a VH having an amino acid sequence of SEQ ID NO:1006 and a VL having an amino acid sequence of SEQ ID NO:1010; a VH having an amino acid sequence of SEQ ID NO:1014 and a VL having an amino acid sequence of SEQ ID NO:1018; and a VH having an amino acid sequence of SEQ ID NO:1022 and a VL having an amino acid sequence of SEQ ID NO:1026.

In exemplary embodiments, the CD28 binding domain includes a 1A7[CD28]_H1.14 variable heavy domain. In some embodiments, the CD28 binding domain includes a 1A7[CD28]_H1.14 variable heavy domain or variant thereof and a light variable domain of any of the CD28 binding domains provided herein. In exemplary embodiments, the CD28 binding domain is 1A7[CD28]_H1.14L1.71 or a variant thereof.

In some embodiments, the CD28 binding domain is selected from one of the following CD28 binding domains CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H11L1 or a variant thereof.

In some embodiments, the VH and VL of the CD28 binding domain is selected from the following VH and VLs, respectively: SEQ ID NOs:1 and 5, SEQ ID NOs:9 and 13, SEQ ID NOs:17 and 21, SEQ ID NOs:25 and 29, SEQ ID NOs:33 and 37, SEQ ID NOs:41 and 46, SEQ ID NOs:49 and 53, SEQ ID NOs:57 and 61, SEQ ID NOs:65 and 69, SEQ ID NOs:73 and 77, and SEQ ID NOs:81 and 85. or variants thereof.

In some embodiments, one of the first binding domain or the second binding domain of the 1+1 CLC format antibody binds PD-L1. Any suitable PD-L1 antigen binding domain can be included, including those described herein (see, e.g., FIGS. 28-31 and the Sequence Listing). In some embodiments, the 1+1 CLC format antibody includes a PD-L1 antigen binding domain that includes a variable heavy domain of one of the PD-L1 antigen binding domains provided herein (see, e.g., FIGS. 28-31 and the Sequence Listing). In exemplary embodiments, the PD-L1 binding domain has a VH with an amino acid sequence of SEQ ID NO:1183 and a VL with an amino acid sequence of SEQ ID NO:1187. In exemplary embodiments, the PD-L1 binding domain has a VH with an amino acid sequence of SEQ ID NO:1042 and a VL with an amino acid sequence of SEQ ID NO:1046. In exemplary embodiments, the PD-L1 binding domain has a VH with an amino acid sequence of SEQ ID NO:1159 and a VL with an amino acid sequence of SEQ ID NO:1163.

4. 2+1 CLC Format

Another heterodimeric antibody format that finds particular use in subject bispecific antibodies provided herein (e.g., anti-PD-L1×anti-CD28 antibody) is the “2+1 Common Light Chain” or “2+1 CLC” format, which is depicted in FIG. 26D. The 2+1 CLC format includes a first monomer that includes a VH1-CH1-linker-VH1-CH1-hinge-CH2-CH3, wherein the VH1s are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. The VL pairs with each of the VH1s of the first monomer to form two first binding domains, each with a first antigen binding specificity; and the VL pairs with the VH2 to form a second binding domain with a second antigen binding specificity. The linker of the first monomer can be any suitable linker, including any one of the domain linkers or combinations thereof described in FIG. 7. In some embodiments, the linker is EPKSCGKPGSGKPGS (SEQ ID NO:936). In some embodiments, the 2+1 CLC format antibody is a trivalent antibody.

In some embodiments, the first and second Fc domains of the 2+1 CLC format are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments of the 2+1 CLC format antibody, each of the two first binding domains is a PD-L1 binding domain. In some embodiments, the second binding domain is a PD-L1 binding domain. Any suitable PD-L1 binding domain can be included in the subject antibody including those provided herein (see, e.g., FIGS. 28-31 and the Sequence Listing).

In some embodiments, the PD-L1 binding domain is one of the following PD-L1 binding domains or a variant thereof: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31 and the Sequence Listing).

In some embodiments of the 2+1 CLC format antibody, the PD-L1 binding domain has a variable heavy domain (VH) selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and a variable light domain (VL) selected from the group consisting of: SEQ ID NO: 1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof. In some embodiments, the VH and VL of the PD-L1 binding domain are selected from the group consisting of: a VH having an amino acid sequence of SEQ ID NO: 1183 and a VL having an amino acid sequence of SEQ ID NO: 1187; a VH having an amino acid of SEQ ID NO:1042 and a VL having an amino acid sequence of SEQ ID NO:1047; a VH having an amino acid sequence of SEQ ID NO:1159 and a VL having an amino acid sequence of SEQ ID NO: 1163; a VH having an amino acid sequence of SEQ ID NO: 1167 and a VL having an amino acid sequence of SEQ ID NO:1171; a VH having an amino acid sequence of SEQ ID NO: 1175 and a VL having an amino acid sequence of SEQ ID NO: 1179; a VH having an amino acid sequence of SEQ ID NO: 1191 and a VL having an amino acid sequence of SEQ ID NO: 1195; a VH having an amino acid sequence of SEQ ID NO: 1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

In some embodiments, the PD-L1 binding domain is selected from one of the following PD-L1 binding domains: 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SH1D1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, PDL1.3.1 or a variant thereof.

In some embodiments, the VH and VL of the PD-L1 binding domain is selected from the following VH and VLs, respectively: SEQ ID NOs:89 and 93, SEQ ID NOs:97 and 101, SEQ ID NOs:105 and 109, SEQ ID NOs:113 and 117, SEQ ID NOs:121 and 125, SEQ ID NOs:129 and 133, SEQ ID NOs:137 and 141, SEQ ID NOs:145 and 149, SEQ ID NOs:153 and 157, SEQ ID NOs:161 and 165, SEQ ID NOs:169 and 173, SEQ ID NOs:177 and 181, SEQ ID NOs:185 and 189, SEQ ID NOs:193 and 197, SEQ ID NOs:201 and 205, SEQ ID NOs:209 and 213, SEQ ID NOs:217 and 221, SEQ ID NOs:225 and 229, SEQ ID NOs:233 and 237, SEQ ID NOs:241 and 245, SEQ ID NOs:249 and 253, SEQ ID NOs:257 and 261, SEQ ID NOs:265 and 269, SEQ ID NOs:273 and 277, SEQ ID NOs:281 and 285, SEQ ID NOs:289 and 293, SEQ ID NOs:297 and 301, SEQ ID NOs:305 and 309, SEQ ID NOs:313 and 317, SEQ ID NOs:321 and 325, SEQ ID NOs:329 and 333, SEQ ID NOs:337 and 341, SEQ ID NOs:345 and 349, SEQ ID NOs:353 and 357, SEQ ID NOs:361 and 365, SEQ ID NOs:369 and 373, SEQ ID NOs:374 and 381, SEQ ID NOs:385 and 389, SEQ ID NOs:393 and 397, SEQ ID NOs:401 and 405, SEQ ID NOs:409 and 413, SEQ ID NOs:417 and 421, SEQ ID NOs:425 and 429, SEQ ID NOs:433 and 437, SEQ ID NOs:441 and 445, SEQ ID NOs:449 and 453, SEQ ID NOs:457 and 461, SEQ ID NOs:465 and 469, SEQ ID NOs:473 and 477, SEQ ID NOs:481 and 485, SEQ ID NOs:489 and 493, SEQ ID NOs:497 and 501, SEQ ID NOs:504 and 508, SEQ ID NOs:512 and 516, SEQ ID NOs:520 and 524, SEQ ID NOs:528 and 534, SEQ ID NOs:537 and 541, SEQ ID NOs:545 and 549, SEQ ID NOs:553 and 557 or variants thereof.

In some embodiments, the 2+1 CLC format antibody is a bispecific antibody that binds PD-L1 and CD28. In some embodiments, each of the two first binding domains binds PD-L1 and the second binding domain binds CD28. In some embodiments, each of the two first binding domains binds CD28 and the second binding domain binds PD-L1. Any suitable CD28 binding domain can be included in the subject 2+1 CLC format antibody, including any of the CD28 binding domains provided herein. In some embodiments, the CD28 binding domain is one of the following CD28 binding domains or a variant thereof: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1 (see, e.g., FIGS. 18-23 and the Sequence Listing). In some embodiments of the 2+1 CLC format antibody, the CD28 binding domain has a variable heavy domain (VH) selected from the group consisting of: SEQ ID NO:998, SEQ ID NO:984, SEQ ID NO:994 and SEQ ID NOs:561-629 or a variant thereof, and a variable light domain (VL) selected from the group consisting of: SEQ ID NO:1002, SEQ ID NO:984, and SEQ ID NOs:630-735 or a variant thereof. In some embodiments, the VH and VL of the CD28 binding domain are selected from the group consisting of: a VH having an amino acid sequence of SEQ ID NO:1030 and a VL having an amino acid sequence of SEQ ID NO:1034; a VH having an amino acid sequence of SEQ ID NO:1006 and a VL having an amino acid sequence of SEQ ID NO:1010; a VH having an amino acid sequence of SEQ ID NO:1014 and a VL having an amino acid sequence of SEQ ID NO:1018; and a VH having an amino acid sequence of SEQ ID NO:1022 and a VL having an amino acid sequence of SEQ ID NO:1026.

In exemplary embodiments, the CD28 binding domain includes a 1A7[CD28]_H1.14 variable heavy domain. In some embodiments, the CD28 binding domain includes a 1A7[CD28]_H1.14 variable heavy domain or variant thereof and a light variable domain of any of the CD28 binding domains provided herein. In exemplary embodiments, the CD28 binding domain is 1A7[CD28]_H1.14L1.71 or a variant thereof.

In some embodiments, the CD28 binding domain is selected from one of the following CD28 binding domains CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1 or a variant thereof.

In some embodiments, the VH and VL of the CD28 binding domain is selected from the following VH and VLs, respectively: SEQ ID NOs:1 and 5, SEQ ID NOs:9 and 13, SEQ ID NOs:17 and 21, SEQ ID NOs:25 and 29, SEQ ID NOs:33 and 37, SEQ ID NOs:41 and 46, SEQ ID NOs:49 and 53, SEQ ID NOs:57 and 61, SEQ ID NOs:65 and 69, SEQ ID NOs:73 and 77, and SEQ ID NOs:81 and 85. or variants thereof.

Any suitable PD-L1 binding domain can be included in the subject anti-PD-L1×anti-CD28 2+1 CLC format antibody, including any of the PD-L1 antigen binding domains provided herein (see, e.g., FIGS. 28-31 and the Sequence Listing). In exemplary embodiments, the PD-L1 binding domain(s) has a VH with an amino acid sequence of SEQ ID NO:1183 and a VL with an amino acid sequence of SEQ ID NO: 1187. In exemplary embodiments, the PD-L1 binding domain has a VH with an amino acid sequence of SEQ ID NO:1042 and a VL with an amino acid sequence of SEQ ID NO:1046. In exemplary embodiments, the PD-L1 binding domain has a VH with an amino acid sequence of SEQ ID NO:1159 and a VL with an amino acid sequence of SEQ ID NO:1163.

FIG. 13 depicts sequences for “CH1+half hinge” domain linker that find use in embodiments of the 2+1 CLC format. In the 2+1 CLC format, the “CH1+half hinge” sequences find use linking the first variable heavy domain (VH) to the second V_{H} domain on the Fab-Fab-Fc side of the bispecific antibody.

5. 2+1 mAb-scFv Format

One heterodimeric antibody format that finds particular use in the subject bispecific antibodies provided herein (e.g., anti-PD-L1×anti-CD28 antibody) is the 2+1 mAb-scFv format shown in FIG. 26E. This antibody format includes three antigen binding domains: two Fab portions and an scFv that is attached to the C-terminal of one of the heavy chains. In some embodiments of this format, the Fab portions each bind PD-L1. In some embodiments, the “extra” scFv domain binds CD28. That is, this mAb-scFv format is a trivalent antibody.

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In these embodiments, the VH1-VL1 each form a first ABD and the VH2-VL2 form a second ABD. In some embodiments, the first ABD binds to a tumor target antigen, including human B7H3, and the second ABD binds human CD28.

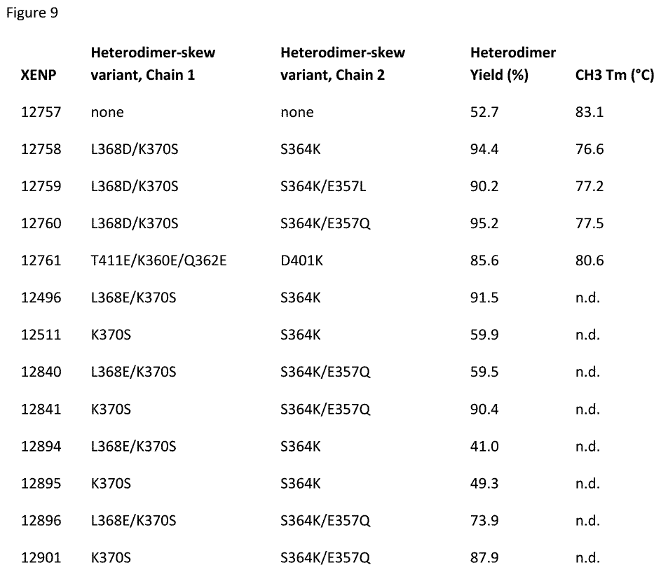

In some embodiments, the first and second Fc domains of the 2+1 mAb-scFv format antibody are variant Fc domains that include heterodimerization skew variants (e.g., a set of amino acid substitutions as shown in FIGS. 3 and 9). Particularly useful heterodimerization skew variants include S364K/E357Q: L368D/K370S; L368D/K370S: S364K; L368E/K370S S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 6). In some embodiments, the 2+1 mAb-scFv format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ charged scFv linker. In some embodiments, 2+1 mAb-scFv format antibody provided herein includes FcRn variants M428L/N434S, wherein numbering is according to EU numbering.

In some embodiments of the 2+1 mAb-scFv, the VH1 of the first and second monomer and the VL1 of the common light chain each form a PD-L1 binding domain. In some embodiments, the scFv of the second monomer is a PD-L1 binding domain.

In some embodiments, the PD-L1 binding domain is one of the following PD-L1 binding domains or a variant thereof: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31 and the Sequence Listing).

In some embodiments of the 1+1 Fab-scFv-Fc format, the PD-L1 binding domain has a variable heavy domain (VH) selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and a variable light domain (VL) selected from the group consisting of: SEQ ID NO: 1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof. In some embodiments, the VH and VL of the PD-L1 binding domain are selected from the group consisting of: a VH having an amino acid sequence of SEQ ID NO: 1183 and a VL having an amino acid sequence of SEQ ID NO: 1187; a VH having an amino acid of SEQ ID NO:1042 and a VL having an amino acid sequence of SEQ ID NO:1047; a VH having an amino acid sequence of SEQ ID NO:1159 and a VL having an amino acid sequence of SEQ ID NO: 1163; a VH having an amino acid sequence of SEQ ID NO: 1167 and a VL having an amino acid sequence of SEQ ID NO:1171; a VH having an amino acid sequence of SEQ ID NO: 1175 and a VL having an amino acid sequence of SEQ ID NO: 1179; a VH having an amino acid sequence of SEQ ID NO: 1191 and a VL having an amino acid sequence of SEQ ID NO: 1195; a VH having an amino acid sequence of SEQ ID NO: 1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

In some embodiments, the PD-L1 binding domain is selected from one of the following PD-L1 binding domains: 2.14H9 OPT, atezolizumab, avelumab, 12A4, 3G10, 10A5, h3D10 Var1, h3D10 Var2, h3D10 Var3, h3D10 Var4, h3D10 Var5, h3D10 Var6, h3D10 Var7, h3D10 Var8, h3D10 Var9, h3D10 Var10, h3D10 Var11, h3D10 Var12, h3D10 Var13, h3D10 Var14, Antibody A, C5H9v2, Humanized 29E.2A3, 1B9, 4H1, mAb-42, BAP058-03, BAP058-04, BAP058-06, BAP058-07, BAP058-11, BAP058-13, H6, RC5, SH1A1Q, SH1B3, SH1D1, SH1D2, SH1D12, SH1E1, SH1G9, SH1E6, SH1A2, SH1B1, H6B1L, H6A1, H6B1, H6B2, G12, RSA1, RA3, SH1E2, SH1E4, SH1B11, SH1C8, H1H9364P2, H1H9373P2, H1H8314N, PDL1.3.1 or a variant thereof.

In some embodiments, the VH and VL of the PD-L1 binding domain is selected from the following VH and VLs, respectively: SEQ ID NOs:89 and 93, SEQ ID NOs:97 and 101, SEQ ID NOs:105 and 109, SEQ ID NOs:113 and 117, SEQ ID NOs:121 and 125, SEQ ID NOs:129 and 133, SEQ ID NOs:137 and 141, SEQ ID NOs:145 and 149, SEQ ID NOs:153 and 157, SEQ ID NOs:161 and 165, SEQ ID NOs:169 and 173, SEQ ID NOs:177 and 181, SEQ ID NOs:185 and 189, SEQ ID NOs:193 and 197, SEQ ID NOs:201 and 205, SEQ ID NOs:209 and 213, SEQ ID NOs:217 and 221, SEQ ID NOs:225 and 229, SEQ ID NOs:233 and 237, SEQ ID NOs:241 and 245, SEQ ID NOs:249 and 253, SEQ ID NOs:257 and 261, SEQ ID NOs:265 and 269, SEQ ID NOs:273 and 277, SEQ ID NOs:281 and 285, SEQ ID NOs:289 and 293, SEQ ID NOs:297 and 301, SEQ ID NOs:305 and 309, SEQ ID NOs:313 and 317, SEQ ID NOs:321 and 325, SEQ ID NOs:329 and 333, SEQ ID NOs:337 and 341, SEQ ID NOs:345 and 349, SEQ ID NOs:353 and 357, SEQ ID NOs:361 and 365, SEQ ID NOs:369 and 373, SEQ ID NOs:374 and 381, SEQ ID NOs:385 and 389, SEQ ID NOs:393 and 397, SEQ ID NOs:401 and 405, SEQ ID NOs:409 and 413, SEQ ID NOs:417 and 421, SEQ ID NOs:425 and 429, SEQ ID NOs:433 and 437, SEQ ID NOs:441 and 445, SEQ ID NOs:449 and 453, SEQ ID NOs:457 and 461, SEQ ID NOs:465 and 469, SEQ ID NOs:473 and 477, SEQ ID NOs:481 and 485, SEQ ID NOs:489 and 493, SEQ ID NOs:497 and 501, SEQ ID NOs:504 and 508, SEQ ID NOs:512 and 516, SEQ ID NOs:520 and 524, SEQ ID NOs:528 and 534, SEQ ID NOs:537 and 541, SEQ ID NOs:545 and 549, SEQ ID NOs:553 and 557 or variants thereof.

In some embodiments, the 2+1 mAb-scFv format antibody is a bispecific antibody that binds PD-L1 and CD28. In some embodiments, the scFv of the second monomer is a CD28 binding domain and the VH1 of the first and second monomer and the VL1 of the common light chain each form PD-L1 binding domains. In some embodiments, the scFv of the second monomer is a PD-L1 binding domain and the VH1 of the first and second monomer and the VL1 of the common light chain each form CD28 binding domains.

Any suitable CD28 binding domain can be included in subject 2+1 mAb-scFv format antibody, including any of the CD28 binding domains provided herein. In some embodiments, the CD28 binding domain is one of the following CD28 binding domains or a variant thereof: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1 (FIGS. 18-23 and the Sequence Listing).

In some embodiments of the 2+1 mAb-scFv format antibody, the CD28 binding domain has a variable heavy domain (VH) selected from the group consisting of: SEQ ID NO:998, SEQ ID NO:984, SEQ ID NO:994 and SEQ ID NOs:561-629 or a variant thereof, and a variable light domain (VL) selected from the group consisting of: SEQ ID NO:1002, SEQ ID NO:984, and SEQ ID NOs:630-735 or a variant thereof. In some embodiments, the VH and VL of the CD28 binding domain are selected from the group consisting of: a VH having an amino acid sequence of SEQ ID NO:1030 and a VL having an amino acid sequence of SEQ ID NO:1034; a VH having an amino acid sequence of SEQ ID NO:1006 and a VL having an amino acid sequence of SEQ ID NO:1010; a VH having an amino acid sequence of SEQ ID NO:1014 and a VL having an amino acid sequence of SEQ ID NO:1018; and a VH having an amino acid sequence of SEQ ID NO:1022 and a VL having an amino acid sequence of SEQ ID NO:1026.

In exemplary embodiments, the CD28 binding domain includes a 1A7[CD28]_H1.14 variable heavy domain. In some embodiments, the CD28 binding domain includes a 1A7[CD28]_H1.14 variable heavy domain or variant thereof and a light variable domain of any of the CD28 binding domains provided herein. In exemplary embodiments, the CD28 binding domain is 1A7[CD28]_H1.14L1.71 or a variant thereof.

In some embodiments, the CD28 binding domain is selected from one of the following CD28 binding domains CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1 or a variant thereof.

In some embodiments, the VH and VL of the CD28 binding domain is selected from the following VH and VLs, respectively: SEQ ID NOs:1 and 5, SEQ ID NOs:9 and 13, SEQ ID NOs:17 and 21, SEQ ID NOs:25 and 29, SEQ ID NOs:33 and 37, SEQ ID NOs:41 and 46, SEQ ID NOs:49 and 53, SEQ ID NOs:57 and 61, SEQ ID NOs:65 and 69, SEQ ID NOs:73 and 77, and SEQ ID NOs:81 and 85. or variants thereof.

Any suitable PD-L1 binding domain can be included in the subject anti-PD-L1×anti-CD28 2+1 mAb-scFv format antibody, including any of the PD-L1 antigen binding domains provided herein (see, e.g., FIGS. 28-31 and the Sequence Listing). In exemplary embodiments, the PD-L1 binding domain has a VH with an amino acid sequence of SEQ ID NO:1183 and a VL with an amino acid sequence of SEQ ID NO:1187. In exemplary embodiments, the PD-L1 binding domain has a VH with an amino acid sequence of SEQ ID NO:1042 and a VL with an amino acid sequence of SEQ ID NO:1046. In exemplary embodiments, the PD-L1 binding domain has a VH with an amino acid sequence of SEQ ID NO:1159 and a VL with an amino acid sequence of SEQ ID NO:1163.

FIGS. 10-11 show some exemplary Fe domain sequences that are useful with the 2+1 mAb-scFv format. The “monomer 1” sequences depicted in FIG. 10 typically refer to the Fc domain of the “Fab-Fc heavy chain” and the “monomer 2” sequences refer to the Fc domain of the “Fab-Fc-scFv” heavy chain.” In addition, FIGS. 12-14 provides exemplary CH1 (optionally including hinge or half-hinge domains) that can be used in either the “Fab-Fc heavy chain” monomer or the “Fab-Fc-scFv” heavy chain.” FIG. 15 provides exemplary hinge domains that may be used in either the “Fab-Fc heavy chain” monomer or the “Fab-Fc-scFv” heavy chain.” Further, FIG. 16 provides useful CL sequences that can be used with this format.

An exemplary anti-PD-L1×anti-CD28×antibody in the 2+1 mAb-scFv format is depicted in FIG. 37.

6. Monospecific, Monoclonal Antibodies

As will be appreciated by those in the art, the novel Fv sequences outlined herein can also be used in both monospecific antibodies (e.g., “traditional monoclonal antibodies”) or non-heterodimeric bispecific formats. Accordingly, the present invention provides monoclonal (monospecific) antibodies comprising the 6 CDRs and/or the vh and vl sequences from the figures, generally with IgG1, IgG2, IgG3 or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody.

In some embodiments, the monospecific antibody is an anti-CD28 monospecific antibody. In certain embodiments, the monospecific anti-CD28 antibody includes the 6 CDRs of any of the following CD28 antigen binding domains or a variant thereof: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1 (FIGS. 18-23 and the Sequence Listing). In some embodiments, the monospecific anti-CD28 antibody includes the variable heavy domain and variable light domain of any of the CD28 antigen binding domains or a variant thereof: 1A7[CD28]_H1L1, 1A7[CD28]_H1.14L1, 1A7[CD28]_L1H1.14, 1A7[CD28]_H1.1L1, 1A7[CD28]_H1L1.71, 1A7[CD28]_H1.14L1.71, 1A7[CD28]_L1.71H1.14, 1A7[CD28]_H1.1L1.71, and HuTN228[CD28]_H1L1 (FIGS. 18-23 and the Sequence Listing). In some embodiments of the monospecific antibody, the CD28 binding domain has a variable heavy domain (VH) selected from the group consisting of: SEQ ID NO:998, SEQ ID NO:984, SEQ ID NO:994 and SEQ ID NOs:561-629 or a variant thereof, and a variable light domain (VL) selected from the group consisting of: SEQ ID NO:1002, SEQ ID NO:984, and SEQ ID NOs:630-735 or a variant thereof. In some embodiments, the VH and VL of the CD28 binding domain are selected from the group consisting of: a VH having an amino acid sequence of SEQ ID NO:1030 and a VL having an amino acid sequence of SEQ ID NO:1034; a VH having an amino acid sequence of SEQ ID NO:1006 and a VL having an amino acid sequence of SEQ ID NO:1010; a VH having an amino acid sequence of SEQ ID NO:1014 and a VL having an amino acid sequence of SEQ ID NO:1018; and a VH having an amino acid sequence of SEQ ID NO:1022 and a VL having an amino acid sequence of SEQ ID NO:1026.

In some embodiments, the monospecific antibody is an anti-PD-L1 monospecific antibody. In certain embodiments, the monospecific anti-PD-L1 antibody includes the 6 CDRs of any one of the following PD-L1 ABDs or a variant thereof: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31 and the Sequence Listing). In some embodiments, the monospecific anti-PD-L1 antibody includes the variable heavy domain and variable light domain of any one of the following PD-L1 ABDs or a variant thereof: 2G4[PDL1]_H1L1, 2G4[PDL1]_H1.12_L1.24, 2G4[PDL1]_H1.12_L1.66, 2G4[PDL1]_H1.12_L1.68, 2G4[PDL1]_H1.12_L1.14, 2G4[PDL1]_H1.4_L1.23, 2G4[PDL1]_H1.4_L1.24, 2G4[PDL1]_H1.9_L1, 2G4[PDL1]_H1.62_L1.63, 2G4[PDL1]_H1.6_L1.3, 2G4[PDL1]_H1.61_L1.61, 2G4[PDL1]_H1.60_L1.60, 2G4[PDL1]_H1.59_L1.60, 2G4[PDL1]_H1.14_L1.26, 2G4[PDL1]_H1_L1.27, 2G4[PDL1]_H1.8_L1.26, 2G4[PDL1]_H1_L1.25, 2G4[PDL1]_H1.10_L1.23, 2G4[PDL1]_H1.1_L1.1, 2G4[PDL1]_H1_L1.29 (FIGS. 28-31 and the Sequence Listing).

In some embodiments of the monospecific anti-PD-L1 antibody, the PD-L1 binding domain(s) has a variable heavy domain (VH) selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and a variable light domain (VL) selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof.

In some embodiments, the VH and VL of the PD-L1 binding domain is selected from the group consisting of: a VH having an amino acid sequence of SEQ ID NO: 1183 and a VL having an amino acid sequence of SEQ ID NO:1187; a VH having an amino acid of SEQ ID NO:1042 and a VL having an amino acid sequence of SEQ ID NO:1047; a VH having an amino acid sequence of SEQ ID NO: 1159 and a VL having an amino acid sequence of SEQ ID NO: 1163; a VH having an amino acid sequence of SEQ ID NO: 1167 and a VL having an amino acid sequence of SEQ ID NO:1171; a VH having an amino acid sequence of SEQ ID NO: 1175 and a VL having an amino acid sequence of SEQ ID NO: 1179; a VH having an amino acid sequence of SEQ ID NO: 1191 and a VL having an amino acid sequence of SEQ ID NO: 1195; a VH having an amino acid sequence of SEQ ID NO: 1199 and a VL having an amino acid sequence of SEQ ID NO:1203; a VH having an amino acid sequence of SEQ ID NO:1207 and a VL having an amino acid sequence of SEQ ID NO:1211; a VH having an amino acid sequence of SEQ ID NO:1215 and a VL having an amino acid sequence of SEQ ID NO:1219; a VH having an amino acid sequence of SEQ ID NO:1223 and a VL having an amino acid sequence of SEQ ID NO:1227; a VH having an amino acid sequence of SEQ ID NO:1231 and a VL having an amino acid sequence of SEQ ID NO:1235; a VH having an amino acid sequence of SEQ ID NO:1239 and a VL having an amino acid sequence of SEQ ID NO:1243; a VH having an amino acid sequence of SEQ ID NO:1247 and a VL having an amino acid sequence of SEQ ID NO:1251; a VH having an amino acid sequence of SEQ ID NO:1255 and a VL having an amino acid sequence of SEQ ID NO:1259; a VH having an amino acid sequence of SEQ ID NO:1263 and a VL having an amino acid sequence of SEQ ID NO:1267; a VH having an amino acid sequence of SEQ ID NO:1271 and a VL having an amino acid sequence of SEQ ID NO:1275; a VH having an amino acid sequence of SEQ ID NO:1279 and a VL having an amino acid sequence of SEQ ID NO:1283; a VH having an amino acid sequence of SEQ ID NO:1287 and a VL having an amino acid sequence of SEQ ID NO:1291; a VH having an amino acid sequence of SEQ ID NO:1295 and a VL having an amino acid sequence of SEQ ID NO:1299; and a VH having an amino acid sequence of SEQ ID NO:1303 and a VL having an amino acid sequence of SEQ ID NO:1307.

### VI. Nucleic Acids

In another aspect, provided herein are nucleic acid compositions encoding the antigen binding domains and anti-PD-L1 and anti-CD28 antibodies provided herein (e.g., PD-L1×αCD28 bispecific antibodies).

As will be appreciated by those in the art, the nucleic acid compositions will depend on the format and scaffold of the heterodimeric protein. Thus, for example, when the format requires three amino acid sequences, such as for the 1+1 Fab-scFv-Fc, 2+1 Fab₂-scFv-Fc, and 2+1 mAb-scFv formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer: second monomer:light chains (in the case of many of the embodiments herein that have three polypeptides comprising the heterodimeric antibody) in a 1:1:2 ratio, these are not the ratios that give the best results.

The antibodies and ABDs provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VII. Biological and Biochemical Functionality of the Anti-PD-L1×Anti-CD28 Antibodies

Generally the bispecific anti-PD-L1×anti-CD28 antibodies described herein are administered to patients with cancer (e.g., a PD-L1 associated cancer or cancer that has been shown to benefit from anti-PD-1 or anti-PD-L1 treatment), and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

A. Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions.

### VIII. Treatments

Once made, the compositions of the invention find use in a number of oncology applications, by treating cancer, generally by enhancing immune responses (e.g., T cell activation and proliferation), particularly when used with anti-cancer therapies such as anti-PD1 and anti-tumor bispecific antibodies. In some embodiments, the antibodies provided herein enhance immune responses (e.g., T cell activation and proliferation) by providing agonistic costimulation of T cells in the microenvironment of tumors expressing PD-L1.

A. Anti-CD28×Anti-TAA/Anti-TAA Bispecific Antibody

In some embodiments, the anti-PD-L1×anti-CD28 bispecific antibodies provided herein are administered with an anti-tumor bispecific antibody. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 32A). In contrast, only the first signal is provided in treatment with CD3 bispecific antibodies that target a TAA (i.e., anti-CD3×anti-TAA bispecific antibodies).

Without being bound by any particular theory of operation, it is believed that the anti-PD-L1×anti-CD28 bispecific antibodies provided herein can enhance the anti-tumor response of an anti-CD3×anti-TAA bispecific antibody by providing CD28 costimulation and blockage of inhibitory PD-L1.PD-1 pathway interactions (see FIGS. 32B and 34 and Example 4B). Thus, in one aspect, provided herein are methods of methods of treating a cancer in a patient by administering the patient an anti-CD3×anti-TAA bispecific antibody and an anti-CD28×anti-PD-L1 bispecific antibody provided herein. In some embodiments, the administration of the anti-CD3×anti-TAA bispecific antibody and anti-CD28×anti-PD-L1 bispecific antibody enhances an immune response against the tumor in the patient. CD3 binding domains that can be included in the anti-CD3×anti-TAA bispecific antibodies are included in FIG. 44.

B. Administrative Modalities

The antibodies provided herein administered to a subject, in accord with known methods, such as intravenous administration as a bolus or by continuous infusion over a period of time.

C. Treatment Modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, 2014/0288275 and WO2014/145806, all of which are expressly incorporated by reference in their entirety and particularly for the techniques outlined therein.

### BACKGROUND

While checkpoint blockade immunotherapies have proven to be effective, many patients nonetheless fail to achieve a response. Engagement of T cell costimulatory receptors on TILs (e.g., CD80/CD86) with agonistic antibodies (e.g., anti-CD28 antibodies) could provide an additional positive signal capable of overcoming negative signals of immune checkpoints and may be a useful therapeutic modality to stack with checkpoint blockade.

Anti-PD-L1×anti-CD28 bispecific antibodies provided herein provide a costimulatory signal for T-cell activation against tumor cells while advantageously also block inhibitory PD-L1:PD1 pathway interactions.

### Example 1: CD28 Binding Domains

Sequences for human, mouse, and cynomolgus PD-L1 are depicted in FIG. 1 and are useful for the development of cross-reactive CD28 antigen binding domains for ease of clinical development.

### 1A: Novel CD28 Binding Domains

An approach considered to avoid the superagonism associated with TGN1412 was to generate novel CD28 binding domains having lower affinity binding to CD28 and/or binding to a different CD28 epitope than TGN1412. In one campaign to generate such novel CD28 binding domains, in-house de novo phage libraries were panned against CD28. In another campaign, rat hybridomas were used to generate additional CD28 binding domains.

1A(a): Phage-Derived Clone 1A7

It should be noted that this phage library utilized a human germline VL with diversity introduced into the LCDR3. The amino acid sequences for exemplary phage-derived clone 1A7 are depicted in FIG. 18.

The phage-derived clones were formatted as bivalent mAbs to investigate their binding characteristics. Plasmids containing the variable heavy and variable light domains of select clones were constructed by Gibson assembly and subcloned into a pTT5 expression vector containing the coding sequence for the IgG1 constant regions (with E233P/L234V/L235A/G236del/S67K ablation variants). DNA was transfected in HEK293E for expression and resulting bivalent mAbs were purified from the supernatant using protein A chromatography.

Affinity of the phage-derived bivalent mAbs for CD28 was screened using Octet, a BioLayer Interferometry (BLI)-based method. Experimental steps for Octet generally include the following: Immobilization (capture of ligand to a biosensor); Association (dipping of ligand-coated biosensors into wells containing the analyte); and Dissociation (returning of biosensors to well containing buffer). The resulting apparent dissociation constant (K_{Dapp}) are depicted in FIG. 24 for XENP28428 (based on clone 1A7) and additional phage-derived comparators.

Binding of the phage-derived bivalent mAbs to cell-surface CD28 was investigated. Human PBMCs were incubated with indicated concentrations of XENP28428 or comparator phage-derived mAbs for 1 hour at 4° C. Cells were then then stained with Alexa Fluor® 647 AffiniPure F(ab′)₂ Fragment Goat Anti-Human IgG, Fcγ fragment specific secondary antibody (Jackson ImmunoResearch, West Grove, Pa.) for 1 hour at 4° C. and analyzed by flow cytometry. The data (FIG. 25) show that the phage-derived mAbs were able to bind human PBMCs, although with much weaker maximum binding than prior art anti-CD28 mAb HuTN228 (XENP27181, sequences for which are depicted in FIG. 23).

1A(b): 1A7 is not Superagonistic

Potential superagonism of XENP34339 was assessed by air-drying per the Stebbings protocol (Stebbings R. et al. 2007). Air-drying of test articles was achieved by drying in a SpeedVac™ for 2 hours at room temperature. Human PBMCs were treated for 24 hours with 10 μg of air-dried XENP28428 (parental αCD28 mAb 1A7), and activity was compared to the superagonist TGN1412 (XENP29154; sequences for which are depicted in FIG. 45) or PBS control. Air-dried TGN1412 promoted IFNγ secretion from unstimulated human PBMC. In comparison, IFNγ level in PBMCs treated with air-dried XENP28428 remained similar to the negative control of PBS (data shown in FIG. 46).

1A(c): Engineering 1A7 Affinity Variants

Towards optimization of αPDL1×αCD28 bsAbs as described in Example 5, numerous 1A7 affinity variants were developed by engineering VH variants (illustrative sequences as depicted in FIG. 19 and additional sequences depicted as SEQ ID NOs:994-1001), VL variants (illustrative sequences as depicted in FIG. 20 and additional sequences depicted as SEQ ID NOs:1002-1005), and combinations thereof (illustrative sequences for which are depicted in FIG. 21). Affinity of illustrative variants, in the context of αPDL1×αCD28 bsAbs having sequences as depicted in FIG. 22, was determined using Octet as generally described above, sensorgrams and binding affinity as depicted in FIG. 47.

1B: Additional CD28 Binding Domains

Sequences for additional CD28 binding domains which may find use in the αPDL1×αCD28 bsAbs of the invention are depicted as SEQ ID NOs:1006-1021.

### Example 2: PDL1 Binding Domains

Sequences for human, mouse, and cynomolgus PD-L1 are depicted in FIG. 2 and are useful for the development of cross-reactive PDL1 antigen binding domains for ease of clinical development.

2A: Novel PDL1 Binding Domains

Numerous novel PDL1 binding domains were generated by hybridoma technology through IMMUNOPRECISE®. In addition to targeting the αPDL1×αCD28 to PDL1+ cells, it was contemplated that additional blockade of PDL1:PD1 interaction would be useful. Further, for ease of clinical development, it is useful to investigate various parameters of the bispecific antibodies of the invention such as pharmacodynamics, pharmacokinetics, and toxicity in cynomolgus monkeys.

Accordingly, the novel hydridoma-derived PDL1 binding domains were screened for affinity to human and cynomolgus PDL1 as well as for their ability to block PDL1:PD1 interaction using Octet as generally described above. In particular, anti-mouse IgG biosensor was dipped into 1:3 dilution of hybridoma culture supernatant and then dipped into multiple concentrations of human or cynomolgus PDL1 antigen (to determine binding affinity) or into mixtures of PD1:PDL1 antigen (blocking antibodies will not bind the PD1:PDL1 complex). Out of 106 hybridoma-derived PDL1 binding domains investigated, only 70 were cross-reactive for human and cynomolgus PDL1. Out of those 70, only 33 were considered as complete blockers of PD1:PDL1 interaction. The remaining PDL1 binders were further characterized for ability to bind PDL1 on cells and block PDL1:PD1 interaction on cells and affinity. Finally, suitable PDL1 binding domains were humanized using string content optimization (see, e.g., U.S. Pat. No. 7,657,380, issued Feb. 2, 2010). The amino acid sequences for exemplary humanized hybridoma-derived clone 2G4 (selected based on the above filtering) are depicted in FIG. 28.

2a(a): Comparison to Other PDL1 Binding Domains

As described above, numerous other PDL1 binding domains were identified. In a first experiment comparing anti-PDL1 clone 2G4 with the other PDL1 binding domains. anti-PDL1 mAbs were incubated with indicated concentration of huPDL1-mFc fusions. The mixture was then combined with PD-1-expressing HEK293T cells. Cells were stained with anti-mouse Fc and binding was assessed by flow cytometry, data for which are depicted in FIG. 41. The data show that anti-PDL1 clone 2G4 blocks PD1:PDL1 interaction.

In a second experiment, CD3+ T cells from 11 unique donors were treated with a constant dose of the indicated test articles in the presence of 10,000 dendritic cells. Cytokine secretion was measured using MSD assay (Meso Scale Discovery, Rockville, Md.). The data depicted in FIG. 42 show that anti-PDL1 2G4 induced greater IL-2 and IFNγ release in comparison to the partial-blocking and the non-blocking anti-PDL1 clones.

2A(b): Engineering 2G4 Affinity Variants

Towards optimization of αPDL1×αCD28 bsAbs as described in Example 5, numerous 2G4 affinity variants were developed by engineering VH variants (illustrative sequences as depicted in FIG. 29 and additional sequences depicted as SEQ ID NOs:1055-1102), VL variants (illustrative sequences as depicted in FIG. 30 and additional sequences depicted as SEQ ID NOs:1103-1158), and combinations thereof (illustrative sequences as depicted in FIG. 31). Affinity of illustrative variants, in the context of αPDL1×αCD28 bsAbs having sequences as depicted in FIG. 28, was determined using Biacore. Experimental steps for Biacore generally included the following: Immobilization (capture of ligand onto a sensor chip); Association (flowing of various concentrations of analyte over sensor chip); and Dissociation (flowing buffer over the sensor chips) in order to determine the affinity of the test articles. A reference flow with buffer alone was also included in the method for background correction during data processing. Binding affinities and kinetic rate constants were obtained by analyzing the processed data using a 1:1 binding model. Binding affinity for human and cynomolgus PDL1 determined as such are depicted in FIG. 50.

2B: Additional PDL1 Binding Domains

Sequences for additional PDL1 binding domains which may find use in the αPDL1×αCD28 bsAbs of the invention are depicted as SEQ ID NOs:1159-1310.

### Example 3: Engineering αPDL1×αCD28 bsAbs

As described in Example 4, in classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 32). Further, it may be useful to stack the CD28 signal with checkpoint blockade to mitigate any checkpoint mediated repression of the added CD28 signal (FIG. 33). αPDL1×αCD28 may provide the Signal 2 while advantageously further enabling blockade of PDL1:PD1 interaction (FIG. 34).

Accordingly, αPDL1×αCD28 bsAbs were engineered and produced. A number of formats were conceived, illustrative formats for which are outlined below and in FIGS. 26-27. It should be noted that in each case, the CD28 bispecific antibodies are monovalent for CD28 and incorporate Fc variants engineered to ablate FcγR binding (such as those depicted in FIG. 5) to avoid potential superagonism.

3A: 1+1 Fab-scFv-Fc Format

One format utilizing Fab domains and scFv is the 1+1 Fab-scFv-Fc format (depicted schematically in FIG. 27A) which comprises a first monomer comprising a single-chain Fv (“scFv”) with a first antigen binding specificity covalently attached to a first heterodimeric Fc domain, a second monomer comprising a heavy chain variable region (VH) covalently attached to a complementary second heterodimeric Fc domain, and a light chain (LC) transfected separately so that a Fab domain having a second antigen binding specificity is formed with the variable heavy domain. Sequences for illustrative αPDL1×αCD28 bsAbs (based on binding domains as described in Examples 1 and 2) in the 1+1 Fab-scFv-Fc format are depicted in FIG. 35.

3B: 2+1 Fab2-scFv-Fc Format

Another such format is the 2+1 Fab2-scFv-Fc format (depicted schematically in FIG. 27B) which comprises a first monomer comprising a VH domain covalently attached to an scFv (having a first antigen binding specificity) covalently attached to a first heterodimeric Fc domain, a second monomer comprising a VH domain covalently attached to a complementary second heterodimeric Fc domain, and a LC transfected separately so that Fab domains having a second antigen binding specificity are formed with the VH domains. Sequences for illustrative αPDL1×αCD28 bsAbs (based on binding domains as described in Examples 1 and 2) in the 2+1 Fab2-scFv-Fc format are depicted in FIG. 36.

3C: 2+1 mAb-scFv Format

An additional format utilizing Fab domains and scFv is the 2+1 mAb-scFv format (depicted schematically in FIG. 27E) which comprises a first monomer comprising a VH domain covalently attached to a first heterodimeric Fc domain covalently attached to an scFv (having a first antigen binding specificity), a second monomer comprising a VH domain covalently attached to a complementary second heterodimeric Fc domain, and a LC transfected separately so that Fab domains having a second antigen specificity are formed with the VH domains. Sequences for illustrative αPDL1×αCD28 bsAbs (based on binding domains as described here) in the 2+1 mAb-scFv format are depicted in FIG. 37.

### Example 4: Characterizing Prototype αPDL1×αCD28 bsAbs

4A: αPDL1×αCD28 bsAbs Induce Expansion of T Cells

MDA-MB-231 (PDL1+) cancer cells were ectopically loaded with pp65-derived NLV-peptide for 24 hours. The following day, T cells from a CMV+ donor were added along with a monovalent αPDL1 antibody XENP24118 (based on avelumab; sequences depicted in FIG. 38) or αPDL1×αCD28 XENP34963. Expansion of CMV+ T cells was measured with an NLV-specific tetramer. The data as depicted in FIG. 39 show that the αPDL1×αCD28 bsAb XENP36233 significantly enhanced T cell expansion in comparison to PDL1 blockade alone.

4B: αPDL1×αCD28 bsAbs Block PD-1:PD-L1 Interaction During T-Cell:Cancer Cell Interactions

Jurkat-PD1 cells were treated with a dose titration of the indicated test articles (i.e. αPDL1×αCD28 bsAbs containing αPDL1 clone 2G4, a partial-blocking αPDL1 arm, or a non-blocking αPDL1 arm) in the presence of CHO-PDL1-CD80-αCD3 and CHO-PDL1-αCD3 cells. 6 hours post Jurkat-PD1 seeding, luciferase activity was measured as an indicator of PD-1 blockade. The data as depicted in FIG. 51 show that XENP36233 having blocking αPDL1 clone 2G4 induced strong luciferase activity in comparison to αPDL1×αCD28 bsAbs having partial blocking or non-blocking αPDL1 arms.

4C: αPDL1×αCD28 bsAbs are More Active on Cells Having Higher PDL1 Antigen Density

PDL1^{null} MC38 cells were transfected to express low or medium levels of PDL1 antigen (respectively 40K and 59K antigen density). Parental, PDL1^{low}, and PDL1^{med} MC38 clones were treated with a dose titration of XENP36233. Secondary A647-conjugated anti-human Fc antibody was used to detect the bsAbs by flow cytometry, data for which are depicted in FIG. 52. The data show that binding correlates to PDL1 antigen density.

Next, PDL1^{null} HEK293T cells were transfected to express medium or high levels of PDL1 antigen. 10,000 CD3+ T cells were treated with a dose titration of XENP36233 and 1 μg/mL αB7H3×αCD3 in the presence of 10,000 PDL1-HEK293T density series. 1 day post T cell seeding, IL-2 secretion was measured using MSD assay. The data as depicted in FIG. 53 show that αPDL1×αCD28 bsAbs are more active on cells having higher PDL1 antigen density.

4D: αPDL1×αCD28 bsAbs Enhance Activity of CD3 Bispecific Antibodies

In a first experiment, induction of cytokine (IL-2 and IFNγ) release and CD3+ T cell expansion by αPDL1×αCD28 bsAbs in combination with CD3 bispecific antibodies was assessed using Incucyte® Live-Cell Analysis system (Essen BioScience, Ann Arbor, Mich.). MDA-MB-231 cancer cells and T cells were mixed with 1 μg/ml of an illustrative αB7H3×αCD3 bispecific and indicated concentrations of αPDL1 mAb XENP24118 or αPDL1×αCD28 bsAb XENP34963. Cancer cell growth was assayed over time with Incucyte, while cytokine secretion was measured using MSD assay (Meso Scale Discovery, Rockville, Md.) and T cell expansion was measured using flow cytometry. The data as depicted in FIG. 40 show that αPDL1×αCD28 bsAb synergizes with CD3 bispecific antibodies to induce T-cell activation.

In another experiment, cell kill at a 1:1 effector:target ratio was assessed using xCELLigence Real Time Cell Analysis instrument (ACEA Biosciences, San Diego, Calif.). 1,250 LNCaP cancer cells were first seeded. After 48 hours, freshly enriched CD3+ T cells at an effector:target of 1:1 were added along with antibodies (αPSMA×αCD3 XENP32220 alone or XENP32220 in combination with XENP36233; sequences for XENP32220 are depicted in FIG. 41) at a constant dose. Cell kill was recorded for 10 days post T cell seeding. In this experiment, the data as depicted in FIG. 42 show that XENP32220 alone was able to enhance cell kill in comparison to incubation of cancer and T cells alone; however, addition of PDL1×CD28 overcomes cancer cell resistance to the CD3 bispecific and further enhances cell kill.

4E: αPDL1×αCD28 bsAbs do not Synergize with CD3 bsAbs on PDL1 Negative Cell Lines

CD3+ T cells were incubated with 22Rv1 (PDL1^{null}) at 10:1 and 1:1 E:T ratios with αB7H3×αCD3 alone or αB7H3×αCD3 in combination with XENP36233. Cell kill was recorded using xCELLigence for 7 days post T cell seeding. The data as depicted in FIG. 54 show that αPDL1×αCD28 bsAb XENP36233 did not synergize with the αB7H3×αCD3 to enhance cell kill of the PDL1 negative 22Rv1 cell line.

4F: αPDL1×αCD28 Enhances Anti-Tumor Activity In Vivo

MC38 cancer cells stably expressing human PDL1 antigen were subcutaneously inoculated into human CD28 knock-in mice. When tumors were palpable (Day 0), mice were intraperitoneally treated weekly with either 5 mg/kg bivalent αPDL1 antibody based on avelumab (XENP24118), 6 mg/kg αPDL1×αCD28 XENP34961 (2+1 mAb-scFv format), or 8.3 mg/kg αPDL1×αCD28 XENP34963 (1+1 Fab-scFv-Fc format). Mice were additionally dosed on Days 3, 7, 10, 14, and 17. Tumor volumes were monitored by caliper measurements, data for which are shown (days post 1^{st} dose) in FIG. 43. The data show that although PDL1 blockade inhibited tumor growth, the αPDL1×αCD28 bsAbs as a single-agents significantly enhanced anti-tumor activity.

4G: αPDL1×αCD28 was Well Tolerated in Cynomolgus Monkeys and Exhibited Favorable Pharmacokinetics

Cynomolgus monkeys were dosed with XENP36764 (sequences for which are depicted in FIG. 35; XENP36233 further engineered with M428L/N434S for enhanced half-life). The data as depicted in FIG. 55 show that the αPDL1×αCD28 exhibited favorable pharmacokinetics. Additionally, the bsAb was well tolerated (data not shown).

### Example 5: Optimizing αPDL1×αCD28 bsAbs

5A: Mechanism-Based PK/PD Modeling Suggests Avenues for Tuning CD28 and PDL1 Binding Affinities

Surface expression of PD-L1 is low on tumors may limit synapse engagement and presents the problem of how to obtain optimal cross-linking of CD28 with a bispecific. With this background, computer modeling was used to assist engineering of optimized αPDL1×αCD28 bsAbs by finding the best balance of CD28 and PDL1 affinities to achieve low antigen density targeting. A mechanism-based model was developed using experimental data (from scientific literature and in-house generated) and the following assumptions (schematically depicted in FIG. 56): three well-mixed compartments (central, peripheral, and tumor); all the compartments contain both T cells and PDL1 expressing cells; free drug transport between all compartments; non-specific clearance of the drug occurs in all compartments with the same order rate; soluble target for CD28 and PDL1 are low affinity and therefore not included; drug-bound receptor has the same internalization rate as the free receptor; cross-linking CD28:drug_PD-L1 trimer is assumed to internalize with either target; and cross-linking CD28:drug_PD-L1 trimer is assumed to internalize at the rate of faster receptor. As depicted in FIG. 57, the model predicted intratumoral T cell costimulatory activity and consistent PDL1 blockade; linear PK at dose levels consistent with typical checkpoint inhibitor regimens; trimer formation in the tumor indicating costimulation; and consistent blockade of PDL1.

Notably in view of low surface expression of PDL1 on tumors, the simulations suggested that CD28 affinity must be enhanced (from 560 nM to 58 nM) and that PDL1 affinity must be enhanced (from 2.4 nM to 0.1 nM).

5B: Tuning CD28 Binding Affinity

Based on the above suggestion, αCD28 clone 1A7 affinity variants were engineered as described in Example 1A(c) and paired with WT αPDL1 clone 2G4.

5B(a): Increasing CD28 Affinity Leads to More Potent and Efficacious TL-2 Secretion

In a first experiment investigating the effect of enhanced CD28 binding affinity, MDA-MB-231 or DU145 cancer were incubated with CD3+ T cells at an E:T ratio of 10:1, 1 μg/mL αB7H3×αCD3, and titrated doses of the indicated αPDL1×αCD28 test articles having the following range of CD28 binding affinities: 37 nM (1A7_L1.71_H1.14 in XENP37561); 36 nM (1A7_H1.14_L1.71 in XENP37261); 96 nM (1A7_H1.1_L1.71 in XENP37560); 180 nM (1A7_H1_L1.71 in XENP37559); 286 nM (1A7_L1_H1.14 in XENP37412); and 240 nM (1A7_H1.14_L1 in XENP36233). IL-2 secretion was assessed after 24 hours, data for which are depicted in FIG. 58. The data show that increasing CD28 affinity leads to more potent and efficacious IL-2 secretion. Notably in this set, 180 nM binding affinity for CD28 is the minimum for maximum activity (with XENP37412 and XENP36233 respectively having 286 nM and 240 nM binding affinities for CD28 demonstrating not only less potent but also less efficacious activity).

5B(b): Increasing CD28 Affinity Increases Cytotoxicity of PDL1^{null} Cancer Cells at an E:T of 1:1

In a second experiment investigating the effect of enhanced CD28 binding affinity, LnCAP cells (PDL1^{low}) were incubated with CD3+ T cells at a low E:T ratio of 1:1 with 1 μg/ml αB7H3×αCD3 bsAb alone or in combination with 1 μg/ml of αPDL1×αCD28 bsAbs having different CD28 binding affinities (same set as in Example 5B(a)). Cytotoxicity was recorded using xCELLigence for 6 days post T cell seeding, data for which are depicted in FIG. 59. The data show that increasing CD28 affinity increases cytotoxicity of low PDL1 expressing cancer cells at a low E:T ratio of 1:1.

5B(c): Increasing CD28 Affinity Increases Cytotoxicity of PDL1^{med} Cancer Cells at an E:T of 0.1:1

In a second experiment investigating the effect of enhanced CD28 binding affinity, DU145 cells (PDL1^{med}) were incubated with CD3+ T cells at a low E:T ratio of 0.1:1 with 1 μg/ml a1B7H3×αCD3 bsAb alone or in combination with 1 μg/ml of αPDL1×αCD28 bsAbs having different CD28 binding affinities (same set as in Example 5B(a)). Cytotoxicity was recorded using xCELLigence for 6 days post T cell seeding, data for which are depicted in FIG. 60. The data show that increasing CD28 affinity increases cytotoxicity of medium PDL1 expressing cancer cells at a very low E:T ratio of 0.1:1.

5B(d): αPDL1×αCD28 bsAbs Tuned for Enhanced CD28 Binding Affinity are Active In Vivo

αPDL1×αCD28 bsAb XENP37261 having affinity-enhanced CD28 arm was investigated in an in vivo anti-tumor model similar to Example 4F. MC38 cancer cells stably expressing human PDL1 antigen were subcutaneously inoculated into human CD28 knock-in mice. When tumors were palpable (Day 0), mice were intraperitoneally treated weekly with either 6.6 mg/kg monovalent αPDL1 antibody based on avelumab (XENP36627) or 8.3 mg/kg αPDL1×αCD28 XENP37261. Tumor volumes were monitored by caliper measurements, data for which are shown (days post 1^{st} dose) in FIG. 61. The data show that although PDL1 blockade inhibited tumor growth, the αPDL1×αCD28 bsAb XENP37261 as a single-agent significantly enhanced anti-tumor activity.

5C: Tuning PDL1 Binding Affinity

Next, αPDL1 clone 2G4 affinity variants were engineered as described in Example 1A(c) and paired with αCD28 clone 1A7_H1_L1.71 (180 nM CD28 arm; lower CD28 activity allows for assessment of PDL1 blockade).

5C(a): Increasing PDL1 Affinity Promotes Cytokine Secretion

In a first experiment, DU145-NLR cells were incubated with CD3+ T cells at a E:T ratio of 1:1 and treated with 1 μg/mL αB7H3×αCD3 and dose response of the indicated αPDL1×αCD28 bsAbs having different PDL1 binding affinities. 1 day post T cell seeding, IL-2 secretion was assessed using MSD assay, data for which are depicted in FIG. 62. The data show that increased PDL1 affinity promotes IL-2 secretion.

In a second experiment, data for which are depicted in FIG. 63, XENP38514 having 0.17 nM PDL1 arm enhances T cell/APC interaction in DC:T cell mixed lymphocyte reaction as indicated by IL-2 and IFNγ release.

5C(b): Increasing PDL1 Affinity Promotes PD1:PDL1 Blockade

In another experiment, indicated concentrations of the αPDL1×αCD28 bsAbs were incubated in the presence of huPD-L1-mFc fusion for 30 minutes at room temperature. The mixture was then combined with PD-1-expressing HEK293T cells and incubated for 1 hour at 4° C. Cells were stained with anti-mouse Fc and binding was assessed by flow cytometry, data for which are depicted in FIG. 64. The data show the αPDL1×αCD28 bsAbs can block interaction between PD1 and PDL1.

5D: Tuning Both CD28 and PDL1 Binding Affinities

As the mechanism-based modeling suggested that affinity-enhanced CD28 binding domains should be paired with affinity-enhanced PDL1 binding domains, bsAbs having affinity-enhanced αCD28 clone 1A7 variants were paired with affinity-enhanced αPDL1 clone 2G4.

In a first experiment, PDL1 binding domains having 0.6 nM, 1 nM, and 2.6 nM binding affinities were paired with CD28 binding domains having 37 nM, 96 nM, 180 nM, and 230 nM binding affinities and investigated in an SEB-stimulated PBMC assay. PBMCs were stimulated with 110 ng/mL SEB for 2 days. 200,000 PBMCs were then seeded in the presence of 10 ng/mL SEB and αPDL1×αCD28 bsAbs at the indicated doses. TL-2 secretion was assayed after 24 hours, data for which are depicted in FIG. 65. The data suggest that high affinity PD-L1 arms such as 0.6 nM arm is optimally paired with 37 nM CD28 arm; nonetheless, high affinity PD-L1 binding overcomes the requirement for increased CD28 affinity as it also enables good activity in combination with low affinity CD28 as in XENP39365 and XENP39368.

### Example 6: Further Optimization of the αPDL1×αCD28 bsAbs

Additional optimization of other characteristics such as stability and expression are also important from a developability perspective. Although pairing high affinity PDL1 binding domain 2G4_H1.12_L1.24 with high affinity CD28 binding domain 1A7_H1.14_L1.71 as in XENP38512 enables a molecule having optimal biological activity, it turns out that XENP38512 suffered from less than ideal stability and antibody expression levels (data not shown). Accordingly, additional affinity variants were generated based on 2G4_H1.12_L1.24 by reverting or back mutating substitutions suspected to decrease stability and/or yield and/or to achieve a minimal necessary set of substitutions (relative to H1L1). Several binding domains including 2G4_H1.12_L1.14, 2G4_H1.12_L1.66, and 2G4_H1.12_L1.68 were identified which demonstrated 4.5° C. improved Tm in comparison to 2G4_H1.12_L1.24 as well as improved yield while maintaining high affinity PDL1 binding. Additionally, codon optimization was investigated.

In a further experiment, PDL1 binding domains 2G4_H1.12_L1.24 (0.17 nM), 2G4_H1.12_L1.66 (0.86 nM), 2G4_H1.12_L1.68 (0.80 nM), or 2G4_H1.12_L1.14 (0.42 nM) binding affinities were paired with CD28 binding domain having 37 nM binding affinity (1A7_H1.14_L1.71) and investigated in a reverse CAR-T experiment (wherein test articles were incubated with CD3+ enriched T cells, MDA-MB-231 transfected to express αCD3 scFv (to act as Signal 1)), and 1 μg/mL of an illustrative B7H3×CD3 bsAb. It should be noted that XENP38512 and XENP40036 have the same amino acid but were expressed from different codons. The data as depicted in FIG. 66 show that the stability/production optimized variants enabled similar IL2 induction as compared to 2G4_H1.12_L1.24 albeit with XENP40409 (non-Xtend analog of XENP40706, containing 2G4_H1.12_L1.14) inducing IL2 production most potently.

### Example 7: PDL1×CD28 bsAbs Selectively Induce Proliferation of Effector T Cells in Cynomolgus Monkeys

Cynomolgus monkeys were dosed with XENP36803 (1× dose, 4× dose, and 10× dose) or XENP36764 (4× dose, 10× dose, and 20× dose) to investigate pharmacodynamics of the PDL1×CD28 bsAbs of the invention. Data from the study show that CD28 receptor occupancy is detected up to day 14 on T cells (FIG. 67) and PDL1 receptor occupancy is detected in activated monocytes (FIG. 68). Finally as depicted in FIG. 70, it was found that the PDL1×CD28 bsAbs demonstrate cytokine-like activity in selectively inducing proliferation of effector CD4+ and CD8+ T cells, particularly CD45RA-subsets. Notably, XENP36764 in the 1+1 Fab-scFv-Fc format having monovalent binding for PDL1 induced proliferation more efficaciously than XENP36803 in the 2+1 mAb-scFv format having bivalent binding for PDL1.

What is claimed is:

1. A heterodimeric antibody comprising:
a) a first monomer comprising:
i) a scFv comprising a first variable heavy domain, an scFv linker and a first variable light domain; and
ii) a first Fe domain, wherein the scFv is covalently attached to the N-terminal of the first Fc domain using a domain linker;
b) a second monomer comprising, from N-terminus to C-terminus, a VH1-CH1-hinge-CH2-CH3, wherein VH is a first variable heavy domain and CH2-CH3 is a second Fc domain; and
c) a light chain comprising, from N-terminus to C-terminus, VL1-CL, wherein VL1 is a variable light domain and CL is a constant light domain,
wherein said VH1 and said VL1 form a first antigen binding domain (ABD) and wherein said scFv comprises a second VH domain (VH2), a scFv linker, and a second VL domain (VL2), wherein said VH2 and said VL2 form a second ABD, and wherein one of said ABDs binds CD28 and the other binds PD-L1.

2.-16. (canceled)

17. A heterodimeric antibody comprising:
a) a first monomer comprising from N- to C-terminus, VH1-CH1-first domain linker-scFv-second domain linker-CH2-CH3,
wherein VH1 is a first variable heavy domain, and CH2-CH3 is a first Fc domain;
b) a second monomer comprising from N- to C-terminus, a VH1-CH1-hinge-CH2-CH3, wherein CH2-CH3 is a second Fc domain; and
c) a light chain comprising, from N- to C-terminus, VL1-CL, wherein VL1 is a variable light domain and CL is a constant light domain,
wherein each of the VH1 domains and the first VL1 domain together form a first antigen binding domain (ABD) and the scFv comprises a second VH domain (VH2), a scFv linker, and a second VL domain (VL2), and the VH2 and the VL2 form a second ABD, wherein one of the first and second ABDs bind human CD28 and the other of the first and second ABDs binds PD-L1.

18.-34. (canceled)

35. A heterodimeric antibody comprising:
a) a first monomer comprising from N-terminus to C-terminus, VH1-CH1-hinge-CH2-CH3-domain linker-scFv,
wherein VH1 is a first variable heavy domain, and CH2-CH3 is a first Fc domain;
b) a second monomer comprising from N-terminus to C-terminus a VH1-CH1-hinge-CH2-CH3, wherein CH2-CH3 is a second Fc domain; and
c) a light chain comprising, from N-terminus to C-terminus, VL1-CL, wherein VL1 is a variable light domain and CL is a constant light domain,
wherein each of the VH1 domain and the first VL1 domain together form a first antigen binding domain (ABD) and the scFv comprises a second VH domain (VH2), a scFv linker, and a second VL domain (VL2), and the VH2 and the VL2 together form a second ABD, wherein one of the first and second ABDs bind human CD28 and the other of the first and second ABDs bind PD-L1.

36.-57. (canceled)

58. A composition comprising an anti-PD-L1 Antigen Binding Domain (ABD) comprising:
a) a variable heavy domain (VH) with an amino acid sequence selected from the group consisting of: SEQ ID NO:1079, SEQ ID NO:1042, SEQ ID NO:1055, SEQ ID NO:1059, SEQ ID NO:1063, SEQ ID NO: 1067, SEQ ID NO: 1071, SEQ ID NO: 1075, SEQ ID NO:1083, SEQ ID NO:1087, SEQ ID NO:1091, SEQ ID NO:1095, SEQ ID NO:1099, and SEQ ID NOs:736-797 or a variant thereof, and
b) variable light domain (VL) with an amino acid sequence selected from the group consisting of: SEQ ID NO:1111, SEQ ID NO:1046, SEQ ID NO:1103, SEQ ID NO:1107, SEQ ID NO:1115, SEQ ID NO:1119, SEQ ID NO:1123, SEQ ID NO:1127, SEQ ID NO:1131, SEQ ID NO:1135, SEQ ID NO:1139, SEQ ID NO:1143, SEQ ID NO:1147, SEQ ID NO:1151, SEQ ID NO:1155, and SEQ ID NOs:798-868 or a variant thereof.

59.-67. (canceled)
